SEQUENCE LISTING (78 sequences)

SEQ ID NO: 1 - protein, 640 aa, Artificial
  Artificial construct
NYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLD

SEQ ID NO: 2 - protein, 341 aa, Plasmodium falciparum
KCDKCKSGTSRSRKIWTWRKSSGNKEGLQEEYANTIGLSPRTQLLYLGNLRKLENVCEDVTDINFDTKEKFLAGCLIAAFHEGKNLKKRYLEKKKGDNNSKLCKDLKYSFADYGDLIKGTSIWDNDFTKDLELNLQQIFGKLFRKYIKKKNISTEQDTSYSSLDELRESWWNTNKKYIWLAMKHGAGMNSTTCSCSGDSSSGENQTNSCDDIPTIDLIPQYLRFLQEWVEHFCEQRQAKVKDVITNCNSCKESGGTCNSDCEKKCKNKCDAYKTFIEDCKGVGGTGTAGSSWVKRWYQIYMRYSKYIEDAKRNRKAGTKSCGTSSTTNVSVSTDENKCVQS

SEQ ID NO: 3 - protein, 656 aa, Artificial
  Artificial construct
DYIKGDPYFAEYATKLSFILNSSDANNPSGETANHNDEVCNPNESEISSVGQAQTSDPSSNKTCNTHSSIKANKKKVCKHVKLGINNNDKVLRVCVIEDTSLSGVENCCFKDLLGILQENCSDNKSGSSSNGSCNNKNQEACEKNLEKVLASLTNCYKCDKCKSGTSTVNKNWIWKKSSGNKEGLQKEYANTIGLPPRTHSLYLGNLPKLENVCEDVKDINFDTKEKFLAGCLIAAFHEGKNLKKRYPQNKNDDNNSKLCKALEYSFADYGDLIKGTSIWDNEYTKDLELNLQQIFGKLFRKYIKKNISTEQDTLYSSLDELRESWWNTNKKYIWLAMKHGAGMNITTCCGDGSVTGSGSSCDDIPTIDLIPQYLRFLQEWVEHFCKQRQEKVKDVINSCNSCKNTSSKTKLGDTCNSDCEKKCKIECEKYKKFIEECRTAVGGTAGSSWSKRWDQIYKMYSKHIEDAKRNRKAGTKNCGITTGTISGESSGANSGVTTTENKCVQSDIDSFFKHLIDIGLTTPSSYLSIVLDDNICGDDKAPWTTYTTYTTYTTTEKCNKERDKSKSQQSNTSVVVNVPSPLGNTPHGYKYACQCKIPTNEETCDDRKEYMNQWISDTSKNPKGSGSTNNDYELYTYNGVKETKLPKKLNSPKLD

SEQ ID NO: 4 - protein, 643 aa, Artificial
  Artificial construct
DYIKDDPYSKEYTTKLSFILNSSDANTSSGETANHNDEACNCNESEISSVGQAQTSGPSSNKTCITHSFIKANKKKVCKDVKLGVRENDKVLRVCVIEDTSLSGVDNCCCQDLLGILQENCSDNKRGSSSNGSCNNKNQDECQKKLEKVFVSLTNGYKCDKCKSGTSTVNKKWIWKKSSGNEKGLQKEYANTIGLPPRTQSLYLGNLPKLGNVCEDVTDINFDTKEKFLAGCLIAAFHEGKNLKISHEKKKGDNGKKLCKALEYSFADYGDLIKGTSIWDNEYTKDLELNLQKAFGKLFGKYIKKNIASDENTSYSSLDELRESWWNTNKKYIWTAMKHGAEMNSTMCNADGSVTGSGSSCDDIPTTDFIPQYLRFLQEWVEHFCKQRQEKVNAVIENCNSCKNTSGERKIGGTCNGDCKTECKNKCEAYKNFIEDCKGGDGTAGSSWVKRWDQIYKRYSKHIEDAKRNRKAGTKSCGPSSITNASVSTDENKCVQSDIDSFFKHLIDIGLTTPSSYLSIVLDENNCGEDNAPWTTYTTYTTTEKCNKDKKKSKSQSCNTAVVVNVPSPLGNTPHEYKYACQCKIPTTEETCDDRKEYMNQWISDTSKKQKGSGSTNNDYELYTYTGVKETKLPKKLNSPKLD

SEQ ID NO: 5 - protein, 640 aa, Artificial
  Artificial construct
SYVKNDPYSKEYVTKLSFILNPSDANNPSGETANHNDEACNPNESEIASVGQAQTSDRLSQKACITHSFIGANKKIVCKDVKLGVREKDKDLKICVIEDDSLRGVENCCFKDLLGILQENCSDNKSGSSSNGSCNNKNQDECQKKLDEALASLHNGYKCDKCKSGTSRSKKIWTWRKFPGNGEGLQKEYANTIGLPPRTQSLYLGNLRKLENVCKGVTDINFDTKEKFLAGCLIAAFHEGKNLKISNKKKNDDNGKKLCKDLKYSFADYGDLIKGTSIWDNEYTKDLELNLQKIFGKLFRKYIKKNIASDENTLYSSLDELRESWWNTNKKYIWLAMKHGTTCSSGSGDNGDGSVTGSGSSCDDMSTIDLIPQYLRFLQEWVEHFCKQRQEKVKDVIENCKSCKNTSGERIIGGTCGSDCKTKCKGECDAYKNFIEECKRGDGTAGSPWSKRWDQIYMRYSKYIEDAKRNRKAGTKNCGTSSTTNAAENKCVQSDIDSFFKHLIDIGLTTPSSYLSIVLDENICGDDKAPWTTYTTYTTTEKCNKETDKSKSQSCNTAVVVNVPSPLGNTPHGYKYACECKIPTTEETCDDRKEYMNQWISDTSKKPKGGRSTNNDYELYTYNGVKETKLPKKSSSSKLD

SEQ ID NO: 6 - protein, 358 aa, Plasmodium falciparum
KCEKCKSEQSKKNNNIWIWRKFPGNGEGLQKEYANTIGLPPRTHSLYLGNLPKLENVCKDVKDINFDTKEKFLAGCLIAAFHEGKNLKTTYPQNKNADNNSKLCKDLKYSFADYGDLIKGTSIWDNDFTKDLELNLQKIFGKLFRKYIKKNIASDENTLYSSLDELRESWWNTNKKYIWLAMKHGAEMNSTMCNGDGSVTGSSDSGSTTCSGDNGSISCDDIPTIDLIPQYLRFLQEWVEHFCKQRQEKVKPVIENCKSCKNTSGERIIGGTCGSDCEKKCKGECDAYKKFIEECKGGGGGTGTAGSPWSKRWDQIYKRYSKYIEDAKRNRKAGTKSCGPSSTTNAAASTTESKCVQS

SEQ ID NO: 7 - protein, 333 aa, Plasmodium falciparum
KCDKCKSEQSKKNNKNWIWKQFPGNGEGLQKEYANTIGLPPRTHSLYLGNLPKLENVCKGVTDINFDTKEKFLAGCLIAAFHEGKNLKTSHEKKKGDNGKKLCKDLKYSFADYGDLIKGTSIWDNDFTKDLELNLQQIFGKLFRKYIKKNISAEQDTSYSSLDELRESWWNTNKKYIWLAMKHGTTCSSGSGDNGDGSVTGSGSSCDDMPTTDFIPQYLRFLQEWVEHFCKQRQEKVNAVITNCKSCKESGGTCNSDCEKKCKDECEKYKKFIEECRTAADGTAGSSWSKRWDQIYKMYSKHIEDAKRNRKAGTKNCGTSSTTNAAENKCVQS

SEQ ID NO: 8 - protein, 269 aa, Plasmodium falciparum
DYIKDDPYSKEYTTKLSFILNSSDANTSSGETANHNDEACNCNESEIASVEQASISDRSSQKAYITHSSIKTNKKKVCKYVKLGINNNDKVLRVCVIEDTSLSGVENCCFKDLLGILQENCSDNKRGSSFNDSCNNNNEEACQKKLEKVLASLTNGYKCEKCKSGTSRSKKKWIWKKSSGKEGGLQKEYANTIGLPPRTQSLYLGNLPKLENVCKGVTDINFDTKEKFLAGCLIAAFHEGKNLKPSHQNKNDDNNSKLCKDLKYSFADY

SEQ ID NO: 9 - protein, 333 aa, Plasmodium falciparum
KCDKCKSGTSRSKKKWTWRKSSGNKEGLQKEYANTIGLPPRTHSLYLGNLRKLENVCEDVTDINFDTKEKFLAGCLIAAFHEGKNLKTTYPQNKNDDNNSKLCKALKYSFADYGDLIKGTSIWDNDFTKDLELNLQKIFGKLFRKYIKKNISTEQHTSYSSLDELRESWWNTNKKYIWLAMKHGAEMNGTTCSCSGDSSDDIPTIDLIPQYLRFLQEWVEHFCKQRQAKVNAVINSCNSCKNTSGERKLGGTCGSECKTECKNKCDAYKEFIDGTGSGGGTGTAGSSWVKRWDQIYKRYSKYIEDAKRNRKAGSKNCGTSSTTNAAESKCVQS

SEQ ID NO: 10 - protein, 650 aa, Artificial
  Artificial construct
SYVKNNPYSAEYVTKLSFILNSSDANTSSETPSKYYDEVCNCNESEISSVGQAQTSGPSSNKTCITHSSIKTNKKKVCKDVKLGINNNDKVLRVCVIEDTSLSGVDNCCCQDLLGILQENCSDKNQSGSSSNGSCNNKNQDECQKKLEKVFASLTNGYKCDKCKSGTSRSKKKWIWRKSSGNEEGLQKEYANTIGLPPRTQSLYLGNLRKLENVCKGVTDINFDTKEKFLAGCLIAAFHEGKNLKTTYPQNKKKLCKDLKYSFADYGDLIKGTSIWDNEYTKDLELNLQKAFGKLFRKYIKKNISTEQHTLYSSLDELRESWWNTNKKYIWLAMKHGAGMNSTTCCGDGSVTGSGSSCDDIPTIDLIPQYLRFLQEWVEHFCKQRQEKVNAVIENCNSCKECGDTCNGECKTECEKKCKIECEKYKTFIEECVTAVGGTSGSPWSKRWDQIYKRYSKYIEDAKRNRKAGTKNCGITTGTISGESSGANSGVTTTENKCVQSDIDSFFKHLIDIGLTTPSSYLSIVLDDNICGADNAPWTTYTTYTTYTTTKNCDIKKKTPKSQPINTSVVVNVPSPLGNTPHGYKYACQCKIPTTEESCDDRKEYMNQWIIDTSKKQKGSGSTNNDYELYTYNGVKETKLPKKSSSSKLD

SEQ ID NO: 11 - protein, 643 aa, Artificial
  Artificial construct
SYVKDDPYSAEYVTKLSFILNSSDANTSSETPSKYYDEVCNCNESEISSVGQAQTSGPSSNKTCITHSSIKTNKKKVCKDVKLGINNNDKVLRVCVIEDTSLSGVDNCCCQDLLGILQENCSDKNQSGSSSNGSCNNKNQDECQKKLEKVFASLTNGYKCDKCKSGTSRSKKKWIWRKSSGNEEGLQKEYANTIGLPPRTQSLYLGNLPKLENVCKGVTDIIYDTKEKFLSGCLIAAFHEGKNLKTSHEKKNDDNGKKLCKALEYSFADYGDLIKGTSIWDNDFTKDLELNLQKIFGKLFRKYIKKNNTAEQDTSYSSLDELRESWWNTNKKYIWTAMKHGAGMNSTTCSGDGSVTGSGSSCDDMPTIDLIPQYLRFLQEWVEHFCKQRQEKVKDVITNCNSCKECGDTCNGECKTECKTKCKGECEKYKNFIEECNGTADGGTSGSSWSKRWDQIYKRYSKYIEDAKRNRKAGTKNCGTSSTTNAAASTTENKCVQSDIDSFFKHLIDIGLTTPSSYLSNVLDDNICGEDKAPWTTYTTYTTKNCDIQKKTPKPQSCDTLVVVNVPSPLGNTPHGYKYVCECKIPTTEETCDDRKEYMNQWIIDTSKKQKGSGSTNNDYELYTYNGVQIKQAAGTLKNSKLD

SEQ ID NO: 12 - protein, 269 aa, Plasmodium falciparum
NYIKGDPYSAEYATKLSFILNSSDTENASEKIQKNNDEVCNCNESEIASVEQAPISDRSSQKACITHSSIKANKKKVCKHVKLGVRENDKDLKICVIEDTSLSGVDNCCCQDLLGILQENCSDNKSGSSSNGSCNNNNEEICQKKLEKVLASLTNGYKCDKCKSGTSTVNKNWIWKKYSGKEGGLQEEYANTIGLPPRTQSLYLGNLPKLENVCEDVKDINFDTKEKFLAGCLIAAFHEGKNLKTSNKKKNDDNNSKLCKALKYSFADY

SEQ ID NO: 13 - protein, 344 aa, Plasmodium falciparum
KCDKCKSGTSTVNKKWIWKKYSGTEGGLQEEYANTIALPPRTQSLYLGNLPKLENVCKDVTDINFDTKEKFLAGCLIAAFHEGKNLKTTYLEKKKGDNGKKNDDNNSKLCKALKYSFADYGDLIKGTSIWDNDFTKDLELNLQQIFGKLFRKYIKKNIASDENTLYSSLDELRESWWNTNKKYIWLAMKHGAGMNSTMCNADGSVTGSGSSCDDIPTIDLIPQYLRFLQEWVEHFCKQRQAKVKDVITNCNSCKECGGTCNGECKTECEKKCKGECDAYKKFIEECKGKADEGTSGSSWSKRWDQIYKRYSKYIEDAKRNRKAGTKNCGPSSTTSTAESKCVQS

SEQ ID NO: 14 - protein, 334 aa, Plasmodium falciparum
KCDKCKSEQSKKNNNIWIWKKSSGTEGGLQKEYANTIALPPRTQSLYLGNLRKLENVCEDVKDINFDTKEKFLAGCLIAAFHEGKNLKKRYLEKKNGDNNSKLCKALKYSFADYGDLIKGTSIWDNEYTKDLELNLQKIFGKLFRKYIKKNNTAEQHTSYSSLDELRESWWNTNKKYIWLAMKHGTTCSSGSGDNGSISCDDIPTIDLIPQYLRFLQEWVEHFCEQRQGKVNAVIENCNSCKNTSSKTKLGGTCNGECKTECKGECDAYKEFIEKCKGTAAEGTSGSSWVKRWYQIYMRYSKYIEDAKRNRKAGTKNCGTSSTTSTAESKCVQS

SEQ ID NO: 15 - protein, 332 aa, Plasmodium falciparum
KCDKCKSEQSKKNNNIWIWKKSSGTEGGLQKEYANTIALPPRTQSLYLGNLRKLENVCEDVKDINFDTKEKFLAGCLIAAFHEGKNLKKRYLEKKNGDNNSKLCKALKYSFADYGDLIKGTSIWDNEYTKDLELNLQKIFGKLFRKYIKKNNTAEQDTSYSSLDELRESWWNTNKKYIWTAMKHGTTCSSGSGDNGSISCDDIPTIDLIPQYLRFLQEWVEHFCEQRQEKVKDVIKNCNSCKECGGTCNGECKTECKNKCKDECDAYKKFIEECEGKAAEGTSGSSWSKRWDQIYKRYSKYIEDAKRNRKAGTKNCGTSSTTSTAENKCVQS

SEQ ID NO: 16 - protein, 267 aa, Plasmodium falciparum
NYIKDDPYSAEYTTKLSFILNSSDTENASEKIQKNNDEVCNPNESGIACVELAQTSGSSSNKTCNTHSFIKANKKKVCKDVKLGINKKDKDLKICVIEDDSLRGVDNCCCQDLLGILQENCSDKNQSGSSSNGSCNNKNQEACQKKLENVFASLTNGYKCEKCKSEQSKKNNKNWIWKKYSVKEEGLQKEYANTIALPPRTQSLYLGNLPKLGNVCKGVTDINFDTKEKFLAGCLIAAFHEGKNLKTTYLQNKKKLCKALKYSFADY

SEQ ID NO: 17 - protein, 263 aa, Plasmodium falciparum
DYIKGDPYFAEYATKLSFILNSSDANTSSGETANHNDEACNPNESEIASVEQASISDRSSQKACNTHSSIKANKKKECKHVKLGVRENDKDLKICVIEDTSLSGVDNCCCQDLLGILQENCSDNKRGSSSNGSCDKNSEEICQKKLDEALASLHNGYKNQKCKSEQSKKNKNKWIWKKSSGNEKGLQKEYANTIGLPPRTQSLYLGNLPKLENVCEDVTDINFDTKEKFLAGCLIAAFHEGKNLKTTYPQNKNDDNGKKLCKD

SEQ ID NO: 18 - protein, 338 aa, Plasmodium falciparum
KCDKCKSEQSKKNNNIWIWKKSSGNKKGLQKEYANTIGLPPRTQSLYLGNLPKLENVCKDVTDINFDTKEKFLAGCLIAAFHEGKNLKISNEKKNDDNGKKLCKDLKYSFADYGDLIKGTSIWDNEYTKDLELNLQNNFGKLFRKYIKKNNTAEQHTLYSSLDELRESWWNTNKKYIWLAMKHGTTCSSGSGDNGDGSVTGSGSSCDDMSTIDLIPQYLRFLQEWVEHFCKQRQEKVNAVIENCNSCKNTSSKTKLGGTCNGECKTECEKKCKDECEKYKEFIEECKRGDGTAGSPWVKRWDQIYMRYSKYIEDAKRNRKAGTKSCGTSAAENKCVQS

SEQ ID NO: 19 - protein, 341 aa, Plasmodium falciparum
KCDKCKSEQSKKNNNIWIWKKSSGDEKGLQKEYANTIALPPRTQSLYLGNLPKLENVCKDVTDINFDTKEKFLAGCLIAAFHEGKNLKTSHQNKNADNGKKNDDNGKKLCKALKYSFADYGDLIKGTSIWDNEYTKDLELNLQQIFGKLFRKYIKRNNTAEQHTLYSSLDELRESWWNTNKKYIWLAMKHGTTCSSGSGDNGDGSVTGSGSSCDDMSTIDLIPQYLRFLQEWVEHFCKQRQEKVKDVITNCNSCKECGGTCGSDCKTKCEAYKKFIEECNGTADGGTSGSSWSKRWDQIYKRYSKYIEDAKRNRKAGTKNCGPSSGANSGVTTTENKCVQS

SEQ ID NO: 20 - protein, 352 aa, Plasmodium falciparum
KCEKCESEQSKKNNKYWIWKKSSGNGEGLQEEYANTIALPPRTHSLCLVCLHEKEGKKTQELKNIRTNSELLKERIIAAFHEGKNLKTSPQNKNDNGKKLCKDLKYSFADYGDLIKGTSIWDNEYTKDLELNLQKIFGKLFRKYIKKNNTAEQHTLYSSLDELRESWWNTNKKYIWLAMKHGAGMNSTMCNADGSVTGSSDSGSTTCCGDNGSISCDDMPTIDLIPQYLRFLQEWVEHFCEQRQEKVNAVITNCKSCKECGGTCNSDCEKKCKAYKEFIEKCKGGGTEGTSGSSWSKRWDQIYKRHSKHIEDAKRNRKAGTKNCGITTGTISGESSGANSGVTTTENKCVQS

SEQ ID NO: 21 - protein, 344 aa, Plasmodium falciparum
KCDKCKSGTSRSRKIWTWRKFRGNGEGLQKEYANTIGLSPRTQLLYLVCLHEKGKKTQELKNISTNSELLKEWIIAAFHEGKNLKTTYPQKKNDDNGKKLCKALKYSFADYGDLIKGTSIWDNDFTKDLELNLQKIFGKLFRKYIKKNIASDENTSYSSLDELRESWWNTNKKYIWTAMKHGAGMNGTTCCGDGSVTGSSDSGSTTCCGDGSVTGSGSSCDDIPTIDLIPQYLRFLQEWVEHFCEQRQEKVKDVITNCKSCKESEKKCKNKCDAYKEFIDGTGSGGGTGTAGSSWSKRWDQIYMRYSKYIEDAKRNRKAGTKNCGTSSGANSGVTTTENKCVQS

SEQ ID NO: 22 - protein, 350 aa, Plasmodium falciparum
KCEKCKSEQSKKNNKIWTWRKFPGNGEGLQKEYANTIGLSPRTQLLYLVCLHEKGKKTQHKTISTNSELLKEWIIAAFHEGKNLKKRYLEKKKGDNNSKLCKDLKYSFADYGDLIKGTSIWDNDFTKDLELNLQQIFGKLFRKYIKKNIASDENTSYSSLDELRESWWNTNKKYIWTAMKHGAGMNSTMCNGDGSVTGSSDSGSTTCSGDNGSISCDDIPTIDLIPQYLRFLQEWVEHFCEQRQEKVKDVIKNCNSCKECGGTCNGECKTECKNKCKDECEKYKNFIEVCTGGDGTAGSPWSKRWYQIYMRYSKYIEDAKRNRKAGTKSCGTSSGANSGVTTTESKCVQS

SEQ ID NO: 23 - protein, 359 aa, Plasmodium falciparum
KCEKCKSEQSKKNNKNWIWRKFPGNGEGLQKEYANTIGLPPRTHSLYLVCLHEKGKKTQELKNIRTNSELLKEWIIAAFHEGKNLKKRYHQNNNSGNKKKLCKALEYSFADYGDLIKGTSIWDNEYTKDLELNLQQIFGKLFRKYIKKNISTEQDTLYSSLDELRESWWNTNKKYIWLAMKHGAGMNSTTCCGDGSVTGSSDSGSTTCSGDNGSISCDDMPTIDLIPQYLRFLQEWVEHFCEQRQEKVKDVIENCKSCKNTSGERIIGGTCNGECKTECEKKCKAACEAYKTFIEECEGKAAEGTSGSSWSKRWYQIYMRYSKYIEDAKRNRKAGTKNCGKSSGANSGVTTTENKCVQS

SEQ ID NO: 24 - protein, 270 aa, Plasmodium falciparum
NYIKDDPYSKEYVTKLSFIPNSSDANNPSGETANHNDEVCNPNESEISSVEHAQTSVLLSQKAYITHSSIKANKKKVCKYVKLGVRENDKDLKICVIEDDSLRGVENCCFKDFLRILQENCSDNKRESSSNGSCNNNNEEACEKNLDEALASLTNCYKNQKCKSGTSTVNNNKWIWKKSSGKEGGLQKEYANTIGLPPRTQSLCLVVCLDEKEGKTQELKNIRTNSELLKEWIIAAFHEGKNLKKRYHQNKNDDNNSKLCKALKYSFADY

SEQ ID NO: 25 - protein, 334 aa, Plasmodium falciparum
KCDKCKSEQSKKNNKYWIWKKYSVKEGGLQKEYANTIALPPRTQSLCLVVCLDEKEGKTQELKNIRTNSELLKERIIAAFHEGKNLKTYHEKKKGDDGKKLCKDLKYSFADYGDLIKGTSIWDNDFTKDLELNLQKIFGKLFRKYIKKNNTAEQHTSYSSLDELRESWWNTNKKYIWTAMKHGAEMNGTTCSCSGDSSNDIPTIDLIPQYLRFLQEWVEHFCEQRQAKVNAVIKNCKSCKECGGTCNGECKTECKTKCKGECEKYKEFIEKCEGQAAEGTSGSSWSKRWYQIYMRYSKYIEDAKRNRKAGTKNCGTSSGANSGVTTTENKCVQS

SEQ ID NO: 26 - protein, 351 aa, Plasmodium falciparum
KCDKCKSEQSKKNNKNWIWKKYSGTEGGLQKEYANTIALPPRTQSLYLVCLHEKEEKTQELKNISTNSELLKEWIIAAFHEGKNLKISPQNKNDNGKNLCKDLKYSFADYGDLIKGTSIWDNDFTKDLELNLQQIFGKLFRKYIKKNNTAEQDTLYSSLDELRESWWNTNKKYIWTAMKHGAGMNGTTCCGDGSVTGSSDSGSTTCCGDGSVTGSGSSCDDIPTIDLIPQYLRFLQEWVEHFCEQRQAKVKDVIKNCNSCKECGGTCNGECKTECEKKCKGECEAYKKFIEKCNGGGGEGTSGSSWSKRWDQIYMRYSKYIEDAKRNRKAGTKNCGTSSTTNAAENKCVQS

SEQ ID NO: 27 - protein, 353 aa, Plasmodium falciparum
KCDKCKSGTSTVNKKWIWKKFPGKEGGLQEEYANTIALPPRTQSLCLVVCLDEKEGKTQHKTISTNSELLKEWIIAAFHEGKNLKISNKKKNDENNSKLCKDLKYSFADYGDLIKGTSIWDNDFTKDLELNLQKIFGKLFRKYIKKNNTAEQDTSYSSLDELRESWWNTNKKYIWLAMKHGTTCSSGSGDNGDGSVTGSSDSGSTTCCGDGSVTGSGSSCDDIPTIDLIPQYLRFLQEWVEHFCKQRQAKVKDVIENCKSCKNTSSKTKLGDTCNSDCKTKCKVACEKYKEFIEKCVSAAGGTSGSSWVKRWDQIYMRYSKYIEDAKRNRKAGTKNCGPSSTTSTAESKCVQS

SEQ ID NO: 28 - protein, 327 aa, Plasmodium falciparum
KCDKCKSGTSTVNKKWIWKKYSGKEGGLQKEYANTIGLPPRTQSLCLVCLHEKEGKTQELKNISTNSELLKEWIIAAFHEGKNLKISNKKKNDDNGKKLCKDLKYSFADYGDLIKGTSIWDNDFTKDLELNLQKIFGKLFRKYIKKNNTAEQDTLYSSLDELRESWWNTNKKYIWTAMKHGAGMNSTTCSCSGDSSNDIPTIDLIPQYLRFLQEWVEHFCKQRQEKVNAVITNCKSCKESGGTCNSDCEKKCKIECEKYKNFIEKCVTAAGGTSGSSWSKRWDQIYKMYSKYIEDAKRNRKAGTKNCGPSSTTNAAASTDENKCVQS

SEQ ID NO: 29 - protein, 628 aa, Artificial
  Artificial construct
NYIKGDPYFAEYATKLSFILNSSDTENASETPSKYYDEACNCNESEIASVGQAQTSGPSSNKTCITHSSIKTNKKKECKDVKLGINNNDKVLRVCVIEDTSLSGVDNCCCQDLLGILQENCSDNKRGSSSNGSCDKNSEEICQKKLEKVFASLTNGYKCDKCKSGTSRSKKKWIWKKSSGNEEGLQKEYANTIGLPPRTQSLCLVCLHEKEGKTQHKTISTNSELLKEWIIAAFHEGKNLKTSHEKKNDDNGKKLCKALEYSFADYGDLIKGTSIWDNEYTKDLELNLQKIFGKLFRKYIKKNNTAEQHTSYSSLDELRESWWNTNKKYIWTAMKHGAGMNGTTCSCSGDSSNDMPTIDLIPQYLRFLQEWVEHFCKQRQEKVNAVIENCNSCKESGGTCNSDCKTECKNKCEAYKEFIEDCKGGGTGTAGSPWSKRWDQIYKRYSKHIEDAKRNRKAGTKNCGTSSTTNAAASTDENKCVQSDVDSFFKHLIDIGLTTPSSYLSNVLDDNICGADKAPWTTYTTYTTTKNCDIQKKTPKSQSCDTLVVVNVPSPLGNTPHEYKYACECKIPTTEETCDDRKEYMNQWSCGSAQTVRGRSGKDDYELYTYNGVKETKPLGTLKNSKLD

SEQ ID NO: 30 - protein, 350 aa, Plasmodium falciparum
KCEKCKSEQSKKNNKNWIWRKFRGTEGGLQEEYANTIGLPPRTQSLCLVVCLDEKGKKTQELKNIRTNSELLKEWIIAAFHEGKNLKPSHQNKNSGNKENLCKALKYSFADYGDLIKGTSIWDNDFTKDLELNLQKIFGKLFRKYIKKNNTAEQHTSYSSLDELRESWWNTNKKYIWTAMKHGAEMNGTTCNADGSVTGSSDSGSTTCSGDNGSISCDDIPTIDLIPQYLRFLQEWVEHFCKQRQEKVNAVINSCNSCKNTSSKTKLGDTCNSDCKTKCKIECEKYKTFIEKCVTAAGGTSGSPWSKRWDQIYKRYSKYIEDAKRNRKAGTKNCGPSSTTSTAESKCVQS

SEQ ID NO: 31 - protein, 330 aa, Plasmodium falciparum
KCDKCKSEQSKKNNKNWIWRKYSGNGEGLQKEYANTIGLPPRTHSLYLVCLHEKEGKTQELKNIRTNSELLKEWIIAAFHEGKNLKTTYLENKNDENKKKLCKALKYSFADYGDLIKGTSIWDNDFTKDLELNLQKIFGKLFRKYIKKNIASDENTLYSSLDELRESWWNTNKKYIWTAMKHGAEMNGTTCSSGSGDNGSISCDDIPTIDLIPQYLRFLQEWVGHFCKQRQEKVNAVITNCNSCKESGGTCNSDCEKKCKIECEKYKKFIEECRTAAGGTSGSPWSKRWDQIYKMYSKYIEDAKRNRKAGTKNCGPSSTTSTAESKCVQS

SEQ ID NO: 32 - protein, 334 aa, Plasmodium falciparum
KCDKCKSEQSKKNNKNWIWRKYSGNGEGLQKEYANTIGLPPRTHSLYLVCLHEKEGKTQHKTISTNSELLKEWIIAAFHEGKNLKKRYPQNNNSGNKKKLCKDLKYSFADYGDLIKGTSIWDNEYTKDLELNLQKAFGKLFRKYIKKNIASDENTLYSSLDELRESWWNTNKKYIWLAMKHGAEMNGTMCNADGSVTGSGSSCDDMSTIDLIPQYLRFLQEWVEHFCEQRQAKVKDVINSCKSCKESGDTCNSDCEKKCKNKCDAYKTFIEEFCTADGGTAGSPWSKRWDQIYKRYSKYIEDAKRNRKAGTKNCGTSSGANSGVTTTENKCVQS

SEQ ID NO: 33 - protein, 350 aa, Plasmodium falciparum
KCDKCKSGTSTVNKNWIWKKYSGKEEGLQKEYANTIALPPRTHSLYLVCLHEKGKKTQELKNIRTNSELLKEWIIAAFHEGKNLKTSPQNNNSGNKKKLCKALKYSFADYGDLIKGTSIWDNDFTKDLELNLQKIFGKLFRKYIKKNNTAEQHTSYSSLDELRESWWNTNKKYIWLAMKHGAEMNGTTCCGDGSVTGSSDSGSTTCSGDNGSISCDDMPTTDFIPQYLRFLQEWVEHFCKQRQEKVKHVMESCKSCKECGDTCNGECKTECEKKCKNKCEAYKTFIEKCVSADGGTSGSSWSKRWDQIYMRYSKYIEDAKRNRKAGTKNCGTSSTTNAAASTAENKCVQS

SEQ ID NO: 34 - protein, 647 aa, Artificial
  Artificial construct
DYIKDDPYSAEYATKLSFILNPSDANTSSGETANHNDEVCNCNESEIASVELAPISDSSSNKTCITHSFIGANKKKECKDVKLGVREKDKDLKICVIEDDSLRGVENCCCQDLLGILQENCSDNKSGSSSNGSCDKNSEDECQKKLENVFASLKNGYKCDKCKSGTSTVNKKWIWRKYSGNGEGLQKEYANTIGLPPRTHSLYLVCLHEKEGKTQHKTISTNSELLKEWIIAAFHEGKNLKTSHQNNNSGNKKKLCKALKYSFADYGDLIKGTSIWDNDFTKDLELNLQKIFGKLFRKYIKKNIASDENTSYSSLDELRESWWNTNKKYIWLAMKHGAEMNSTMCNGDGSVTGSSDSGSTTCSGDNGSISCDDIPTIDLIPQYLRFLQEWVEHFCKQRQEKVKDVITNCKSCKESGDTCNSDCEKKCKNKCEAYKKFIEERRTAAQGTAESSWVKRWDQIYMRYSKYIEDAKRNRKAGTKSCGPSSTTNAAASTAENKCVQSDIDSFFKHLIDIGLTTPSSYLSIVLDDNICGADNAPWTTYTTYTTTKNCDIKKKTPKPQSCDTLVVVNVPSPLGNTPHEYKYACQCRTPNKQESCDDRKEYMNQWSSGSAQTVRGRSTNNDYELYTYNGVKETKPLGTLKNSKLD

SEQ ID NO: 35 - protein, 341 aa, Plasmodium falciparum
KCDKCKSGTSTVNKKWIWRKSSGNKEGLQKEYANTIGLPPRTQSLYLGNLPKLENVCEDVKDINFDTKEKFLAGCLIVSFHEGKNLKTSHEKKNDDNGKKLCKALEYSFADYGDLIKGTSIWDNEYTKDLELNLQKIFGKLFRKYIKKNNTAEQDTSYSSLDELRESWWNTNKKYIWTAMKHGAGMNITTCCGDGSSGENQTNSCDDIPTIDLIPQYLRFLQEWVEHFCKQRQEKVNAVVTNCKSCKESGGTCNGECKTKCKNKCEVYKTFIDNVGDGTAGSPWVKRWDQIYKRYSKHIEDAKRNRKAGTKNCGITTGTISGESSGATSGVTTTENKCVQS

SEQ ID NO: 36 - protein, 632 aa, Artificial
  Artificial construct
NYIKDDPYSKEYVTKLSFIPNSSDANTSSEKIQKNNDEVCNPNESGISSVEQAQTSGPSSNKTCITHSSIKANKKKECKDVKLGVRENDKDLKICVIEDTSLSGVDNCCCQDLLGILQENCSDNKRGSSSNDSCDNKNQDECQKKLDEALESLHNGYKNQKCKSGTSTVNKKWIWKKSSGNKEGLQKEYANTIGLPPRTQSLYLGNLPKLENVSKGVTDIIYDTKEKFLAGCLIVSFHEGKNLKTSHEKKNDDNGKKLCKALEYSFADYGDLIKGTSIWDNEYTKDLELNLQKAFGKLFRKYIKKNISAEQDTSYSSLDELRESWWNTNKKYIWIAMKHGAGMNGTTCCGDGSSGENQTNSCDDIPTIDLIPQYLRFLQEWVEHFCEQRQAKVKDVITNCKSCKNTSGERKIGGTCNGECKTKCKNKCEAYKTFIEHCKGGDGTAGSSWVKRWDQIYKRYSKHIEDAKRNRKAGTKSCGTSTAENKCVQSDIDSFFKHLIDIGLTTPSSYLSIVLDENNCGEDKAPWTTYTTTKNCDIQKDKSKSQSSDTLVVVNVPSPLGNTPHGYKYACQCKIPTTEETCDDRKEYMNQWSCGSARTMKRGYKNDNYELCKYNGVDVKPTTVRSSSTKLD

SEQ ID NO: 37 - protein, 639 aa, Artificial
  Artificial construct
DYIKGDPYSAEYVTKLSFIPNSSDANNPSEKIQKNNDEVCNCNESEISSVGQASISDPSSNKTCNTHSSIKANKKKVCKDVKLGVRENDKVLKICVIEHTSLRGVDNCCFKDLLGILQEPRIDKNQSGSSSNGSCDKNSEEACEKNLEKVLASLTNGYKCDKCKSGTSRSKKKWIWKKYSGKEGGLQEEYANTIGLPPRTQSLCLVVCLDEKEGKTQELKNISTNSELLKEWIIAAFPEGKNLKPSPEKKKGDNGKKLCKDLKYSFADYGDLIKGTSIWDNEYTKDLELNLQKIFGKLFRKYIKKNIASDENTLYSSLDELRESWWNTNKKYIWLAMKHGAGMNSTMCNADGSVTGSGSSCDDMPTIDLIPQYLRFLQEWVEHFCKQRQEKVKPVIENCNSCKNTSSERKIGGTCNSDCKTECKNKCEVYKKFIEDCKGGDGTAGSSWSKRWDQIYKRYSKYIEDAKRNRKAGTKNCGPSSTTNAAENKCVQSDIDSFFKHLIDIGLTTPSSYLSTVLDDNICGEDNAPWTTYTTYTTTKNCDKDKKKSKSQSCDTLVVVNVPSPLGNTPHEYKYACECRTPNKQESCDDRKEYMNQWISDNTKNPKGSGSGKDYYELYTYNGVDVKPTTVRSSSTKLD

SEQ ID NO: 38 - protein, 655 aa, Artificial
  Artificial construct
DYIKGDPYFAEYATKLSFILNSSDANTSSGETANHNDEACNCNESEISSVEHASISDPSSNKTCNTHSSIKANKKKVCKHVKLGVRENDKDLRVCVIEHTSLSGVENCCFKDFLRILQENCSDNKSGSSSNGSCDKNNEEACEKNLEKVFASLTNCYKCEKCKSEQSKKNNKKWTWRKSSGNKGGLQEEYANTIGLPPRTQSLCLVVCLDEKEGKKTQELKNIRTNSELLKEWIIAAFHEGKNLKPSHEKKNDDNGKKNDDNNSKLCKDLKYSFADYGDLIKGTSIWDNEYTKDLELNLQKIFGKLFRKYIKKNIASDENTLYSSLDELRESWWNTNKKYIWLAMKHGAEMNGTTCNADGSVTGSGSSCDDIPTIDLIPQYLRFLQEWVEHFCKQRQAKVKDVIENCKSCKESGNKCKTECKNKCEAYKKFIENCKGGDGTAGSSWVKRWDQIYMRYSKYIEDAKRNRKAGTKNCGPSSITNVSASTDENKCVQSDIDSFFKHLIDIGLTTPSSYLSIVLDDNICGDDKAPWTTYTTYTTYTTYTTYTTYTTYTTTKNCDKERDKSKSQSCNTAVVVNVPSPLGNTPHEYKYACECRTPSNKELCDDRKEYMNQWSSGSAQTVRDRSGKDYYELYTYNGVKETKLPKKLNSSKLD

SEQ ID NO: 39 - protein, 347 aa, Plasmodium falciparum
KCDKCKSEQSKKNNKYWIWKKSSVKEEGLQKEYANTIALPPRTHSLCLVVCLDEKGKKTQELKNISTNSELLKERIIAAFHEGKNLKTTYLEKKNADNNSKLCKALKYSFADYGDLIKGTSIWDNEYTKDLELNLQQIFGKLFRKYIKKNNTAEQHTLYSSLDELRESWWNTNKKYIWLAMKHGAGMNGTTCCGDGSVTGSSDSGSTTCSGDNGSISCDDMPTTDFIPQYLRFLQEWVEHFCKQRQEKVKDVIENCNSCKNNLGKTEINEKCKTECKNKCEAYKNFIEKFCTADGGTSGSPWSKRWDQIYKRYSKYIEDAKRNRKAGTKNCGTSSTTSTAENKCVQS

SEQ ID NO: 40 - protein, 335 aa, Plasmodium falciparum
KCEKCKSGTSTVNKYWIWRKSSGNKEGLQKEYANTIALPPRTHSLCLVVCLDEKEGKTQELKNISTNSELLKERIIAAFHEGENLKTSHEKKKGDDGKKNADNNSKLCKALKYSFADYGDLIKGTSIWDNEYTKDLELNLQKIFGKLFRKYIKKNIASDENTSYSSLDELRESWWNTNKKYIWLAMKHGAGMNGTTCSCSGDSSDDMPTTDFIPQYLRFLQEWVEHFCKQRQENVNAVIENCNSCKECGGTCNSDCEKKCKTECKNKCEAYKNFIEKFCTADGGTSGYSWSKRWDQIYKRYSKYIEDAKRNRKAGTKSCGTSSTTSTAESKCVQS

SEQ ID NO: 41 - protein, 667 aa, Artificial
  Artificial construct
SYVKNNPYSKEYVTKLSFILNPSDANNPSETPSKYYDEVCNCNESGIACVGQAQTSGPSSNKTCITHSFIGANKKKVCKDVKLGVREKDKDLKICVIEDTYLSGVDNCCFKDFLGMLQENCSDNKSGSSSNGSCNNKNQDECEKNLDEALASLTNGYKCEKCKSGTSTVNKYWIWRKSSGNKEGLQKEYANTIALPPRTHSLCLVVCLDEKEGKTQHKTISTNSELLKEWIIAAFHEGKNLKTSHEKKKGDDGKKNADNNSKLCKALKYSFADYGDLIKGTSIWDNDFTKDLELNLQKIFGKLFRKYIKKNIASDENTSYSSLDELRESWWNTNKKYIWLAMKHGAGMNSTTCCGDGSVTGSSDSGSTTCCGDGSVTGSGSSCDDMPTTDFIPQYLRFLQEWVEHFCKQRQENVNAVIENCNSCKECGGTCNSDCEKKCKTECKGECDAYKEFIEKCNGGAAEGTSGSSWSKRWDQIYKRYSKYIEDAKRNRKAGTKNCGTSSTTSTAESKCVQSDIDSFFKHLIDIGLTTPSSYLSIVLDENICGADNAPWTTYTTYTTYTTYTTTEKCNKETDKSKLQQCNTSVVVNVPSPLGNTPHGYKYVCECRTPNKQETCDDRKEYMNQWISDNTKNPKGSRSTNNDYELYTYNGVQIKPTTVRSNSTKLD

SEQ ID NO: 42 - protein, 348 aa, Plasmodium falciparum
KCDKCKSEQSKKNNKNWIWKKSSGNEKGLQKEYANTIGLPPRTQSLCLVVCLDEKEGKTQELKNIRTNSELLKEWIIAAFHEGKNLKTSHEKKKGDNNSKLCKDLKYSFADYGDLIKGTSIWDNEYTKDLELNLQNNFGKLFRKYIKKNIASDENTSYSSLDELRESWWNTNKKYIWLAMKHGAGMNSTTCSSGSGSTTCSSGSGSTTCSSGSGDSCDDMPTIDLIPQYLRFLQEWVEHFCKQRQEKVNAVIKNCNSCKESGGTCNGECKTECKNKCEAYKTFIEEFCTADGGTSGSPWSKRWDQIYKMYSKHIEDAKRNRKAGTKNCGPSSTTNVSVSTDENKCVQS

SEQ ID NO: 43 - protein, 652 aa, Artificial
  Artificial construct
DYIKDDPYFAEYVTKLSFILNSSDANNPSGETANHNDEVCNPNESGIASVEQAQTSDPSSNKTCNTHSSIKANKKKVCKHVKLGVRENDKDLKICVIEHTSLSGVENCCCQDFLRILQENCSDNKSGSSSNGSCNNKNQEACEKNLEKVLASLTNCYKCDKCKSEQSKKNNKNWIWKKSSGNEKGLQKEYANTIGLPPRTQSLCLVVCLDEKEGKTQELKNIRTNSELLKEWIIAAFHEGKNLKKRYPQNKNDDNNSKLCKDLKYSFADYGDLIKGTSIWDNEYTKDLELNLQNNFGKLFRKYIKKNISTEQDTLYSSLDELRESWWNTNKKYIWLAMKHGAGMNSTTCSSGSGSTTCSSGSGSTTCSSGSGDSCDDMPTTDFIPQYLRFLQEWVEHFCKQRQEKVNAVIKNCNSCKESGGTCNGECKTECKNKCEAYKTFIEEFCTADGGTSGSPWSKRWDQIYKMYSKHIEDAKRNRKAGTKNCGPSSTTNVSVSTDENKCVQSDIDSFFKHLIDIGLTTPSSYLSIVLDDNICGEDKAPWTTYTTYTTTKKCNKETDKSKSQSCNTAVVVNVPSPLGNTPHGYKYACECKIPTTEETCDDRKEYMNQWIIDTSKKQKGSGSGKDDYELYTYNGVDVKPTTVRSNSTKLD

SEQ ID NO: 44 - protein, 628 aa, Artificial
  Artificial construct
DYIKDDPYSAQYTTKLSFILNPSDANTSSEKIQKNNDEACNCNESGISSVGQAQTSGPSSNKTCITHSSIKANKKKVCKDVKLGINNNDKVLRVCVIEDTSLSGVDNCCCQDLLGILQENCSDNKRGSSSNGSCNNNNEEACEKNLDEAPASLHNGYKNQKCKSGTSRSKKKWIWKKSSGNEKGLQEEYANTIGLPPRTQSLCLVCLHEKEGKTQHKTISTNSELLKEWIIAAFHEGKNLKTSHEKKNDDNGKKLCKALEYSFADYGDLIKGTSIWDNEYTKDLELNLQKAFGKLFRKYIKKNNTAEQDTSYSSLDELRESWWNTNKKYIWIAMKHGAGMNGTTCSCSGDSSNDMPTIDLIPQYLRFLQEWVEHFCEQRQAKVKDVITNCKSCKESGNKCKTECKTKCKDECEKYKTFIEDCNGGGTGTAGSSWVKRWDQIYKRYSKHIEDAKRNRKAGTKNCGPSSITNAAASTDENKCVQSDIDSFFKHLIDIGLTTPSSYLSNVLDENSCGDDKAPWTTYTTYTTTKNCDIQKDKSKSQPINTSVVVNVPSPLGNTPYRYKYACECKIPTTEESCDDRKEYMNQWSCGSARTMKRGYKNDNYELCKYNGVDVKPTTVRSNSSKLD

SEQ ID NO: 45 - protein, 653 aa, Artificial
  Artificial construct
DYIKGDPYFAEYATKLSFILNPSDTENASETPSKYYDEACNPNESEIASVEQAQTSGPSSNKTCITHSSIKTNKKKECKDVKLGVRENDKDLKICVIEDTSLSGVDNCCFKDLLGILQENCSDNKRGSSSNDSCNNNNEEACEKNLDEALASLTNGYKCDKCKSGTSTVNKKWTWRKSSGNEEGLQKEYANTIGLPPRTQSLCLVCLHEKEGKTKHKTISTNSELLKEWIIAAFHEGKNLKTSHEKKNDDNGKKLCKALEYSFADYGDLIKGTSIWDNEYTKDLELNLQKAFGKLFRKYIKKNNTAEQDTSYSSLDELRESWWNTNKKYIWTAMKHGAEMNGTTCSSGSGDNGDSSITGSSDSGSTTCSGDNGSISCDDIPTTDFIPQYLRFLQEWVEHFCEQRQAKVKDVINSCNSCNESGGTCNGECKTKCKDECEKYKKFIEDCNGGDGTAGSSWVKRWDQIYKRYSKHIEDAKRNRKAGTKNCGPSSITNAAASTDENKCVQSDVDSFFKHLIDIGLTTPSSYLSIVLDENSCGDDKAPWTTYTTYTTTEKCNKERDKSKSQSSDTLVVVNVPSPLGNTPHEYKYACECKIPTNEETCDDRKDYMNQWISDTSKKQKGSGSGKDYYELYTYNGVQIKQAAGRSSSTKLD

SEQ ID NO: 46 - protein, 490 aa, Plasmodium falciparum
KCDKCKSEQSKKNNNKWIWKKYSGNGEGLQKEYANTIGLPPRTQSLCLVCLHEKEGKTQHKTISTNSELLKEWIIAAFHEGKNLKKRYPQNKNDDNNSKLCKALEYSFADYGDLIKGTSIWDNEYTKDLELNLQKAFGKLFRKYIKKNNTAEQDTSYSSLDELRESWWNTNKKYIWTAMKHGAEMNGTTCSSGSGDNGDSSCDDIPTIDLIPQYLRFLQEWVEHFCKQRQAKVKDVINSCNSCKNTSGERKIGGTCNSDCEKKCKVACDAYKTFIEECRTAVGGTAGSSWVKRWDQIYKRYSKHIEDAKRNRKAGTKNCGPSSTTNAAENKCVQSDIDSFFKHLIDIGLTTPSSYLSNVLDENSCGADKAPWTTYTTYTTYTTYTTYTTTEKCNKERDKSKSQQSNTSVVVNVPSPLGNTPHEYKYACECKIPTTEETCDDRKEYMNQWIIDNTKNPKGSGSTDNDYELYTYNGVQIKQAAGRSSSTKLD

SEQ ID NO: 47 - protein, 335 aa, Plasmodium falciparum
KCEKCKSGTSTVNNKWIWRKSSGKEGGLQKEYANTIGLPPRTQSLYLGNLPKLENVCKGVTDIIYDTKEKFLSGCLIAAFHEGKNLKTTYLEKKNDDNGKKLCKALEYSFADYGDLIKGTSIWDNEYTKDLELNLQKIFGKLFRKYIKKNNTAEQDTSYSSLDELRESWWNTNKKYIWIAMKHGAGMNGTTCSSGSGDSSNDIPTTDFIPQYLRFLQEWVENFCEQRQAKVKPVIENCNSCKESGGTCNGECKTKCKVACDAYKKFIDGTGSGGGSRPTGIAGSSWSKRWDQIYKRYSKHIEDAKRNRKAGTKNCGPSSITNVSVSTDENKCVQS

SEQ ID NO: 48 - protein, 637 aa, Artificial
  Artificial construct
NYIKDDPYSKEYVTKLSFIPNSSDANTSSEKIQKNNDEVCNPNESGISSVEQAQTSDPSSNKTCITHSSIKANKKKECKDVKLGVRENDKDLKICVIEHTSLSGVDNCCFKDFLRMLQEPRIDKNQRGSSSNGSCDKNSEEACEKNLDEALASLTNGYKCDKCKSEQSKKNNNKWIWKKFPGKEGGLQEEYANTIGLPPRTQYLCLVVCLDEKEGKTQELKNIRTNSELLKEWIIAAFHEGKNLKTTYPQKKNDDNGKKLCKDLKYSFADYGDLIKGTSIWDNEYTKNVELNLQNNFGKLFRKYIKKNNTAEQDTSYSSLDELRESWWNTNKKYIWLAMKHGAEMNSTTCCGDGSVTGSGSSCDDIPTIDLIPQYLRFLQEWVEHFCKQRQAKVKDVITNCNSCKESGNKCKTECKNKCKDECEKYKKFIEACGTAVGGTGTAGSPWSKRWDQIYKRYSKHIEDAKRNRKAGTKNCGPSSTTNAAENKCVQSDIDSFFKHLIDIGLTTPSSYLSIVLDDNICGADKAPWTTYTTYTTENCDIQKKTPKSQSCDTLVVVNVPSPLGNTPHGYKYACQCRTPNKQESCDDRKEYMNQWIIDNTKNPKGSGSGKDYYELCKYNGVKETKPLGTLKNSKLD

SEQ ID NO: 49 - protein, 330 aa, Plasmodium falciparum
KCDKCKSEQSKKNNNKWIWRKFPGKEGGLQKEYANTIGLPPRTQSLCLVCLHEKEGKTQHKTISTNSELLKEWIIAAFHEGKNLKTTYLEKKNAENKKKLCKALKYSFADYGDLIKGTSIWDNEYTKDLELNLQKIFGKLFRKYIKKNNTAEQDTSYSSLDELRESWWNTNKKYIWTAMKHGAGMNGTMCNADGSVTGSGSSCDDMPTTDFIPQYLRFLQEWVEHFCKQRQAKVKDVIENCKSCKESGNKCKTECKNKCDAYKTFIEECGTAVGGTAGSSWVKRWDQIYKRYSKHIEDAKRNRKAGTKNCGTSSTTNAAASTAENKCVQS

SEQ ID NO: 50 - protein, 269 aa, Plasmodium falciparum
NYIKDDPYSKEYVTKLSFILNSSDAENASETPSKYYDEACNCNESGISSVEQASISDRSSQKACNTHSFIGANKKKVCKHVKLGVRENDKDLKICVIEDDSLRGVENCCFKDFLRMLQEPRIDKNQRGSSSNDSCNNNNEEACEKNLDEALASLHNGYKNQKCKSEQSKKNNNKWIWKKSSGKEGGLQKEYANTIGLPPRTQSLCLVCLHEKEGKTQHKTISTNSELLKEWIIDAFHEGKNLKTTYLEKKKGDNGKKLCKALKYSFADY

SEQ ID NO: 51 - protein, 347 aa, Plasmodium falciparum
KCDKCKSEQSKKNNKNWIWKKSSGKEGGLQKEYANTIALPPRTQSLCLVVCLHEKEGKTQHKTISTNSELLKEWIIDAFHEGKNLKTTYLEKQNADNGKKNADNNSKLCKDLKYSFADYGDLIKGTSIWDNEYTKDLELNLQQIFGKLFRKYIKKNIASDENTLYSSLDELRESWWNTNKKYIWTAMKHGAEMNGTTCSSGSGDSSSGENQTNSCDDIPTIDLIPQYLRFLQEWVEHFCEQRQAKVKDVITNCKSCKESGGTCNSDCKTKCKGECEKYKKFIEKCKGGGTEGTSGSSWVKRWYQIYMRYSKYIEDAKRNRKAGTKSCGTSSGANSGVTTTESKCVQS

SEQ ID NO: 52 - protein, 269 aa, Plasmodium falciparum
DYIKDDPYSKEYTTKLSFILNSSDANTSSEKIQKNNDEVCNPNESEISSVEQAQTSRPSSNKTCITHSSIKANKKKVCKDVKLGVRENDKVLRVCVIEHTSLSGVENCCCQDLLGILQENCSDNKRGSSSNGSCDKNSEEACEKNLDEALASLTNCYKNQKCKSEQSKKNNNKWIWKKSSGNEKGLQKEYANTIGLPPRTQSLCLVCLHEKEGKTQELKNISTNSELLKEWIIAAFHEGKNLKTTYPQNKNDDNGKKLFKDLKYSFADY

SEQ ID NO: 53 - protein, 646 aa, Artificial
  Artificial construct
DYIKDDPYSKEYTTKLSFILNSSDANTSSEKIQKNNDEVCNPNESEISSVEQAQTSRPSSNKTCITHSSIKANKKKVCKDVKLGVRENDKVLRVCVIEHTSLSGVENCCCQDLLGILQENCSDNKRGSSSNGSCDKNSEEACEKNLDEALASLTNCYKNQKCKSEQSKKNNNKWIWKKSSGKEGGLQKEYANTIGLPPRTQSLYLGNLPKLENVCKGVTDINFDTKEKFLAGCLIAAFHEGKNLKTTYLEKKNDDNGKKLCKALEYSFADYGDLIKGTSIWDNEYTKDLELNLQKAFGKLFRKYIKKNNTAEQDTSYSSLDELRESWWNTNKKYIWTAMKHGAGMNGTTCSSGSGDSSNDIPTTDFIPQYLRFLQEWVENFCEQRQAKVKDVIENCNSCKNTSGERKIGDTCNSDCEKKCKDECEKYKKFIEDCKGGDGTAGSSWVKRWDQIYKRYSKHIEDAKRNRKAGTKNCGITTGTISGESSGATSGVTTTENKCVQSDIDSFFKHLIDIGLTTPSSYLSNVLDDNICGEDNAPWTTYTTYTTEKCNKETDKSKSQQSNTAVVVNVPSPLGNTPHGYKYACECKIPTTEETCDDRKEYMNQWSCGSAQTVRDRSGKDDYELCKYNGVQIKQAAGTLKNSKLD

SEQ ID NO: 54 - protein, 632 aa, Plasmodium falciparum
NYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLD

SEQ ID NO: 55 - protein, 2730 aa, Plasmodium falciparum
MDKSSIANKIEAYLGAKSDDSKIDQSLKADPSEVQYYGSGGDGYYLRKNICKITVNHSDSGTNDPCDRIPPPYGDNDQWKCAIILSKVSEKPENVFVPPRRQRMCINNLEKLNVDKIRDKHAFLADVLLTARNEGERIVQNHPDTNSSNVCNALERSFADIADIIRGTDLWKGTNSNLEQNLKQMFAKIRENDKVLQDKYPKDQNYRKLREDWWNANRQKVWEVITCGARSNDLLIKRGWRTSGKSNGDNKLELCRKCGHYEEKVPTKLDYVPQFLRWLTEWIEDFYREKQNLIDDMERHREECTSEDHKSKEGTSYCSTCKDKCKKYCECVKKWKSEWENQKNKYTELYQQNKNETSQKNTSRYDDYVKDFFKKLEANYSSLENYIKGDPYFAEYATKLSFILNSSDANNPSEKIQKNNDEVCNCNESGIASVEQEQISDPSSNKTCITHSSIKANKKKVCKHVKLGVRENDKDLRVCVIEHTSLSGVENCCCQDFLRILQENCSDNKSGSSSNGSCNNKNQEACEKNLEKVLASLTNCYKCDKCKSEQSKKNNKNWIWKKSSGKEGGLQKEYANTIGLPPRTQSLCLVVCLDEKGKKTQELKNIRTNSELLKEWIIAAFHEGKNLKPSHEKKNDDNGKKLCKALEYSFADYGDLIKGTSIWDNEYTKDLELNLQKIFGKLFRKYIKKNNTAEQDTSYSSLDELRESWWNTNKKYIWLAMKHGAGMNSTTCCGDGSVTGSGSSCDDIPTIDLIPQYLRFLQEWVEHFCKQRQEKVKPVIENCKSCKESGGTCNGECKTECKNKCEVYKKFIEDCKGGDGTAGSSWVKRWDQIYKRYSKYIEDAKRNRKAGTKNCGPSSTTNAAENKCVQSDIDSFFKHLIDIGLTTPSSYLSIVLDDNICGADKAPWTTYTTYTTTEKCNKETDKSKLQQCNTAVVVNVPSPLGNTPHGYKYACQCKIPTNEETCDDRKEYMNQWSCGSARTMKRGYKNDNYELCKYNGVDVKPTTVRSNSSKLDDKDVTFFNLFEQWNKEIQYQIEQYMTNTKISCNNEKNVLSRVSDEAAQPKFSDNERDRNSITHEDKNCKEKCKCYSLWIEKINDQWDKQKDNYNKFQRKQIYDANKGSQNKKVVSLSNFLFFSCWEEYIQKYFNGDWSKIKNIGSDTFEFLIKKCGNDSGDGETIFSEKLNNAEKKCKENESTNNKMKSSETSCDCSEPIYIRGCQPKIYDGKIFPGKGGEKQWICKDTIIHGDTNGACIPPRTQNLCVGELWDKRYGGRSNIKNDTKESLKQKIKNAIQKETELLYEYHDKGTAIISRNPMKGQKEKEEKNNDSNGLPKGFCHAVQRSFIDYKNMILGTSVNIYEYIGKLQEDIKKIIEKGTTKQNGKTVGSGAENVNAWWKGIEGEMWDAVRCAITKINKKQKKNGTFSIDECGIFPPTGNDEDQSVSWFKEWSEQFCIERLQYEKNIRDACTNNGQGDKIQGDCKRKCEEYKKYISEKKQEWDKQKTKYENKYVGKSASDLLKENYPECISANFDFIFNDNIEYKTYYPYGDYSSICSCEQVKYYEYNNAEKKNNKSLCHEKGNDRTWSKKYIKKLENGRTLEGVYVPPRRQQLCLYELFPIIIKNKNDITNAKKELLETLQIVAEREAYYLWKQYHAHNDTTYLAHKKACCAIRGSFYDLEDIIKGNDLVHDEYTKYIDSKLNEIFDSSNKNDIETKRARTDWWENEAIAVPNITGANKSDPKTIRQLVWDAMQSGVRKAIDEEKEKKKPNENFPPCMGVQHIGIAKPQFIRWLEEWTNEFCEKYTKYFEDMKSNCNLRKGADDCDDNSNIECKKACANYTNWLNPKRIEWNGMSNYYNKIYRKSNKESEDGKDYSMIMEPTVIDYLNKRCNGEINGNYICCSCKNIGENSTSGTVNKKLQKKETQCEDNKGPLDLMNKVLNKMDPKYSEHKMKCTEVYLEHVEEQLKEIDNAIKDYKLYPLDRCFDDKSKMKVCDLIGDAIGCKHKTKLDELDEWNDVDMRDPYNKYKGVLIPPRRRQLCFSRIVRGPANLRNLKEFKEEILKGAQSEGKFLGNYYNEDKDKEKALEAMKNSFYDYEYIIKGSDMLTNIQFKDIKRKLDRLLEKETNNTEKVDDWWETNKKSIWNAMLCGYKKSGNKIIDPSWCTIPTTETPPQFLRWIKEWGTNVCIQKEEHKEYVKSKCSNVTNLGAQESESKNCTSEIKKYQEWSRKRSIQWEAISEGYKKYKGMDEFKNTFKNIKEPDANEPNANEYLKKHCSKCPCGFNDMQEITKYTNIGNEAFKQIKEQVDIPAELEDVIYRLKHHEYDKGNDYICNKYKNINVNMKKNNDDTWTDLVKNSSDINKGVLLPPRRKNLFLKIDESDICKYKRDPKLFKDFIYSSAISEVERLKKVYGEAKTKVVHAMKYSFADIGSIIKGDDMMENNSSDKIGKILGDGVGQNEKRKKWWDMNKYHIWESMLCGYKHAYGNISENDRKMLDIPNNDDEHQFLRWFQEWTENFCTKRNELYENMVTACNSAKCNTSNGSVDKKECTEACKNYSNFILIKKKEYQSLNSQYDMNYKETKAEKKESPEYFKDKCNGECSCLSEYFKDETRWKNPYETLDDTEVKNNCMCKPPPPASNNTSDILQKTIPFGIALALGSIAFLFMKKKPKTPVDLLRVLDIPKGDYGIPTPKSSNRYIPYASDRYKGKTYIYMEGDTSGDDDKYIWDL

SEQ ID NO: 56 - protein, 2734 aa, Artificial
  Artificial construct
MDSTSTIANKIEEYLGAKSDDSKIDELLKADPSEVEYYRSGGDGDYLKNNICKITVNHSDSGKYDPCEKKLPPYDDNDQWKCQQNSSDGSGKPENICVPPRRERLCTYNLENLKFDKIRDNNAFLADVLLTARNEGEKIVQNHPDTNSSNVCNALERSFADLADIIRGTDQWKGTNSNLEKNLKQMFAKIRENDKVLQDKYPKDQKYTKLREAWWNANRQKVWEVITCGARSNDLLIKRGWRTSGKSDRKKNFELCRKCGHYEKEVPTKLDYVPQFLRWLTEWIEDFYREKQNLIDDMERHREECTREDHKSKEGTSYCSTCKDKCKKYCECVKKWKTEWENQENKYKDLYEQNKNKTSQKNTSRYDDYVKDFFEKLEANYSSLENYIKGDPYFAEYATKLSFILNPSDANNPSGETANHNDEACNCNESGISSVGQAQTSGPSSNKTCITHSSIKTNKKKECKDVKLGVRENDKDLKICVIEDTSLSGVDNCCCQDLLGILQENCSDNKRGSSSNDSCDNKNQDECQKKLEKVFASLTNGYKCDKCKSGTSRSKKKWIWKKSSGNEEGLQEEYANTIGLPPRTQSLYLGNLPKLENVCEDVKDINFDTKEKFLAGCLIVSFHEGKNLKKRYPQNKNSGNKENLCKALEYSFADYGDLIKGTSIWDNEYTKDLELNLQNNFGKLFGKYIKKNNTAEQDTSYSSLDELRESWWNTNKKYIWTAMKHGAEMNITTCNADGSVTGSGSSCDDIPTIDLIPQYLRFLQEWVENFCEQRQAKVKDVITNCKSCKESGNKCKTECKTKCKDECEKYKKFIEACGTAGGGIGTAGSPWSKRWDQIYKRYSKHIEDAKRNRKAGTKNCGTSSTTNAAASTDENKCVQSDIDSFFKHLIDIGLTTPSSYLSNVLDDNICGADKAPWTTYTTYTTTEKCNKERDKSKSQSSDTLVVVNVPSPLGNTPYRYKYACQCKIPTNEETCDDRKEYMNQWSCGSARTMKRGYKNDNYELCKYNGVDVKPTTVRSNSSKLDGNDVTFFNLFEQWNKEIQYQIEQYMTNANISCIDEKEVLDSVSDEGTPKVRGGYEDGRNNNTDQGTNCKEKCKCYKLWIEKINDQWGKQKDNYNKFRSKQIYDANKGSQNKKVVSLSNFLFFSCWEEYIQKYFNGDWSKIKNIGSDTFEFLIKKCGNNSAHGEEIFNEKLKNAEKKCKENESTDTNINKSETSCDLNATNYIRGCQSKTYDGKIFPGKGGEKQWICKDTIIHGDTNGACIPPRTQNLCVGELWDKSYGGRSNIKNDTKELLKEKIKNAIHKETELLYEYHDTGTAIISKNDKKGQKGKNDPNGLPKGFCHAVQRSFIDYKNMILGTSVNIYEHIGKLQEDIKKIIEKGTPQQKDKIGGVGSSTENVNAWWKGIEREMWDAVRCAITKINKKNNNSIFNGDECGVSPPTGNDEDQSVSWFKEWGEQFCIERLRYEQNIREACTINGKNEKKCINSKSGQGDKIQGACKRKCEKYKKYISEKKQEWDKQKTKYENKYVGKSASDLLKENYPECISANFDFIFNDNIEYKTYYPYGDYSSICSCEQVKYYKYNNAEKKNNKSLCYEKDNDMTWSKKYIKKLENGRSLEGVYVPPRRQQLCLYELFPIIIKNEEGMEKAKEELLETLQIVAEREAYYLWKQYNPTGKGIDDANKKACCAIRGSFYDLEDIIKGNDLVHDEYTKYIDSKLNEIFGSSDTNDIDTKRARTDWWENETITNGTDRKTIRQLVWDAMQSGVRYAVEEKNENFPLCMGVEHIGIAKPQFIRWLEEWTNEFCEKYTKYFEDMKSKCDPPKRADTCGDNSNIECKKACANYTNWLNPKRIEWNGMSNYYNKIYRKSNKESEGGKDYSMIMAPTVIDYLNKRCHGEINGNYICCSCKNIGAYNTTSGTVNKKLQKKETECEEEKGPLDLMNEVLNKMDKKYSAHKMKCTEVYLEHVEEQLNEIDNAIKDYKLYPLDRCFDDQTKMKVCDLIADAIGCKDKTKLDELDEWNDMDLRGTYNKHKGVLIPPRRRQLCFSRIVRGPANLRSLNEFKEEILKGAQSEGKFLGNYYKEHKDKEKALEAMKNSFYDYEDIIKGTDMLTNIEFKDIKIKLDRLLEKETNNTKKAEDWWKTNKKSIWNAMLCGYKKSGNKIIDPSWCTIPTTETPPQFLRWIKEWGTNVCIQKQEHKEYVKSKCSNVTNLGAQASESNNCTSEIKKYQEWSRKRSIRWETISKRYKKYKRMDILKDVKEPDANTYLREHCSKCPCGFNDMEEMNNNEDNEKEAFKQIKEQVKIPAELEDVIYRIKHHEYDKGNDYICNKYKNIHDRMKKNNGNFVTDNFVKKSWEISNGVLIPPRRKNLFLYIDPSKICEYKKDPKLFKDFIYWSAFTEVERLKKAYGGARAKVVHAMKYSFTDIGSIIKGDDMMEKNSSDKIGKILGDTDGQNEKRKKWWDMNKYHIWESMLCGYREAEGDTETNENCRFPDIESVPQFLRWFQEWSENFCDRRQKLYDKLNSECISAECTNGSVDNSKCTHACVNYKNYILTKKTEYEIQTNKYDNEFKNKNSNDKDAPDYLKEKCNDNKCECLNKHIDDKNKTWKNPYETLEDTFKSKCDCPKPLPSPIKPDDLPPQADEPFDPTILQTTIPFGIALALGSIAFLFMKVIYIYIYVCCICMYVCMYVCMYVCMYVCMYVCMHVCMLCVYVIYVFKICIYIEKEKRKK

SEQ ID NO: 57 - protein, 116 aa, Artificial Sequence
  Artificial construct
GAMVDTLSGLSSEQGQSGDMTIEEDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 58 - protein, 13 aa, Artificial Sequence
  Artificial Spytag sequence
AHIVMVDAYKPTK

SEQ ID NO: 59 - protein, 8 aa, Artificial Sequence
  Artificial Spytag sequence
AHIVMVDA

SEQ ID NO: 60 - protein, 10 aa, Artificial Sequence
  The beta-strand of CnaB2
ATHIKFSKRD

SEQ ID NO: 61 - protein, 104 aa, Artificial Sequence
  SpyLigase sequence
HHHHHHDYDGQSGDGKELAGATMELRDSSGKTISTWISDGQVKDFYLYPGKYTFVETAAPDGYEVATAITFTVNEQGQVTVNGKATKGGSGGSGGSGEDSATHI

SEQ ID NO: 62 - protein, 16 aa, Artificial Sequence
  isopeptide Spy0128 sequence
TDKDMTITFTNKKDAE

SEQ ID NO: 63 - protein, 282 aa, Artificial Sequence
  Split-Spy0128 sequence
ATTVHGETVVNGAKLTVTKNLDLVNSNALIPNTDFTFKIEPDTTVNEDGNKFKGVALNTPMTKVTYTNSDKGGSNTKTAEFDFSEVTFEKPGVYYYKVTEEKIDKVPGVSYDTTSYTVQVHVLWNEEQQKPVATYIVGYKEGSKVPIQFKNSLDSTTLTVKKKVSGTGGDRSKDFNFGLTLKANQYYKASEKVMIEKTTKGGQAPVQTEASIDQLYHFTLKDGESIKVTNLPVGVDYVVTEDDYKSEKYTTNVEVSPQDGAVKNIAGNSTEQETSTDKDMTI

SEQ ID NO: 64 - protein, 93 aa, Artificial Sequence
  SpyCatcher-delta-N sequence
EDSATHIKFSKRDEDGKELAGATMELRDSSGKTISTWISDGQVKDFYLYPGKYTFVETAAPDGYEVATAITFTVNEQGQVTVNGKATKGDAHI

SEQ ID NO: 65 - protein, 13 aa, Artificial Sequence
  Inversed spytaq sequence
KTPKYADVMVIHA

SEQ ID NO: 66 - protein, 134 aa, Artificial Sequence
  SdyCatcher_DANG_Short sequence
MGSSHHHHHHSSGLVPRGSHMASMTGGQQMGRGSSGLSGETGQSGNTTIEEDSTTHVKFSKRDANGKELAGAMIELRNLSGQTIQSWISDGTVKVFYLMPGTYQFVETAAPEGYELAAPITFTIDEKGQIWVDS

SEQ ID NO: 67 - DNA, 20 bp, Artificial Sequence
  DNA primer
tacaggccatgcacagagag

SEQ ID NO: 68 - DNA, 20 bp, Artificial Sequence
  DNA primer
tcttgagtgtgtggctttcg

SEQ ID NO: 69 - DNA, 20 bp, Artificial Sequence
  DNA primer
agtatcgacctcgtcggaag

SEQ ID NO: 70 - DNA, 20 bp, Artificial Sequence
  DNA primer
tcttgagtgtgtggctttcg

SEQ ID NO: 71 - DNA, 20 bp, Artificial Sequence
  DNA primer
agccctgatcactgacgaag

SEQ ID NO: 72 - DNA, 20 bp, Artificial Sequence
  DNA primer
tgcagagatgaacaggatgc

SEQ ID NO: 73 - DNA, 20 bp, Artificial Sequence
  DNA primer
aggaagactggggctagagg

SEQ ID NO: 74 - DNA, 20 bp, Artificial Sequence
  DNA primer
acctgtcaggacaaggtgga

SEQ ID NO: 75 - DNA, 20 bp, Artificial Sequence
  DNA primer
tacctccaaagcaccagagc

SEQ ID NO: 76 - DNA, 20 bp, Artificial Sequence
  DNA primer
aatctacccattcccgaacc

SEQ ID NO: 77 - DNA, 20 bp, Artificial Sequence
  DNA primer
tacctccaaagcaccagagc

SEQ ID NO: 78 - DNA, 20 bp, Artificial Sequence
  DNA primer
tgaggaggcatctggaaatc

(19) United States
(12) Patent Application Publication
(10) Pub. No.: US 2021/0278406 A1
(43) Pub. Date: Sep. 9, 2021

(54) ISOLATION OF CIRCULATING CELLS OF FETAL ORIGIN USING RECOMBINANT MALARIA PROTEIN VAR2CSA

(71) Applicant: VARCT DIAGNOSTIC APS, Copenhagen N (DK)

(72) Inventors: Ali Salanti, Copenhagen N (DK); Mads Daugaard, Copenhagen N (DK)

(21) Appl. No.: 17/259,815

(22) PCT Filed: Jul. 12, 2019
(86) PCT No.: PCT/EP2019/068898

§ 371 Date: Jan. 12, 2021

(30) Foreign Application Priority Data
Jul. 13, 2018 (EP) 18183421.9

(51) Int. Cl. G01N 33/569; G01N 33/68; C12N 5/073

(52) U.S. Cl. CPC G01N 33/56966; G01N 33/689; C12N 5/0605; G01N 2800/368; G01N 2333/445; G01N 2400/40; C12N 2503/00

(57) ABSTRACT

The present invention relates to functional binding fragments comprising the minimal CSA-binding fragments of VAR2CSA and their use in identification and isolation of circulating trophoblast and/or fetal cells suitable for non-invasive prenatal diagnostic testing. Thus, the present invention describes methods of identifying and isolating trophoblast and/or fetal cells in a biological sample such as a maternal blood, and utilizing this for prenatal diagnostics.

### FIELD OF THE INVENTION

The present invention relates to functional binding fragments comprising the minimal binding fragments of VAR2CSA and their use in identification and isolation of circulating trophoblast and/or fetal cells suitable for non-invasive prenatal diagnostic testing. Thus, the present invention describes methods of identifying and isolating trophoblast and/or fetal cells in a biological sample such as a maternal blood, and utilizing this for prenatal diagnostics.

### BACKGROUND OF THE INVENTION

Prenatal diagnostic testing involves the identification of chromosomal abnormalities and/or genetic diseases in a human fetus. The current practice for detecting chromosomal aberrations such as presence of extra chromosomes [e.g., the most common condition, Trisomy 21 (Down's syndrome); Klinefelter's syndrome (47, XXY); Trisomy 13 (Patau syndrome); Trisomy 18 (Edwards syndrome); 47, XYY; 47, XXX], absence of chromosomes [e.g., Turner's syndrome (45, X)], various translocations and deletions, as well as diagnosis of genetic diseases (e.g., Cystic Fibrosis, Tay-Sachs Disease) involves an invasive procedure, chorionic villus sampling (CVS) and/or amniocentesis (AC).

Because of the increased risk of Down's syndrome in pregnancies of advanced maternal age, prenatal screening was initially focused on women over the age of 35. However, due to the lack of prenatal screening in younger women, about 80% of Down syndrome infants were born to pregnant females under the age of 35. In recent years, prenatal screening has changed from being based on high maternal age to combined first-trimester screening for trisomy 21, followed by confirmation of aneuploidy in fetal or placental cells obtained by an invasive procedure. A national screening programme to detect chromosomal aberrations can dramatically decrease the number of children born with Down syndrome, however, only the most common aneuploidies are included in the screening programme.

CVS and amniocentesis are invasive procedures which carry procedure-related risks of miscarriage. Although the techniques of CVS and AC have been improved in recent years, and, when optimally performed, the risk of miscarriage is now considered to be low (0.2 and 0.1% respectively), the vast majority of foetuses exposed to this risk are healthy. Fetal or placental cells obtained by these procedures are either directly tested by FISH/DNA analyses, or expanded in culture and then subjected to karyotype analyses (e.g., by G-banding). Non-invasive prenatal diagnostics using maternal blood has been attempted. Although rare (e.g., one fetal cell per million nucleated maternal blood cells), fetal trophoblasts, leukocytes and nucleated erythrocytes were found in the maternal blood during the first trimester of pregnancy. However, the isolation of trophoblasts and leukocytes from the maternal blood is limited by the availability of fetal-specific antibodies. In addition, studies have shown that at least 50% of the nucleated red blood cells (NRBCs) isolated from the maternal blood are of maternal origin and moreover, certain cell types tend to persist in the maternal circulation and therefore potentially interfere with diagnosis of subsequent pregnancies (Bianchi D 1996, Troeger C, et al., 1999; Guetta et al., 2004).

Circulating trophoblasts have with varying success been isolated from maternal blood by several different methods related to surface antigen expression levels distinct from normal white blood cells. Published methods use an epithelial (EpCAM or CK) or an endothelial marker (CD105) to isolate cells (Hou S et al, 2017, Breman A M et al, 2016, Huang C E et al, 2017, Kolvraa S et al, 2016). Trophoblast cells leaving the placental cell columns to invade the uterus undergo epithelial to mesenchymal transition resulting in loss of epithelial markers (Santisebastian M et al., 2009, Zhou Y et al, 1997). This transition with a shift in protein expression from epithelial to mesenchymal markers may lead to failure to identify circulating trophoblast cells if using an epithelial or endothelial marker for identification of trophoblasts.

The malaria parasite Plasmodium falciparum utilizes host cell proteoglycans in almost all stages of its complex life cycle. The sporozoite infects hepatocytes in the liver through surface-expressed circumsporozoite protein interacting with highly sulfated heparan sulfate proteoglycans (HSPG). Merozoite infection of the erythrocytes is mediated by EBA-175 binding to sialic acid on glycophorin A. In addition, a number of Plasmodium falciparum Erythrocyte Membrane Protein 1 (PfEMP1) proteins, mediating host endothelial adhesion, have been described as glycan-binding. One of these is VAR2CSA. VAR2CSA binds with high affinity to a specific type of chondroitin sulfate A (CSA) attached to proteoglycans, so called Chondroitin Sulfate Proteoglycans (CSPG).

### OBJECT OF THE INVENTION

It is an object of embodiments of the invention to provide methods for detection for the presence, optionally also isolation, of circulating trophoblast and/or fetal cells.

It is an object of embodiments of the invention to provide methods of testing for pregnancy or for fetal diagnostics, such as non-invasive fetal diagnostics.

It is an object of embodiments of the invention to provide methods of testing for a trophoblastic disease, such as extrauterine pregnancy and gestational trophoblastic disease.

### SUMMARY OF THE INVENTION

It has been found by the present inventors that recombinant VAR2CSA binds with high affinity and specificity to placental CSA chains.

Trophoblasts present an attractive target cell type for non-invasive prenatal diagnosis since they can be isolated from a maternal blood in the first trimester, are distinguishable from maternal blood cells due to their unique structure, and are absent in normal adult blood. Trophoblasts are formed from the outer layer of the blastocyst that provide nutrients to the embryo and are the first cells to differentiate from the fertilized egg. Trophoblast cells form a large part of the placenta.

VAR2CSA binds with high affinity to a specific type of chondroitin sulfate A (CSA) attached to proteoglycans, so called Chondroitin Sulfate Proteoglycans (CSPG), abundantly present in particular in the syncytiotrophoblast layer of the human placenta on the maternal side. VAR2CSA is a large multi-domain protein (350 kDa) expressed on the surface of P. falciparum-infected erythrocytes (IEs), and the VAR2CSA-CSA interaction is responsible for placenta specific sequestration in placental malaria (PM). Importantly, recombinant VAR2CSA has shown affinity for CSA in the low nano-molar range, and high specificity towards placental type of CSA with no binding to CSA present in other tissues in the human body.

Epidemiological studies show that VAR2CSA expressing parasites exclusively bind in the placenta despite the fact that CSA is present in other tissues and on other cells. In line with this, the present inventors have found that recombinant VAR2CSA (rVAR2) only binds trophoblast cells, in particular syncytiotrophoblast with no binding to other CSA expressing cells or CSA expressing tissues. This entails that the sulfation pattern of CSA in the placental is unique and that rVAR2 through evolution has been optimized to exclusively bind placental CSA and not normal CSA.

In the present invention we show that circulating trophoblast and/or fetal cells express this distinct placental type CSA and that recombinant VAR2CSA can readily distinguish a trophoblast cell from normal white blood cells. We have developed a technology using recombinant VAR2CSA to isolate single and rare trophoblast and/or fetal cells from a blood sample of a pregnant woman enabling non-invasive prenatal diagnostics.

Accordingly, the present inventors suggest using this specific and high affinity binding between VAR2CSA and CSA for detecting, purifying, and/or isolating circulating trophoblast and/or fetal cells.

The first aspect, the present invention relates to a method for the identification of a trophoblast cell in a biological sample, the method comprising:
a) contacting a biological sample comprising trophoblast and/or fetal cells expressing CSA with a VAR2CSA polypeptide, or a conjugate or fusion protein thereof;
b) detecting said VAR2CSA polypeptide or conjugate or fusion protein thereof specifically bound to said trophoblast and/or fetal cells expressing CSA.

The second aspect the present invention relates to a method of testing for pregnancy in a female subject, the method comprising identifying in a biological sample of the subject a trophoblast and/or fetal cell according to the methods of the invention, wherein a presence of said trophoblast and/or fetal cell is indicative of the pregnancy in the subject.

The third aspect the present invention relates to a method of testing a female subject for a trophoblastic disease, such as extrauterine pregnancy and gestational trophoblastic disease, the method comprising:
(a) identifying and isolating in a biological sample of a pregnant female a trophoblast and/or fetal cell according to the method of the invention; and
(b) subjecting said trophoblast and/or fetal cell to an assay specific to said trophoblastic disease, thereby diagnosing the disease.

A further aspect the present invention relates to a method of prenatally diagnosing or examining a conceptus, comprising
(a) identifying and isolating in a biological sample of a pregnant female a trophoblast and/or fetal cell according to the method of the invention; and
(b) subjecting said trophoblast and/or fetal cell to a conceptus diagnostic assay, thereby prenatally diagnosing the conceptus.

The diagnosing or examining of a conceptus is to be understood in a broad sense and may include a genetic analysis, such as a chromosomal analysis, e.g. to determine the sex of the conceptus, a genetic fingerprint, specific genetic mutations and polymorphisms.

In some embodiments these methods further comprising culturing the trophoblast and/or fetal cells prior to step (b) under conditions suitable for proliferation of the trophoblast.

In a further aspect the present invention relates to a method of generating a trophoblast and/or fetal cell culture, comprising:
(a) isolating trophoblast and/or fetal cells according to the method of the invention, and
(b) culturing said trophoblast and/or fetal cells under conditions suitable for proliferation of said trophoblasts, thereby generating the trophoblast culture. In some embodiments this conceptus diagnostic assay is effected by a chromosomal analysis.

In some embodiments this diagnosing the conceptus comprises identifying at least one chromosomal and/or DNA abnormality, and/or determining a paternity of the conceptus.

In a further aspect the present invention relates to a diagnostic composition comprising a VAR2CSA polypeptide bound to at least one trophoblast cell.

In a further aspect the present invention relates to a method of treating a female subject for a trophoblastic disease, such as extrauterine pregnancy and gestational trophoblastic disease, the method comprising
(a) identifying and isolating in a biological sample of a pregnant female a trophoblast cell according to the method of the invention;
(b) subjecting said trophoblast cell to an assay specific to said trophoblastic disease, thereby diagnosing the disease; and

c) administering a treatment to said female subject, wherein said treatment is specific for said disease diagnosis.

In a further aspect the present invention relates to a method of treating a female subject for a trophoblastic disease, such as extrauterine pregnancy and gestational trophoblastic disease, the method comprising:

(a) ordering a test for diagnosing a trophoblastic disase, wherein said test comprises
(i) identifying and isolating in a biological sample of a pregnant female a trophoblast cell according to the method of the invention;
   (ii) subjecting said trophoblast cell to an assay specific to said trophoblastic disease, thereby diagnosing the disease; and
(b) administering a treatment to said female subject, wherein said treatment is specific for said disease diagnosis.

### LEGENDS TO THE FIGURE

FIG. 1: Flow cytometry analysis of human trophoblast BeWo cells in a blood sample.

FIG. 2: Immunofluorescence microscopy analysis of human trophoblast BeWo cells (orange) in a blood sample. Cell nuclei (blue).

FIG. 3: Immunohistochemistry staining of fetal and placental tissue. (A) rVAR2 binding to placental syncytiotrophoblast cells. (B) rVAR2 binding to fetal primitive gut and hand tissue.

FIG. 4: NESTED-PCR on DNA purified from VAR2-captured circulating trophoblasts/fetal cells in pregnant women's blood samples (1st trimester) using Y-chromosome specific primers. Yellow box designates positive detection of the Y-chromosome gene DYS14 in Pregnant Woman 1 (#PW1).

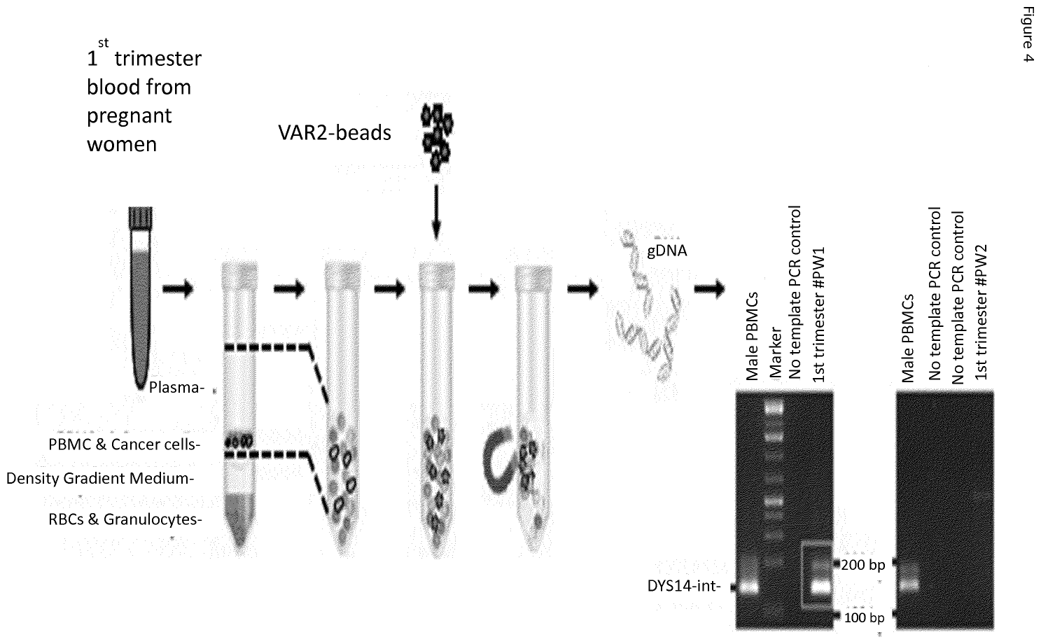

### DETAILED DISCLOSURE OF THE INVENTION

Definitions

As used herein the term “trophoblast” refers to a cell of the outer layer of a blastocyst derived from the placenta and formed during the first stage of pregnancy and the first cells to differentiate from the fertilized egg developing into a mammalian embryo or fetus. The trophoblast proliferates and differentiates into three types of trophoblast cells in the placental tissue: the cytotrophoblast, the syncytiotrophoblast, and the intermediate trophoblast, and as such, the term “trophoblast” as used herein encompasses any of these cells.

As used herein the term “a trophoblastic disease” refers to a disease or inappropriate condition involving trophoblasts. The term includes extra-uterine or ectopic pregnancy, as well as pregnancy-related tumours, such as benign tumour hydatidiform mole, such as complete hydatidiform mole, and partial hydatidiform mole, as well as malignant tumours including invasive mole, choriocarcinoma, placental site trophoblastic tumours, epithelioid trophoblastic tumours, and choriocarcinoma.

As used herein the term “fetal cell” or “cells of fetal origin” refers to any part at any differential cell stage of a fertilized oocyte developing from the inner cell mass or embryoblast of the blastocyst and into cells of the fetus. The term “fetal cells” include embryonic stem cells including human stem cells, totipotent and pluripotent stem cells that are able to develop into any type of cell, including those of the placenta, as well as any cells giving rice to the definitive structures of the fetus.

In some embodiments the term “trophoblast and/or fetal cell” as used herein refers to a trophoblast cell. In some embodiments the term “trophoblast and/or fetal cell” as used herein refers to a cell of the inner cell mass (embryoblast) of the blastocyst. In some embodiments the term “trophoblast and/or fetal cell” as used herein refers to an embryonic stem cell. In some embodiments the term “trophoblast and/or fetal cell” as used herein refers to a cell of the fetal placenta (Chorion frondosum).

A biological sample used by the methods of the invention can be a blood sample, such as a peripheral blood sample, a transcervical sample, an intrauterine sample or an amniocyte sample derived from a pregnant female subject at any stage of gestation. In some embodiments the biological sample is not placenta. The biological sample may be obtained using invasive or non-invasive methods. A maternal blood sample can be obtained by drawing blood from a peripheral blood vessel (e.g., a peripheral vein), or from any other blood vessel such as the uterine vein. The blood sample can be of about 20-25 ml. According to some embodiments of the invention, the peripheral blood sample is obtained during the first trimester of pregnancy (e.g., between the 6-13 weeks of gestation).

Thus, according to an aspect of some embodiments of the invention, there is provided a method of testing a pregnancy in a subject. The method is effected by identifying in a biological sample of the subject a trophoblast according to the teachings described herein, wherein a presence of the trophoblast in the biological sample is indicative of the pregnancy in the subject.

The biological sample used according to this aspect of the invention can be a blood sample, a transcervical sample or an intrauterine sample. According to some embodiments of the invention, an expression level of the trophoblast marker above a predetermined threshold is indicative of the pregnancy in the subject. The predetermined threshold can be determined according to reference samples obtained from non-pregnant control subjects. Once identified, the trophoblast can be further isolated from the biological sample.

According to some aspects the invention relates to a method of isolating a trophoblast from a biological sample. The method is effected by identifying the trophoblast in the biological sample according to the teachings described herein and optionally isolating the trophoblast, thereby isolating the trophoblast from the biological sample. As used herein the phrase “isolating a trophoblast” refers the physical isolation of a trophoblast cell from a heterogeneous population of cells (e.g., the other cells within the trophoblast-containing biological sample). According the present invention provides methods for testing a pregnancy in a subject.

The present invention further allows for prenatal diagnosis of a conceptus. As used herein the term “conceptus” refers to an embryo, a fetus or an extraembryonic membrane of an ongoing pregnancy. The method of prenatally diagnosing a conceptus is effected by identifying in a biological sample of a pregnant female a trophoblast as described herein, and subjecting the sample containing trophoblast to a conceptus diagnostic assay.

A conceptus diagnostic assay can be performed directly on the trophoblast (whether isolated from the trophoblast-containing sample or not), or can be performed on cultured trophoblast cells. The conceptus diagnostic assay can be effected by a chromosomal analysis in order to determine abnormalities on a chromosome level or by DNA analysis, such as to identify changes in DNA sequences (as compared to normal controls) which can be related to diseases or genetic polymorphisms.

VAR2CSA Polypeptides

The present invention is based on the fact that a part of a malaria protein, the so-called VAR2CSA, can bind to an extra-cellular CSA-conjugated CSPG with very high specificity and very high binding strength. This part of the technology is described in WO2013117705 A1.

It is to be understood that for a protein comprising a VAR2CSA polypeptide, any VAR2CSA sequences and polypeptides as defined herein may be used. Accordingly, this aspect is not limited to the use of minimal binding fragments. This applies whenever the term VAR2CSA sequence or polypeptide is used.

CSA interacts with many important factors such as growth hormones, cytokines, chemokines, and adhesion molecules and is thought to be involved in structural stabilization, cytokinesis, cell proliferation, differentiation, cell migration, tissue morphogenesis, organogenesis, infection, and wound repair. CS chains are composed of alternating units of N-acetyl-D-galactosamine (GalNAc) and glucuronic acid residues. Glucuronic acid can be sulfated at its C2 position and GalNAc can be sulfated at C4 and/or C6, giving rise to various disaccharide units. Varying modifications of the sugar backbone allows structural and functional heterogeneity of the CS chains. Placenta adhering P. falciparum parasites specifically associate with CS chains that are predominantly C4 sulfated.

Recombinant VAR2CSA protein has been shown to bind with unprecedented high affinity and specificity to CSA. This may be due to an interaction with CSA that is not only dependent on the charged sulfates but also on the CS backbone.

The inventors of the present invention have identified a malaria protein, VAR2CSA, which binds CSA in the intervillous space of the placenta with an affinity below 10 nM. Smaller recombinant parts of VAR2CSA have been produced at high yields that bind CSA with characteristics similar to that of the full-length and native VAR2CSA protein. The recombinant VAR2CSA protein does not bind other types of CS such as chondroitin sulfate C (C6S) or highly sulfated GAGs such as heparan sulfate (HS). Recombinant proteins can be produced to bind with high affinity to CSA in various expression systems, here among S2 cells, T.Ni cells, CHO cells and E. coli strains including BL21 and Shuffle cells (tm New England Biolabs).

The inventors of the present invention have also identified a single small (75 kDa) antigen that binds CSA with very high affinity (nM) and high specificity.

This VAR2CSA recombinant protein binds strongly at low concentrations to a wide range of cancer cell lines expressing placental-like CSA. As a control molecule another VAR2CSA protein was used, which is identical to the minimal binding VAR2CSA construct except for a 151 amino acids truncation in the C-terminal part of the molecule. This truncation removes the CSA binding. Recombinant VAR2CSA protein fails to interact with human red blood cells and peripheral blood mononuclear cells (PBMC).

The advantages of targeting CSA on circulating trophoblast with VAR2CSA over other current proteins in development are numerous:

1) The interaction between VAR2CSA and CSA is of unprecedented high affinity and highly specific.

2) VAR2CSA is a stable protein that is well characterized and can be highly expressed in organisms compatible with large-scale protein production.

The inventors of the present invention have shown that recombinant VAR2CSA binds specifically to placental derived cell lines by flow cytometry. Example 1 shows VAR2CSA binding to human trophoblast cells (HTR8) in vitro. Example 2 shows BeWo mixed into a blood sample and how we specifically using VAR2CSA can identify the trophoblast cells from normal white blood cells. Using immunohistochemistry staining with VAR2CSA on pre-term placentas we demonstrate that VAR2CSA specifically binds trophoblast cells (Example 3). Using immunohistochemistry staining with VAR2CSA on human fetus we demonstrate that VAR2CSA specifically binds fetal cells from the embryo (Example 3). We advise methods to identify rare trophoblast cells in a blood sample using affinity capture with VAR2CSA coated magnetic beads, or whole blood analyses followed by VAR2CSa staining and scanning microscopy (Example 4), such cells can be picked by a picking device, such as a CellCelector for genetic analyses (Example 5). Even without preselection can rVAR2 be used to identify trophoblast cells from blood samples (Example 6). Using blood samples from pregnant women, we demonstrate that we can identify and isolate trophoblast cells for genetic analyses (Example 7).

The terms “VAR2CSA polypeptide” as used herein refers to the extracellular part of a specific Erythrocyte Membrane Protein 1 (PfEMP1) protein expressed by Plasmodium falciparum interacting with chondroitin sulfate proteoglycans (CSPG) and characterized by having a sequence of SEQ ID NO:55 or SEQ ID NO:56 or fragments or variants thereof with the ability to bind chondroitin sulfate A (CSA) that could be presented on a proteoglycans (CSPG).

In some embodiments, the VAR2CSA polypeptide according to the present invention at least comprises the protein fragment of VAR2CSA, which fragment consist of or comprises a sequential amino acid sequence of a) ID1, and b) DBL2Xb. Sometimes the term “a recombinant VAR2CSA protein”, “a recombinant VAR2CSA polypeptide” or “recombinant VAR2CSA” (rVAR2) may be used interchangeably with the terms “VAR2CSA polypeptide”.

It is to be understood that a suitable VAR2CSA polypeptide to be used according to the invention may be a non-natural form, i.e. a fragment of the natural form representing only the extracellular part of the natural protein, or an even smaller fragment comprising only the minimal binding domains.

In some embodiments a “recombinant VAR2CSA” or “rVAR2” refers to a recombinant fragment of a VAR2CSA polypeptide comprising at least the minimal binding domains two binding domains ID1-DBL2Xb, or the domains ID1-ID2a. In specific embodiments rVAR2 comprises at least ID1 and DBL2Xb or ID1, DBL2Xb and ID2a.

In some embodiments, the VAR2CSA polypeptide according to the present invention at least comprises the minimal binding domains, which is a fragment containing at least ID1-DBL2Xb, or ID1-ID2a.

In some embodiments, the VAR2CSA polypeptide according to the present invention at least comprises the protein fragment of VAR2CSA, which fragment consist of or comprises a sequential amino acid sequence of a) ID1, and b) DBL2Xb, and c) ID2a.

In some embodiments, the VAR2CSA polypeptide according to the present invention comprises a protein fragment of VAR2CSA defined by amino acids 1-384 of SEQ ID NO:55 or amino acids 1-385 of SEQ ID NO:56 (also referred to as DBL1X), or a C-terminal amino acid sequence thereof, such as at least the 20, 40, 60, 80, 100, 120, 140, 160, 180, 200, 220, 240, 260, 280, 300, 320, 340, 360, or 380 C-terminal amino acids of this this fragment of VAR2CSA, or any functional variant thereof. It is to be understood that this amino acid sequence will be linked by its C-terminal to the N-terminal part of the VAR2CSA polypeptide defined as ID1.

Included within the definition of a VAR2CSA polypeptide is polypetides described in Salanti A. et al Mol. Micro 2003 July; 49(1):179-91, in Khunrae P. et al, 3 Mol Biol. 2010 Apr. 2; 397(3):826-34, in Srivastava A. et al, Proc Natl Acad Sci USA. 2010 Mar. 16; 107(11):4884-9, in Dahlbäck M. et al, J Biol Chem. 2011 May 6; 286(18):15908-17, or in Srivastava A. et al, PLoS One. 2011; 6(5):e20270.

The term “ID1” as used herein refers to a domain of VAR2CSA characterized by having an amino acid sequence with at least 70%, at least 80%, at least 85%, at least 90%, at least 91%, at least 92%, at least 93%, at least 94%, at least 95%, at least 96%, at least 97%, at least 98%, at least 99% or more sequence identity to an amino acid sequence identified by 1-152 of SEQ ID NO:1.

The term “DBL2Xb” as used herein refers to a domain of VAR2CSA characterized by having an amino acid sequence with at least 70%, at least 80%, at least 85%, at least 90%, at least 91%, at least 92%, at least 93%, at least 94%, at least 95%, at least 96%, at least 97%, at least 98%, at least 99% or more sequence identity with to amino acid sequence identified by 153-577 of SEQ ID NO:1.

The term “ID2a” as used herein refers to a domain of VAR2CSA characterized by having an amino acid sequence of at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, or at least 62, such as the 63 consecutive amino acids from the N-terminal of amino acids 578-640 of SEQ ID NO:1 and with at least 70, 75, 80, 85, 90, or 95% sequence identity to such a sequence of consecutive amino acids.

In some embodiments an amino acid sequence identity referred to herein of at least 70%, at least 80%, at least 85%, at least 90%, at least 91%, at least 92%, at least 93%, at least 94%, at least 95%, at least 96%, at least 97%, at least 98%, at least 99% or more of any one sequence identified by SEQ ID NO:1-66 or a fragment thereof, refers to a sequence with at least 71, 72, 73, 74, 75, 76, 77, 78, 79, 80, 81, 8, 83, 84, 85, 86, 87, 88, 89, 90, 91, 92, 93, 94, 95, 96, 97, 98, or 99% sequence identity to this sequence.

The terms “variant” or “variants”, as used herein, refers to a VAR2CSA polypeptide having an amino acid sequence of SEQ ID NO:55 or SEQ ID NO:56 or a fragments a VAR2CSA polypeptide comprising an amino acid sequence of SEQ ID NO:1-54, which fragments or variants retain the ability to bind chondroitin sulfate A (CSA) on proteoglycans (CSPG), wherein one or more amino acids have been substituted by another amino acid and/or wherein one or more amino acids have been deleted and/or wherein one or more amino acids have been inserted in the polypeptide and/or wherein one or more amino acids have been added to the polypeptide. Such addition can take place either at the N-terminal end or at the C-terminal end or both. The “variant” or “variants” within this definition of a VAR2CSA polypeptide still have functional activity in terms of being able to bind chondroitin sulfate A (CSA). A variant may also be a variant of another protein used according to the present invention, such as a protein of a split-protein system. In some embodiment a variant has at least 70%, such as at least 71, 72, 73, 74, 75, 76, 77, 78, 79, 80, 81, 8, 83, 84, 85, 86, 87, 88, 89, 90, 91, 92, 93, 94, 95, 96, 97, 98, or 99% sequence identity with the sequence of SEQ ID NO:1-66.

The phrases “functional variant”, “functional fragment”, and “functional derivatives” as used herein refers to variants, fragments, truncated versions, as well as derivatives of SEQ ID NO:55 or SEQ ID NO:56, which polypeptides comprises essential binding sequence parts of SEQ ID NO:55 or SEQ ID NO:56 and at least possess the ability to bind chondroitin sulfate A (CSA). It is to be understood that a VAR2CSA functional variant or functional fragment may have two or three features selected from being a both a variant, and/or a fragment and/or a derivative.

A functional variant or fragment of a VAR2CSA polypeptide encompass those that exhibit at least about 25%, such as at least about 50%, such as at least about 75%, such as at least about 90% of the binding affinity of wild-type VAR2CSA polypeptide that has been produced in the same cell type, when tested in the assays as described herein.

The term “immunologic fragment” as used herein refers to fragment of an amino acid sequence that possess essentially the same functional activities and the same spatial orientation to be recognized by an antibody. Accordingly a specific antibody will bind both the polypeptide and immunologic fragments thereof.

The term “another amino acid” as used herein means one amino acid that is different from that amino acid naturally present at that position. This includes but is not limited to amino acids that can be encoded by a polynucleotide. In some embodiments the different amino acid is in natural L-form and can be encoded by a polynucleotide.

The term “derivative” as used herein, is intended to designate a VAR2CSA polypeptide exhibiting substantially the same or improved biological activity relative to wild-type VAR2CSA identified by SEQ ID NO:55 or SEQ ID NO:56, or a fragment thereof, in which one or more of the amino acids of the parent peptide have been chemically modified, e.g. by alkylation, PEGylation, acylation, ester formation or amide formation or the like.

The term “construct” is intended to indicate a polynucleotide segment which may be based on a complete or partial naturally occurring nucleotide sequence encoding the polypeptide of interest. The construct may optionally contain other polynucleotide segments. In a similar way, the term “amino acids which can be encoded by polynucleotide constructs” covers amino acids which can be encoded by the polynucleotide constructs defined above, i.e. amino acids such as Ala, Val, Leu, Ile, Met, Phe, Trp, Pro, Gly, Ser, Thr, Cys, Tyr, Asn, Glu, Lys, Arg, His, Asp and Gln.

The term “vector”, as used herein, means any nucleic acid entity capable of the amplification in a host cell. Thus, the vector may be an autonomously replicating vector, i.e. a vector, which exists as an extra-chromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated. The choice of vector will often depend on the host cell into which it is to be introduced. Vectors include, but are not limited to plasmid vectors, phage vectors, viruses or cosmid vectors. Vectors usually contain a replication origin and at least one selectable gene, i.e., a gene which encodes a product which is readily detectable or the presence of which is essential for cell growth.

As used herein the term “appropriate growth medium” means a medium containing nutrients and other components required for the growth of cells and the expression of the nucleic acid sequence encoding the VAR2CSA polypeptide of the invention.

The term “subject” as used herein means any animal, in particular mammals, such as humans, and may, where appropriate, be used interchangeably with the term “patient”.

The term “sequence identity” as known in the art, refers to a relationship between the sequences of two or more polypeptide molecules or two or more nucleic acid molecules, as determined by comparing the sequences. In the art, “identity” also means the degree of sequence relatedness between nucleic acid molecules or between polypeptides, as the case may be, as determined by the number of matches between strings of two or more nucleotide residues or two or more amino acid residues. “Identity” measures the percent of identical matches between the smaller of two or more sequences with gap alignments (if any) addressed by a particular mathematical model or computer program (i.e., “algorithms”).

The term “similarity” is a related concept, but in contrast to “identity”, refers to a sequence relationship that includes both identical matches and conservative substitution matches. If two polypeptide sequences have, for example, (fraction ( 10/20)) identical amino acids, and the remainder are all non-conservative substitutions, then the percent identity and similarity would both be 50%. If, in the same example, there are 5 more positions where there are conservative substitutions, then the percent identity remains 50%, but the percent similarity would be 75% ((fraction ( 15/20))). Therefore, in cases where there are conservative substitutions, the degree of similarity between two polypeptides will be higher than the percent identity between those two polypeptides.

Conservative modifications to the amino acid sequence of SEQ ID NO: 1-56 (and the corresponding modifications to the encoding nucleotides) will produce VAR2CSA polypeptides having functional and chemical characteristics similar to those of naturally occurring VAR2CSA polypeptides. In contrast, substantial modifications in the functional and/or chemical characteristics of a VAR2CSA polypeptide may be accomplished by selecting substitutions in the amino acid sequence of SEQ ID NO: 1-56, and 128 that differ significantly in their effect on maintaining (a) the structure of the molecular backbone in the area of the substitution, for example, as a sheet or helical conformation, (b) the charge or hydrophobicity of the molecule at the target site, or (c) the bulk of the side chain.

For example, a “conservative amino acid substitution” may involve a substitution of a native amino acid residue with a nonnative residue such that there is little or no effect on the polarity or charge of the amino acid residue at that position. Furthermore, any native residue in the polypeptide may also be substituted with alanine, as has been previously described for “alanine scanning mutagenesis” (see, for example, MacLennan et al., 1998, Acta Physiol. Scand. Suppl. 643:55-67; Sasaki et al., 1998, Adv. Biophys. 35:1-24, which discuss alanine scanning mutagenesis).

Desired amino acid substitutions (whether conservative or non-conservative) can be determined by those skilled in the art at the time such substitutions are desired. For example, amino acid substitutions can be used to identify important residues of a polypeptide according to the present invention preferably containing VAR2CSA, or to increase or decrease the affinity of a polypeptide described herein.

Naturally occurring residues may be divided into classes based on common side chain properties:
1) hydrophobic: norleucine, Met, Ala, Val, Leu, Ile;
2) neutral hydrophilic: Cys, Ser, Thr, Asn, Gln;
3) acidic: Asp, Glu;
4) basic: His, Lys, Arg;
5) residues that influence chain orientation: Gly, Pro; and
6) aromatic: Trp, Tyr, Phe.

For example, non-conservative substitutions may involve the exchange of a member of one of these classes for a member from another class. Such substituted residues may be introduced into regions of the Plasmodium falciparum VAR2CSA polypeptide that are homologous with non-Plasmodium falciparum VAR2CSA polypeptides, or into the non-homologous regions of the molecule.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8);

phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is understood in the art. Kyte et al., J. Mol. Biol., 157:105-131 (1982). It is known that certain amino acids may be substituted for other amino acids having a similar hydropathic index or score and still retain a similar biological activity. In making changes based upon the hydropathic index, the substitution of amino acids whose hydropathic indexes are within ±2 is preferred, those that are within ±1 are particularly preferred, and those within ±0.5 are even more particularly preferred.

It is also understood in the art that the substitution of like amino acids can be made effectively on the basis of hydrophilicity, particularly where the biologically functionally equivalent protein or peptide thereby created is intended for use in immunological embodiments, as in the present case. The greatest local average hydrophilicity of a protein, as governed by the hydrophilicity of its adjacent amino acids, correlates with its immunogenicity and antigenicity, i.e., with a biological property of the protein.

The following hydrophilicity values have been assigned to amino acid residues: arginine (+3.0); lysine (′3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In making changes based upon similar hydrophilicity values, the substitution of amino acids whose hydrophilicity values are within ±2 is preferred, those that are within ±1 are particularly preferred, and those within ±0.5 are even more particularly preferred. One may also identify epitopes from primary amino acid sequences on the basis of hydrophilicity. These regions are also referred to as “epitopic core regions.”

A skilled artisan will be able to determine suitable variants of the polypeptide as set forth in SEQ ID NO:1-66 using well known techniques. For identifying suitable areas of the molecule that may be changed without destroying activity, one skilled in the art may target areas not believed to be important for activity. For example, when similar polypeptides with similar activities from the same species or from other species are known, one skilled in the art may compare the amino acid sequence of a VAR2CSA polypeptide to such similar polypeptides. With such a comparison, one can identify residues and portions of the molecules that are conserved among similar polypeptides. It will be appreciated that changes in areas of a VAR2CSA polypeptide that are not conserved relative to such similar polypeptides would be less likely to adversely affect the biological activity and/or structure of the VAR2CSA polypeptide. One skilled in the art would also know that, even in relatively conserved regions, one may substitute chemically similar amino acids for the naturally occurring residues while retaining activity (conservative amino acid residue substitutions). Therefore, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, one can predict the importance of amino acid residues in a VAR2CSA polypeptide that correspond to amino acid residues that are important for activity or structure in similar polypeptides. One skilled in the art may opt for chemically similar amino acid substitutions for such predicted important amino acid residues of VAR2CSA polypeptides and other polypeptides of the invention.

One skilled in the art can also analyze the three-dimensional structure and amino acid sequence in relation to that structure in similar polypeptides. In view of that information, one skilled in the art may predict the alignment of amino acid residues of a polypeptide with respect to its three dimensional structure. One skilled in the art may choose not to make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays as described herein. Such variants could be used to gather information about suitable variants. For example, if one discovered that a change to a particular amino acid residue resulted in destroyed, undesirably reduced, or unsuitable activity, variants with such a change would be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult J., Curr. Op. in Biotech., 7(4):422-427 (1996), Chou et al., Biochemistry, 13(2):222-245 (1974); Chou et al., Biochemistry, 113(2):211-222 (1974); Chou et al., Adv. Enzymol. Relat. Areas Mol. Biol, 47:45-148 (1978); Chou et al., Ann. Rev. Biochem., 47:251-276 and Chou et al., Biophys. J., 26:367-384 (1979). Moreover, computer programs are currently available to assist with predicting secondary structure. One method of predicting secondary structure is based upon homology modeling. For example, two polypeptides or proteins, which have a sequence identity of greater than 30%, or similarity greater than 40% often have similar structural topologies. The recent growth of the protein structural data base (PDB) has provided enhanced predictability of secondary structure, including the potential number of folds within a polypeptide's or protein's structure. See Holm et al., Nucl. Acid. Res., 27(1):244-247 (1999). It has been suggested (Brenner et al., Curr. Op. Struct. Biol., 7(3):369-376 (1997)) that there are a limited number of folds in a given polypeptide or protein and that once a critical number of structures have been resolved, structural prediction will gain dramatically in accuracy.

Additional methods of predicting secondary structure include “threading” (Jones, D., Curr. Opin. Struct. Biol., 7(3):377-87 (1997); Sippl et al., Structure, 4(1):15-9 (1996)), “profile analysis” (Bowie et al., Science, 253:164-170 (1991); Gribskov et al., Meth. Enzymol., 183:146-159 (1990); Gribskov et al., Proc. Nat. Acad. Sci., 84(13):4355-4358 (1987)), and “evolutionary linkage” (See Home, supra, and Brenner, supra).

Identity and similarity of related polypeptides can be readily calculated by known methods. Such methods include, but are not limited to, those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48:1073 (1988).

Preferred methods to determine identity and/or similarity are designed to give the largest match between the sequences tested. Methods to determine identity and similarity are described in publicly available computer programs. Preferred computer program methods to determine identity and similarity between two sequences include, but are not limited to, the GCG program package, including GAP (Devereux et al., Nucl. Acid. Res., 12:387 (1984); Genetics Computer Group, University of Wisconsin, Madison, Wis.), BLASTP, BLASTN, and FASTA (Altschul et al., J. Mol. Biol., 215:403-410 (1990)). The BLASTX program is publicly available from the National Center for Biotechnology Information (NCBI) and other sources (BLAST Manual, Altschul et al. NCB/NLM/NIH Bethesda, Md. 20894; Altschul et al., supra). The well-known Smith Waterman algorithm may also be used to determine identity.

Certain alignment schemes for aligning two amino acid sequences may result in the matching of only a short region of the two sequences, and this small aligned region may have very high sequence identity even though there is no significant relationship between the two full length sequences. Accordingly, in a preferred embodiment, the selected alignment method (GAP program) will result in an alignment that spans at least 50 contiguous amino acids of the target polypeptide.

For example, using the computer algorithm GAP (Genetics Computer Group, University of Wisconsin, Madison, Wis.), two polypeptides for which the percent sequence identity is to be determined are aligned for optimal matching of their respective amino acids (the “matched span”, as determined by the algorithm). A gap opening penalty (which is calculated as 3 times the average diagonal; the “average diagonal” is the average of the diagonal of the comparison matrix being used; the “diagonal” is the score or number assigned to each perfect amino acid match by the particular comparison matrix) and a gap extension penalty (which is usually 1/10 times the gap opening penalty), as well as a comparison matrix such as PAM 250 or BLOSUM 62 are used in conjunction with the algorithm. A standard comparison matrix (see Dayhoff et al., Atlas of Protein Sequence and Structure, vol. 5, supp.3 (1978) for the PAM 250 comparison matrix; Henikoff et al., Proc. Natl. Acad. Sci USA, 89:10915-10919 (1992) for the BLOSUM 62 comparison matrix) is also used by the algorithm.

Preferred parameters for a polypeptide sequence comparison include the following:

Algorithm: Needleman et al., J. Mol. Biol, 48:443-453 (1970); Comparison matrix: BLOSUM 62 from Henikoff et al., Proc. Natl. Acad. Sci. USA, 89:10915-10919 (1992); Gap Penalty: 12, Gap Length Penalty: 4, Threshold of Similarity: 0.

The GAP program is useful with the above parameters. The aforementioned parameters are the default parameters for polypeptide comparisons (along with no penalty for end gaps) using the GAP algorithm.

Preferred parameters for nucleic acid molecule sequence comparisons include the following: Algorithm: Needleman et al., J. Mol Biol., 48:443-453 (1970); Comparison matrix: matches=+10, mismatch=0, Gap Penalty: 50, Gap Length Penalty: 3.

The GAP program is also useful with the above parameters. The aforementioned parameters are the default parameters for nucleic acid molecule comparisons.

Other exemplary algorithms, gap opening penalties, gap extension penalties, comparison matrices, thresholds of similarity, etc. may be used, including those set forth in the Program Manual, Wisconsin Package, Version 9, September, 1997. The particular choices to be made will be apparent to those of skill in the art and will depend on the specific comparison to be made, such as DNA to DNA, protein to protein, protein to DNA; and additionally, whether the comparison is between given pairs of sequences (in which case GAP or BestFit are generally preferred) or between one sequence and a large database of sequences (in which case FASTA or BLASTA are preferred).

The inventors of the present invention has now addressed and found the answers to the following key questions related to the molecular mechanism behind placental adhesion in PM: 1) is the described differential CSA adhesion related to the VAR2CSA sequence 2) what are the exact minimal structural requirements for VAR2CSA binding to CSA 3) what type of chemical interaction exists between VAR2CSA and CSA and finally 4) can this information be used to design an optimal vaccine antigen?

By expressing identical FCR3 and 3d7 VAR2CSA truncations, the present inventors showed that VAR2CSA bind CSA with similar affinity and specificity, regardless of parasite strain origin. These two sequences has a sequence identity of 79.6%. The present inventors further demonstrate that the high CSA binding-affinity is retained in several shorter fragments, and that DBL2X, including small regions from the flanking interdomains, form a compact core that contains the high affinity CSA binding site. In silico the present inventors defined putative GAG binding sites in VAR2CSA and by deletion and substitution the present inventors showed that mutations in these sites have no effect on CSPG binding. Using the theory of polyelectrolyte-protein interactions the present inventors have shown that the VAR2CSA-CSA interaction may not, solely, be dependent on ionic interactions. Finally, the present inventors have shown that several short VAR2CSA fragments are capable of inducing the production of adhesion-blocking antibodies and that the anti-adhesive antibodies target the proposed CSA binding region. These data provide the first detailed insight into the biochemical nature of the interaction between a PfEMP1 molecule and its ligand.

Preparation of Polypeptides of the Invention

The VAR2CSA polypeptides and other polypeptides of the invention described herein may be produced by means of recombinant nucleic acid techniques and as described in WO2013/117705. In general, a cloned wild-type VAR2CSA nucleic acid sequence is modified to encode the desired protein. This modified sequence is then inserted into an expression vector, which is in turn transformed or transfected into host cells. Higher eukaryotic cells, in particular cultured mammalian cells, may be used as host cells. Procaryotic cells such as Lactococcus lactis or E. coli can also be used to express the polypeptides as long as these prokaryotes are able to produce disulfide bonds or the protein is or may be refolded correctly. In addition, Yeast strains can also be used to express the protein, here among Saccharomyces cerevisiae and P. Pichia.

The amino acid sequence alterations may be accomplished by a variety of techniques. Modification of the nucleic acid sequence may be by site-specific mutagenesis. Techniques for site-specific mutagenesis are well known in the art and are described in, for example, Zoller and Smith (DNA 3:479-488, 1984) or “Splicing by extension overlap”, Horton et al., Gene 77, 1989, pp. 61-68. Thus, using the nucleotide and amino acid sequences of VAR2CSA, one may introduce the alteration(s) of choice. Likewise, procedures for preparing a DNA construct using polymerase chain reaction using specific primers are well known to per-sons skilled in the art (cf. PCR Protocols, 1990, Academic Press, San Diego, Calif., USA).

The polypeptides of the present invention can also comprise non-naturally occurring amino acid residues. Non-naturally occurring amino acids include, without limitation, beta-alanine, desaminohistidine, trans-3-methylproline, 2,4-methanoproline, cis-4-hydroxyproline, trans-4-hydroxyproline, N-methylglycine, allo-threonine, methylthreonine, hydroxyethylcys-teine, hydroxyethylhomocysteine, nitroglutamine, homoglutamine, pipecolic acid, thiazolidine carboxylic acid, dehydroproline, 3- and 4-methylproline, 3,3-dimethylproline, tert-leucine, nor-valine, 2-azaphenylalanine, 3-azaphenylalanine, 4-azaphenylalanine, and 4-fluorophenylalanine. Several methods are known in the art for incorporating non-naturally occurring amino acid residues into polypeptides. For example, an in vitro system can be employed wherein nonsense mutations are suppressed using chemically aminoacylated suppressor tRNAs. Methods for synthesizing amino acids and aminoacylating tRNA are known in the art. Transcription and translation of plasmids containing nonsense mutations is carried out in a cell-free system comprising an E. coli S30 extract and commercially available enzymes and other reagents. Polypeptides are purified by chromatography. See, for example, Robertson et al., J. Am. Chem. Soc. 113:2722, 1991; Ellman et al., Methods Enzymol. 202:301, 1991; Chung et al., Science 259:806-9, 1993; and Chung et al., Proc. Natl. Acad. Sci. USA 90:10145-9, 1993). In a second method, translation is carried out in Xenopus oocytes by microinjection of mutated mRNA and chemically aminoacylated suppressor tRNAs (Turcatti et al., J. Biol. Chem. 271:19991-8, 1996). Within a third method, E. coli cells are cultured in the absence of a natural amino acid that is to be replaced (e.g., phenylalanine) and in the presence of the desired non-naturally occurring amino acid(s) (e.g., 2-azaphenylalanine, 3-azaphenylalanine, 4-azaphenylalanine, or 4-fluorophenylalanine). The non-naturally occurring amino acid is incorporated into the polypeptide in place of its natural counterpart. See, Koide et al., Biochem. 33:7470-6, 1994. Naturally occurring amino acid residues can be converted to non-naturally occurring species by in vitro chemical modification. Chemical modification can be combined with site-directed mutagenesis to further expand the range of substitutions (Wynn and Richards, Protein Sci. 2:395-403, 1993).

The nucleic acid construct encoding the VAR2CSA polypeptides and other polypeptides of the invention of the invention may suitably be of genomic or cDNA origin, for instance obtained by preparing a genomic or cDNA library and screening for DNA sequences coding for all or part of the polypeptide by hybridization using synthetic oligonucleotide probes in accordance with standard techniques (cf. Sambrook et al., Molecular Cloning: A Laboratory Manual, 2nd. Ed. Cold Spring Harbor Laboratory, Cold Spring Harbor, N.Y., 1989).

The nucleic acid construct encoding a VAR2CSA polypeptide may also be prepared synthetically by established standard methods, e.g. the phosphoamidite method described by Beaucage and Caruthers, Tetrahedron Letters 22 (1981), 1859-1869, or the method described by Matthes et al., EMBO Journal 3 (1984), 801-805. According to the phosphoamidite method, oligonucleotides are synthesised, e.g. in an automatic DNA synthesizer, purified, annealed, ligated and cloned in suitable vectors. The DNA sequences encoding the Plasmodium falciparum VAR2CSA polypeptides and other polypeptides of the invention may also be prepared by polymerase chain reaction using specific primers, for instance as described in U.S. Pat. No. 4,683,202, Saiki et al., Science 239 (1988), 487-491, or Sambrook et al., supra.

Furthermore, the nucleic acid construct may be of mixed synthetic and genomic, mixed synthetic and cDNA or mixed genomic and cDNA origin prepared by ligating fragments of synthetic, genomic or cDNA origin (as appropriate), the fragments corresponding to various parts of the entire nucleic acid construct, in accordance with standard techniques.

The nucleic acid construct is preferably a DNA construct. DNA sequences for use in producing VAR2CSA polypeptides and other polypeptides according to the present invention will typically encode a pre-pro polypeptide at the amino-terminus of VAR2CSA to obtain proper posttranslational processing and secretion from the host cell.

The DNA sequences encoding the Plasmodium falciparum VAR2CSA polypeptides and other polypeptides according to the present invention are usually inserted into a recombinant vector which may be any vector, which may conveniently be subjected to recombinant DNA procedures, and the choice of vector will often depend on the host cell into which it is to be introduced. Thus, the vector may be an autonomously replicating vector, i.e. a vector, which exists as an extrachromosomal entity, the replication of which is independent of chromosomal replication, e.g. a plasmid. Alternatively, the vector may be one which, when introduced into a host cell, is integrated into the host cell genome and replicated together with the chromosome(s) into which it has been integrated.

The vector is preferably an expression vector in which the DNA sequence encoding the Plasmodium falciparum VAR2CSA polypeptides and other polypeptides according to the present invention is operably linked to additional segments required for transcription of the DNA. In general, the expression vector is derived from plasmid or viral DNA, or may contain elements of both. The term, “operably linked” indicates that the segments are arranged so that they function in concert for their intended purposes, e.g. transcription initiates in a promoter and proceeds through the DNA sequence coding for the polypeptide.

Expression vectors for use in expressing VAR2CSA polypeptides and other polypeptides according to the present invention will comprise a promoter capable of directing the transcription of a cloned gene or cDNA. The promoter may be any DNA sequence, which shows transcriptional activity in the host cell of choice and may be derived from genes encoding proteins either homologous or heterologous to the host cell.

Examples of suitable promoters for directing the transcription of the DNA encoding the Plasmodium falciparum VAR2CSA polypeptide in mammalian cells are the SV40 promoter (Subramani et al., Mol. Cell Biol. 1 (1981), 854-864), the MT-1 (metallothionein gene) promoter (Palmiter et al., Science 222 (1983), 809-814), the CMV promoter (Boshart et al., Cell 41:521-530, 1985) or the adenovirus 2 major late promoter (Kaufman and Sharp, Mol. Cell. Biol, 2:1304-1319, 1982).

An example of a suitable promoter for use in insect cells is the polyhedrin promoter (U.S. Pat. No. 4,745,051; Vasuvedan et al., FEBS Lett. 311, (1992) 7-11), the P10 promoter (J. M. Vlak et al., J. Gen. Virology 69, 1988, pp. 765-776), the Autographa californica polyhedrosis virus basic protein promoter (EP 397 485), the baculovirus immediate early gene 1 promoter (U.S. Pat. Nos. 5,155,037; 5,162,222), or the baculovirus 39K delayed-early gene promoter (U.S. Pat. Nos. 5,155,037; 5,162,222).

Examples of suitable promoters for use in yeast host cells include promoters from yeast glycolytic genes (Hitzeman et al., J. Biol. Chem. 255 (1980), 12073-12080; Alber and Kawasaki, J. Mol. Appl. Gen. 1 (1982), 419-434) or alcohol dehydrogenase genes (Young et al., in Genetic Engineering of Microorganisms for Chemicals (Hollaender et al, eds.), Plenum Press, New York, 1982), or the TPI1 (U.S. Pat. No. 4,599,311) or ADH2-4c (Russell et al., Nature 304 (1983), 652-654) promoters.

Examples of suitable promoters for use in filamentous fungus host cells are, for instance, the ADH3 promoter (McKnight et al., The EMBO J. 4 (1985), 2093-2099) or the tpiA promoter. Examples of other useful promoters are those derived from the gene encoding A. oryzae TAKA amylase, Rhizomucor miehei aspartic proteinase, A. niger neutral alpha-amylase, A. niger acid stable alpha-amylase, A. niger or A. awamori glucoamylase (gluA), Rhizomucor miehei lipase, A. oryzae alkaline protease, A. oryzae triose phosphate isomerase or A. nidulans acetamidase. Preferred are the TAKA-amylase and gluA promoters. Suitable promoters are mentioned in, e.g. EP 238 023 and EP 383 779.

The DNA sequences encoding the Plasmodium falciparum VAR2CSA polypeptides and other polypeptides according to the present invention may also, if necessary, be operably connected to a suitable terminator, such as the human growth hormone terminator (Palmiter et al., Science 222, 1983, pp. 809-814) or the TPI1 (Alber and Kawasaki, J. Mol. Appl. Gen. 1, 1982, pp. 419-434) or ADH3 (McKnight et al., The EMBO J. 4, 1985, pp. 2093-2099) terminators. Expression vectors may also contain a set of RNA splice sites located downstream from the promoter and upstream from the insertion site for the VAR2CSA sequence itself. Preferred RNA splice sites may be obtained from adenovirus and/or immunoglobulin genes. Also contained in the expression vectors is a polyadenylation signal located downstream of the insertion site. Particularly preferred polyadenylation signals include the early or late polyadenylation signal from SV40 (Kaufman and Sharp, ibid.), the polyadenylation signal from the adenovirus 5 Elb region, the human growth hormone gene terminator (DeNoto et al. Nucl. Acids Res. 9:3719-3730, 1981) or the polyadenylation signal from Plasmodium falciparum, human or bovine genes. The expression vectors may also include a noncoding viral leader sequence, such as the adenovirus 2 tripartite leader, located between the promoter and the RNA splice sites; and enhancer sequences, such as the SV40 enhancer.

To direct the Plasmodium falciparum VAR2CSA polypeptides and other polypeptides of the present invention into the secretory pathway of the host cells, a secretory signal sequence (also known as a leader sequence, prepro sequence or pre sequence) may be provided in the recombinant vector. The secretory signal sequence is joined to the DNA sequences encoding the Plasmodium falciparum VAR2CSA polypeptides and other polypeptides according to the present invention in the correct reading frame. Secretory signal sequences are commonly positioned 5′ to the DNA sequence encoding the peptide. The secretory signal sequence may be that, normally associated with the protein or may be from a gene encoding another secreted protein.

For secretion from yeast cells, the secretory signal sequence may encode any signal peptide, which ensures efficient direction of the expressed Plasmodium falciparum VAR2CSA polypeptides and other polypeptides according to the present invention into the secretory pathway of the cell. The signal peptide may be naturally occurring signal peptide, or a functional part thereof, or it may be a synthetic peptide. Suitable signal peptides have been found to be the alpha-factor signal peptide (cf. U.S. Pat. No. 4,870,008), the signal peptide of mouse salivary amylase (cf. O. Hagenbuchle et al., Nature 289, 1981, pp. 643-646), a modified carboxypeptidase signal peptide (cf. L. A. Valls et al., Cell 48, 1987, pp. 887-897), the yeast BAR1 signal peptide (cf. WO 87/02670), or the yeast aspartic protease 3 (YAP3) signal peptide (cf. M. Egel-Mitani et al., Yeast 6, 1990, pp. 127-137).

For efficient secretion in yeast, a sequence encoding a leader peptide may also be inserted downstream of the signal sequence and upstream of the DNA sequence encoding the Plasmodium falciparum VAR2CSA polypeptides and other polypeptides according to the present invention. The function of the leader peptide is to allow the expressed peptide to be directed from the endoplasmic reticulum to the Golgi apparatus and further to a secretory vesicle for secretion into the culture medium (i.e. exportation of the Plasmodium falciparum VAR2CSA polypeptides and other polypeptides according to the present invention across the cell wall or at least through the cellular membrane into the periplasmic space of the yeast cell). The leader peptide may be the yeast alpha-factor leader (the use of which is described in e.g. U.S. Pat. Nos. 4,546,082, 4,870,008, EP 16 201, EP 123 294, EP 123 544 and EP 163 529). Alternatively, the leader peptide may be a synthetic leader peptide, which is to say a leader peptide not found in nature. Synthetic leader peptides may, for instance, be constructed as described in WO 89/02463 or WO 92/11378.

For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral alpha-amylase, A. niger acid-stable amylase, or A. niger glucoamylase. Suitable signal peptides are disclosed in, e.g. EP 238 023 and EP 215 594.

For use in insect cells, the signal peptide may conveniently be derived from an insect gene (cf. WO 90/05783), such as the lepidopteran Manduca sexta adipokinetic hormone precursor signal peptide (cf. U.S. Pat. No. 5,023,328).

The procedures used to ligate the DNA sequences coding for the Plasmodium falciparum VAR2CSA polypeptides and other polypeptides according to the present invention, the promoter and optionally the terminator and/or secretory signal sequence, respectively, and to insert them into suitable vectors containing the information necessary for replication, are well known to persons skilled in the art (cf., for instance, Sambrook et al., Molecular Cloning: A Laboratory Manual, Cold Spring Harbor, N.Y., 1989).

Methods of transfecting mammalian cells and expressing DNA sequences introduced in the cells are described in e.g. Kaufman and Sharp, J. Mol. Biol. 159 (1982), 601-621; Southern and Berg, J. Mol. Appl. Genet. 1 (1982), 327-341; Loyter et al., Proc. Natl. Acad. Sci. USA 79 (1982), 422-426; Wigler et al., Cell 14 (1978), 725; Corsaro and Pearson, Somatic Cell Genetics 7 (1981), 603, Graham and van der Eb, Virology 52 (1973), 456; and Neumann et al., EMBO J. 1 (1982), 841-845.

Cloned DNA sequences are introduced into cultured mammalian cells by, for example, calcium phosphate-mediated transfection (Wigler et al., Cell 14:725-732, 1978; Corsaro and Pearson, Somatic Cell Genetics 7:603-616, 1981; Graham and Van der Eb, Virology 52d:456-467, 1973) or electroporation (Neumann et al., EMBO J. 1:841-845, 1982). To identify and select cells that express the exogenous DNA, a gene that confers a selectable phenotype (a selectable marker) is generally introduced into cells along with the gene or cDNA of interest. Preferred selectable markers include genes that confer resistance to drugs such as neomycin, hygromycin, and methotrexate. The selectable marker may be an amplifiable selectable marker. A preferred amplifiable selectable marker is a dihydrofolate reductase (DHFR) sequence. Selectable markers are reviewed by Thilly (Mammalian Cell Technology, Butterworth Publishers, Stoneham, Mass., incorporated herein by reference). The person skilled in the art will easily be able to choose suitable selectable markers.

Selectable markers may be introduced into the cell on a separate plasmid at the same time as the gene of interest, or they may be introduced on the same plasmid. If on the same plasmid, the selectable marker and the gene of interest may be under the control of different promoters or the same promoter, the latter arrangement producing a dicistronic message. Constructs of this type are known in the art (for example, Levinson and Simonsen, U.S. Pat. No. 4,713,339). It may also be advantageous to add additional DNA, known as “carrier DNA,” to the mixture that is introduced into the cells.

After the cells have taken up the DNA, they are grown in an appropriate growth medium, typically 1-2 days, to begin expressing the gene of interest. As used herein the term “appropriate growth medium” means a medium containing nutrients and other components required for the growth of cells and the expression of the Plasmodium falciparum VAR2CSA polypeptide of interest. Media generally include a carbon source, a nitrogen source, essential amino acids, essential sugars, vitamins, salts, phospholipids, protein and growth factors. Drug selection is then applied to select for the growth of cells that are expressing the selectable marker in a stable fashion. For cells that have been transfected with an amplifiable selectable marker the drug concentration may be increased to select for an increased copy number of the cloned sequences, thereby in-creasing expression levels. Clones of stably transfected cells are then screened for expression of the Plasmodium falciparum VAR2CSA polypeptide of interest.

The host cell into which the DNA sequences encoding the Plasmodium falciparum VAR2CSA polypeptides and other polypeptides according to the present invention is introduced may be any cell, which is capable of producing the posttranslational modified polypeptides and includes yeast, fungi and higher eukaryotic cells.

Examples of mammalian cell lines for use in the present invention are the COS-1 (ATCC CRL 1650), baby hamster kidney (BHK) and 293 (ATCC CRL 1573; Graham et al., J. Gen. Virol. 36:59-72, 1977) cell lines. A preferred BHK cell line is the tk-ts13 BHK cell line (Waechter and Baserga, Proc. Natl. Acad. Sci. USA 79:1106-1110, 1982, incorporated herein by reference), hereinafter referred to as BHK 570 cells. The BHK 570 cell line has been deposited with the American Type Culture Collection, 12301 Parklawn Dr., Rockville, Md. 20852, under ATCC accession number CRL 10314. A tk-ts13 BHK cell line is also available from the ATCC under accession number CRL 1632. In addition, a number of other cell lines may be used within the present invention, including Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1), CHO (ATCC CCL 61) and DUKX cells (Urlaub and Chasin, Proc. Natl. Acad. Sci. USA 77:4216-4220, 1980).

Examples of suitable yeasts cells include cells of Saccharomyces spp. or Schizosaccharomyces spp., in particular strains of Saccharomyces cerevisiae or Saccharomyces kluyveri. Methods for transforming yeast cells with heterologous DNA and producing heterologous poly-peptides there from are described, e.g. in U.S. Pat. Nos. 4,599,311, 4,931,373, 4,870,008, 5,037,743, and 4,845,075, all of which are hereby incorporated by reference. Transformed cells are selected by a phenotype determined by a selectable marker, commonly drug resistance or the ability to grow in the absence of a particular nutrient, e.g. leucine. A preferred vector for use in yeast is the POT1 vector disclosed in U.S. Pat. No. 4,931,373. The DNA sequences encoding the Plasmodium falciparum VAR2CSA polypeptides and other polypeptides according to the present invention may be preceded by a signal sequence and optionally a leader sequence, e.g. as described above. Further examples of suitable yeast cells are strains of Kluyveromyces, such as K. lactis, Hansenula, e.g. H. polymorpha, or Pichia, e.g. P. pastoris (cf. Gleeson et al., J. Gen. Microbiol. 132, 1986, pp. 3459-3465; U.S. Pat. No. 4,882,279).

Examples of other fungal cells are cells of filamentous fungi, e.g. Aspergillus spp., Neurospora spp., Fusarium spp. or Trichoderma spp., in particular strains of A. oryzae, A. nidulans or A. niger. The use of Aspergillus spp. for the expression of proteins is described in, e.g., EP 272 277, EP 238 023, EP 184 438 The transformation of F. oxysporum may, for instance, be carried out as described by Malardier et al., 1989, Gene 78: 147-156. The transformation of Trichoderma spp. may be performed for instance as described in EP 244 234.

When a filamentous fungus is used as the host cell, it may be transformed with the DNA construct of the invention, conveniently by integrating the DNA construct in the host chromosome to obtain a recombinant host cell. This integration is generally considered to be an advantage as the DNA sequence is more likely to be stably maintained in the cell. Integration of the DNA constructs into the host chromosome may be performed according to conventional methods, e.g. by homologous or heterologous recombination.

Transformation of insect cells and production of heterologous polypeptides therein may be performed as described in U.S. Pat. Nos. 4,745,051; 4,879,236; 5,155,037; 5,162,222; EP 397,485) all of which are incorporated herein by reference. The insect cell line used as the host may suitably be a Lepidoptera cell line, such as Spodoptera frugiperda cells or Trichoplusia ni cells (cf. U.S. Pat. No. 5,077,214). Culture conditions may suitably be as described in, for instance, WO 89/01029 or WO 89/01028, or any of the aforementioned references.

The transformed or transfected host cell described above is then cultured in a suitable nutrient medium under conditions permitting expression of the Plasmodium falciparum VAR2CSA polypeptide after which all or part of the resulting peptide may be recovered from the culture. The medium used to culture the cells may be any conventional medium suitable for growing the host cells, such as minimal or complex media containing appropriate supplements. Suitable media are available from commercial suppliers or may be prepared according to published recipes (e.g. in catalogues of the American Type Culture Collection).

The Plasmodium falciparum VAR2CSA polypeptide produced by the cells may then be recovered from the culture medium by conventional procedures including separating the host cells from the medium by centrifugation or filtration, precipitating the proteinaqueous components of the supernatant or filtrate by means of a salt, e.g. ammonium sulfate, purification by a variety of chromatographic procedures, e.g. ion exchange chromatography, gelfiltration chromatography, affinity chromatography, or the like, dependent on the type of polypeptide in question.

Transgenic animal technology may be employed to produce the VAR2CSA polypeptides and other polypeptides of the invention. It is preferred to produce the proteins within the mammary glands of a host female mammal. Expression in the mammary gland and subsequent secretion of the protein of interest into the milk overcomes many difficulties encountered in isolating proteins from other sources. Milk is readily collected, available in large quantities, and biochemically well characterized. Furthermore, the major milk proteins are present in milk at high concentrations (typically from about 1 to 15 g/l).

From a commercial point of view, it is clearly preferable to use as the host a species that has a large milk yield. While smaller animals such as mice and rats can be used (and are preferred at the proof of principle stage), it is preferred to use livestock mammals including, but not limited to, pigs, goats, sheep and cattle. Sheep are particularly preferred due to such factors as the previous history of transgenesis in this species, milk yield, cost and the ready availability of equipment for collecting sheep milk (see, for example, WO 88/00239 for a comparison of factors influencing the choice of host species). It is generally desirable to select a breed of host animal that has been bred for dairy use, such as East Friesland sheep, or to introduce dairy stock by breeding of the transgenic line at a later date. In any event, animals of known, good health status should be used.

To obtain expression in the mammary gland, a transcription promoter from a milk protein gene is used. Milk protein genes include those genes encoding caseins (see U.S. Pat. No. 5,304,489), beta lactoglobulin, a lactalbumin, and whey acidic protein. The beta lactoglobulin (BLG) promoter is preferred. In the case of the ovine beta lactoglobulin gene, a region of at least the proximal 406 bp of 5′ flanking sequence of the gene will generally be used, although larger portions of the 5′ flanking sequence, up to about 5 kbp, are preferred, such as a ˜4.25 kbp DNA segment encompassing the 5′ flanking promoter and non-coding portion of the beta lactoglobulin gene (see Whitelaw et al., Biochem. J. 286: 31 39 (1992)). Similar fragments of promoter DNA from other species are also suitable.

Other regions of the beta lactoglobulin gene may also be incorporated in constructs, as may genomic regions of the gene to be expressed. It is generally accepted in the art that constructs lacking introns, for example, express poorly in comparison with those that contain such DNA sequences (see Brinster et al., Proc. Natl. Acad. Sci. USA 85: 836 840 (1988); Palmiter et al., Proc. Natl. Acad. Sci. USA 88: 478 482 (1991); Whitelaw et al., Transgenic Res. 1: 3 13 (1991); WO 89/01343; and WO 91/02318, each of which is incorporated herein by reference). In this regard, it is generally preferred, where possible, to use genomic sequences containing all or some of the native introns of a gene encoding the protein or polypeptide of interest, thus the further inclusion of at least some introns from, e.g, the beta lactoglobulin gene, is preferred. One such region is a DNA segment that provides for intron splicing and RNA polyadenylation from the 3′ non-coding region of the ovine beta lactoglobulin gene. When substituted for the natural 3′ non-coding sequences of a gene, this ovine beta lactoglobulin segment can both enhance and stabilize expression levels of the protein or polypeptide of interest. Within other embodiments, the region surrounding the initiation ATG of the VAR2CSA sequence is replaced with corresponding sequences from a milk specific protein gene. Such replacement provides a putative tissue specific initiation environment to enhance expression. It is convenient to replace the entire VAR2CSA pre pro and 5′ non-coding sequences with those of, for example, the BLG gene, although smaller regions may be replaced.

For expression of VAR2CSA polypeptides and other polypeptides according to the present invention in transgenic animals, a DNA segment encoding VAR2CSA is operably linked to additional DNA segments required for its expression to produce expression units. Such additional segments include the above mentioned promoter, as well as sequences that provide for termination of transcription and polyadenylation of mRNA. The expression units will further include a DNA segment encoding a secretory signal sequence operably linked to the segment encoding modified VAR2CSA. The secretory signal sequence may be a native secretory signal sequence or may be that of another protein, such as a milk protein (see, for example, von Heijne, Nucl. Acids Res. 14: 4683 4690 (1986); and Meade et al., U.S. Pat. No. 4,873,316, which are incorporated herein by reference).

Construction of expression units for use in transgenic animals is carried out by inserting a VAR2CSA sequence into a plasmid or phage vector containing the additional DNA segments, although the expression unit may be constructed by essentially any sequence of ligations. It is particularly convenient to provide a vector containing a DNA segment encoding a milk protein and to replace the coding sequence for the milk protein with that of a VAR2CSA variant; thereby creating a gene fusion that includes the expression control sequences of the milk protein gene. In any event, cloning of the expression units in plasmids or other vectors facilitates the amplification of the VAR2CSA sequence. Amplification is conveniently carried out in bacterial (e.g. E. coli) host cells, thus the vectors will typically include an origin of replication and a selectable marker functional in bacterial host cells. The expression unit is then introduced into fertilized eggs (including early stage embryos) of the chosen host species. Introduction of heterologous DNA can be accomplished by one of several routes, including microinjection (e.g. U.S. Pat. No. 4,873,191), retroviral infection (Jaenisch, Science 240: 1468 1474 (1988)) or site directed integration using embryonic stem (ES) cells (reviewed by Bradley et al., Bio/Technology 10: 534 539 (1992)). The eggs are then implanted into the oviducts or uteri of pseudopregnant females and allowed to develop to term. Offspring carrying the introduced DNA in their germ line can pass the DNA on to their progeny in the normal, Mendelian fashion, allowing the development of transgenic herds. General procedures for producing transgenic animals are known in the art (see, for example, Hogan et al., Manipulating the Mouse Embryo: A Laboratory Manual, Cold Spring Harbor Laboratory, 1986; Simons et al., Bio/Technology 6: 179 183 (1988); Wall et al., Biol. Reprod. 32: 645 651 (1985); Buhler et al., Bio/Technology 8: 140 143 (1990); Ebert et al., Bio/Technology 9: 835 838 (1991); Krimpenfort et al., Bio/Technology 9: 844 847 (1991); Wall et al., J. Cell. Biochem. 49: 113 120 (1992); U.S. Pat. No. 4,873,191; 4,873,316; WO 88/00239, WO 90/05188, WO 92/11757; and GB 87/00458). Techniques for introducing foreign DNA sequences into mammals and their germ cells were originally developed in the mouse (see, e.g., Gordon et al., Proc. Natl. Acad. Sci. USA 77: 7380 7384 (1980); Gordon and Ruddle, Science 214: 1244 1246 (1981); Palmiter and Brinster, Cell 41: 343 345 (1985); Brinster et al., Proc. Natl. Acad. Sci. USA 82: 4438 4442 (1985); and Hogan et al. (ibid.)). These techniques were subsequently adapted for use with larger animals, including livestock species (see, e.g., WO 88/00239, WO 90/05188, and WO 92/11757; and Simons et al., Bio/Technology 6: 179 183 (1988)). To summarize, in the most efficient route used to date in the generation of transgenic mice or livestock, several hundred linear molecules of the DNA of interest are injected into one of the pro nuclei of a fertilized egg according to established techniques. Injection of DNA into the cytoplasm of a zygote can also be employed.

Production in transgenic plants may also be employed. Expression may be generalised or directed to a particular organ, such as a tuber (see, Hiatt, Nature 344:469 479 (1990); Edelbaum et al., J. Interferon Res. 12:449 453 (1992); Sijmons et al., Bio/Technology 8:217 221 (1990); and EP 0 255 378).

The Split-Protein Binding System (SPBS):

In the present invention, a split-protein binding system, such as the SpyTag-SpyCatcher system may be used to attach a VAR2CSA polypeptide to a diagnostic or detection moiety. However any split protein system can be used, for example the Sdy/DANG catcher system described in (Tan L L, Hoon S S, Wong F T (2016) Kinetic Controlled Tag-Catcher Interactions for Directed Covalent Protein Assembly. PLoS ONE 11(10): e0165074. https://doi.org/10.1371/journal.pone.0165074) (SEQ ID NO:66).The interaction between SpyTag and SpyCatcher occurs when the un-protonated amine of Lys31 nucleophilically attacks the carbonyl carbon of Asp117, catalyzed by the neighboring Glu77. The minimal peptide to mediate this binding is AHIVMVDA (SEQ ID NO:59) whereas a c-terminal extension giving the sequence: AHIVMVDAYKPTK (SEQ ID NO: 58) provides the most optimal region, designated “SpyTag” (Zakeri et al PNAS 2012). A recombinantly expressed VAR2CSA polypeptide may be designed to include this 13 amino acids peptide (SpyTag) N-terminally, which enables covalent isopeptide bond formation to a biotinylated 12 kDa SpyCatcher protein, which in turn may be attached to biotin binding moieties, such as CELLection™ Biotin Binder Dynabeads®.

SpyCatcher is a part of the CnaB2 domain from the FbaB protein from Streptococcus pyogenes and binds SpyTag consisting of another part of the CnaB2 domain. When these two polypeptides of the CnaB2 domain are mixed, they will spontaneously form an irreversible isopeptide bond thereby completing the formation of the CnaB2 domain.

A K-Tag/SpyTag/SpyLigase system may also be used in the present invention. The CnaB2 domain from Streptococcus pyogenes can be used to generate a covalent peptide-peptide ligation system (Fierer J O. et al. 2014). This is done by splitting the CnaB2 into three parts a) the 13 amino acid SpyTag (SEQ ID NO: 58), b) the p-strand of CnaB2 (SEQ ID NO 60)) named K-Tag, and c) the SpyLigase (SEQ ID NO: 61) constructed from the remaining SpyCatcher polypeptide. By expressing a VAR2CSA polypeptide with the small K-Tag fused at the C- or N-terminus and mixing that fusion protein with SpyTag-displaying diagnostic moieties, such as biotinylated SpyTag fragment together with the SpyLigase, the K-Tag-fusion antigen will be attached to the SpyTag-biotin by covalent ligation of the SpyTag with the K-Tag facilitated by the SpyLigase. Conversely, the K-Tag may also be inserted on a diagnostic moiety, such as biotinylated K-Tag whereby the VAR2CSA polypeptide should then be fused to the SpyTag at the C- or N-terminus.

As part of a similar strategy for covalent coupling of a VAR2CSA polypeptide to a diagnostic moiety, another pair of split-protein binding partners may be used in the present invention. The major pilin protein, Spy0128, from Streptococcus pyogenes can be split into two fragments (split-Spy0128 (residues 18-299 of Spy0128) (SEQ ID NO 63) and isopeptide (residues 293-308 of Spy0128 (TDKDMTITFTNKKDAE))) (SEQ ID NO 62), which together are capable of forming an intermolecular covalent complex (Zakeri, B. et al. 2010). In line with the described SpyTag-SpyCatcher strategy, the Spy0128 isopeptide can be inserted into the either the detection moiety or the VAR2CSA polypeptide and the same for the split-Spy0128 binding partner. Again simple mixing, or pre-injection of tagged VAR2CSA polypeptide and detection moiety will result in a covalent interaction between the two. It is to be understood that the SpyTag may be replaced by a Spy0128 isopeptide, and the SpyCatcher replaced by split-Spy0128.

Specific embodiments of the invention:

As described above one aspect of the present invention relates to method for the identification of a trophoblast and/or fetal cell in a biological sample, the method comprising:

a) contacting a biological sample comprising trophoblast and/or fetal cells expressing CSA with a VAR2CSA polypeptide, or a conjugate or fusion protein thereof; and

b) detecting said VAR2CSA polypeptide or conjugate or fusion protein thereof specifically bound to said trophoblast and/or fetal cells expressing CSA.

In some embodiments the method further comprises a step c) of isolating from the biological sample said trophoblast and/or fetal cells expressing CSA specifically bound to said VAR2CSA polypeptide or conjugate or fusion protein thereof.

In some embodiments the method further comprises a previous step of obtaining a biological sample comprising trophoblast and/or fetal cells expressing CSA from a subject, such as a pregnant female subject, such as a human female subject.

In some embodiments the biological sample is or comprises peripheral blood.

In some embodiments the biological sample is derived from a pregnant female subject, such as a human female subject.

In some embodiments the method detects a circulating trophoblast and/or fetal cell in the peripheral blood of a pregnant female, such as a human female subject.

In some embodiments the VAR2CSA polypeptide, or a conjugate or fusion protein thereof comprises a detectable label or diagnostic effector moiety, such as a fluorescent or radioactive label, and/or a carrier for detection, such as a magnetic bead. A “diagnostic effector moiety” may be any atom, molecule, or compound that is useful in diagnosing a disease. Useful diagnostic agents include, but are not limited to, radioisotopes, dyes, contrast agents, fluorescent compounds or molecules, enhancing agents (e.g., paramagnetic ions), or beads or other conjugates for collection. It is to be understood that a magnetic bead used according to the present invention may be any suitable magnetic bead used for standard purification or separation. Accordingly a magnetic bead may be ferromagnetic or paramagnetic or superparamagnetic, such as permanent magnets or materials attracted to magnetic materials.

In some embodiments the VAR2CSA polypeptide consist of or comprises SEQ ID NO:55 or SEQ ID NO:56 or fragments or variants thereof with the ability to bind chondroitin sulfate A (CSA) that could be presented on a proteoglycans (CSPG).

In some embodiments the VAR2CSA polypeptide is a fragment of VAR2CSA that consist of a sequential amino acid sequence of

a. ID1, and

b. DBL2Xb, and optionally

c. ID2a.

In some embodiments the VAR2CSA polypeptide binds chondroitin sulfate A (CSA) on proteoglycans (CSPG) with an affinity as measured by a KD lower than 100 nM, such as lower than 80 nM, such as lower than 70 nM, such as lower than 60 nM, such as lower than 50 nM, such as lower than 40 nM, such as lower than 30 nM, such as lower than 26 nM, such as lower than 24 nM, such as lower than 22 nM, such as lower than 20 nM, such as lower than 18 nM, such as lower than 16 nM, such as lower than 14 nM, such as lower than 12 nM, such as lower than 10 nM, such as lower than 9 nM, such as lower than 8 nM, such as lower than 7 nM, such as lower than 6 nM, or lower than 4nM.

In some embodiments the VAR2CSA polypeptide comprises or consist of an amino acid sequence having at least 70, 75, 80, 85, 90, or 95% sequence identity with any one amino acid sequence of 1-577 of SEQ ID NO:1, 1-640 of SEQ ID NO:1, 65-640 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54.

In some embodiments the VAR2CSA polypeptide comprises or consist of an amino acid sequence having at least 70, 75, 80, 85, 90, or 95% sequence identity with an amino acid sequence of 578-640 of SEQ ID NO:1, 593-656 of SEQ ID NO:3, 580-643 of SEQ ID NO:4, 577-640 of SEQ ID NO:5, 587-650 of SEQ ID NO:10, 580-643 of SEQ ID NO:11, 566-628 of SEQ ID NO:29, 585-647 of SEQ ID NO:34, 570-632 of SEQ ID NO:36, 576-639 of SEQ ID NO:37, 593-655 of SEQ ID NO:38, 604-667 of SEQ ID NO:41, 589-652 of SEQ ID NO:43, 566-628 of SEQ ID NO:44, 590-653 of SEQ ID NO:45, 574-637 of SEQ ID NO:48, 584-646 of SEQ ID NO:53, or 570-632 of SEQ ID NO:54.

In some embodiments the VAR2CSA polypeptide comprises or consist of an amino acid sequence having at least 70, 75, 80, 85, 90, or 95% sequence identity with an amino acid sequence of SEQ ID NO:1, 2, 6, 8, 9, 12, 13, 14, 15, 16, 17, 18, 19, 20, 21, 22, 23, 24, 25, 26, 27, 28, 30, 31, 32, 33, 35, 39, 40, 42, 46, 47, 49, 50, 51, 52.

In some embodiments the VAR2CSA polypeptide consists of an amino acid sequence having at least 70, 75, 80, 85, 90, or 95% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54.

In some embodiments the VAR2CSA polypeptide consists of an amino acid sequence selected from the list consisting of SEQ ID NO:1, 3-5, 10, 11, 29, 34, 36-38, 41, 43-45, 48, 53, 54.

In some embodiments the VAR2CSA polypeptide consists of an amino acid sequence having a length of less than 700 amino acids, such as less than 690 amino acids, such as less than 680 amino acids, such as less than 670 amino acids, such as less than 660 amino acids, such as less than 650 amino acids, such as less than 640 amino acids, such as less than 630 amino acids, such as less than 620 amino acids, such as less than 610 amino acids, such as less than 600 amino acids, such as less than 590 amino acids, such as less than 580 amino acids, such as less than 570 amino acids.

In some embodiments the VAR2CSA polypeptide or a conjugate or fusion protein thereof comprises a peptide part of a split-protein binding system. In some embodiments the peptide part of a split-protein binding system is selected from K-Tag (SEQ ID NO:60), SpyCatcher (SEQ ID NO:57), SpyCatcher-AN (SEQ ID NO:64), SpyTag (SEQ ID NO:58), Minimal Spytag sequence (SEQ ID NO:59), split-Spy0128 (SEQ ID NO:63), isopeptide Spy0128 (SEQ ID NO:62), or the peptide part of the Sdy/DANG catcher system (SEQ ID NO:66) or any inverse sequence thereof, or a variant thereof with sequence identity of at least about 80%, such as at least about 82, 84, 86, 88, 90, 92, 94, 96, 98, or 99%.

In some embodiments the VAR2CSA polypeptide or a conjugate or fusion protein thereof is a conjugate with a magnetic bead.

Sequences, including sequences of VAR2CSA polypeptides:

>fcr3 745 amino acids | 640 aa; underlined sequence
corresponds to the ID1 domain of FCR3, Sequence in
bold corresponds to DBL2Xb domain of FCR3. Remaining
sequence is ID2a
(SEQ ID NO: 1)
NYIKGDPYFAEYATKLSFILNPSDANNPSGETANHNDEACNCNESGISSVGQAQTSGPSSNKTCITHSSIK
TNKKKECKDVKLGVRENDKDLKICVIEDTSLSGVDNCCCQDLLGILQENCSDNKRGSSSNDSCDNKNQDEC
QKKLEKVFASLTNGYKCDKCKSGTSRSKKKWIWKKSSGNEEGLQEEYANTIGLPPRTQSLYLGNLPKLENV
CEDVKDINFDTKEKFLAGCLIVSFHEGKNLKKRYPQNKNSGNKENLCKALEYSFADYGDLIKGTSIWDNEY
TKDLELNLQNNFGKLFGKYIKKNNTAEQDTSYSSLDELRESWWNTNKKYIWTAMKHGAEMNITTCNADGSV
TGSGSSCDDIPTIDLIPQYLRFLQEWVENFCEQRQAKVKDVITNCKSCKESGNKCKTECKTKCKDECEKYK
KFIEACGTAGGGIGTAGSPWSKRWDQIYKRYSKHIEDAKRNRKAGTKNCGTSSTTNAAASTDENKCVQSDI
DSFFKHLIDIGLTTPSSYLSNVLDDNICGADKAPWTTYTTYTTTEKCNKERDKSKSQSSDTLVVVNVPSPL
GNTPYRYKYACQCKIPTNEETCDDRKEYMNQWSCGSARTMKRGYKNDNYELCKYNGVDVKPTTVRSNSSKL
D
>gi|254952610|gb|ACT97135.1| VAR2CSA [Plasmodium falciparum] |
341 aa
(SEQ ID NO: 2)
KCDKCKSGTSRSRKIWTWRKSSGNKEGLQEEYANTIGLSPRTQLLYLGNLRKLENVCEDVTDINFDTKEKF
LAGCLIAAFHEGKNLKKRYLEKKKGDNNSKLCKDLKYSFADYGDLIKGTSIWDNDFTKDLELNLQQIFGKL
FRKYIKKKNISTEQDTSYSSLDELRESWWNTNKKYIWLAMKHGAGMNSTTCSCSGDSSSGENQTNSCDDIP
TIDLIPQYLRFLQEWVEHFCEQRQAKVKDVITNCNSCKESGGTCNSDCEKKCKNKCDAYKTFIEDCKGVGG
TGTAGSSWVKRWYQIYMRYSKYIEDAKRNRKAGTKSCGTSSTTNVSVSTDENKCVQS-
>M24 745 amino acids | 656 aa
(SEQ ID NO: 3)
DYIKGDPYFAEYATKLSFILNSSDANNPSGETANHNDEVCNPNESEISSVGQAQTSDPSSNKTCNTHSSIK
ANKKKVCKHVKLGINNNDKVLRVCVIEDTSLSGVENCCFKDLLGILQENCSDNKSGSSSNGSCNNKNQEAC
EKNLEKVLASLTNCYKCDKCKSGTSTVNKNWIWKKSSGNKEGLQKEYANTIGLPPRTHSLYLGNLPKLENV
CEDVKDINFDTKEKFLAGCLIAAFHEGKNLKKRYPQNKNDDNNSKLCKALEYSFADYGDLIKGTSIWDNEY
TKDLELNLQQIFGKLFRKYIKKNISTEQDTLYSSLDELRESWWNTNKKYIWLAMKHGAGMNITTCCGDGSV
TGSGSSCDDIPTIDLIPQYLRFLQEWVEHFCKQRQEKVKDVINSCNSCKNTSSKTKLGDTCNSDCEKKCKI
ECEKYKKFIEECRTAVGGTAGSSWSKRWDQIYKMYSKHIEDAKRNRKAGTKNCGITTGTISGESSGANSGV
TTTENKCVQSDIDSFFKHLIDIGLTTPSSYLSIVLDDNICGDDKAPWTTYTTYTTYTTTEKCNKERDKSKS
QQSNTSVVVNVPSPLGNTPHGYKYACQCKIPTNEETCDDRKEYMNQWISDTSKNPKGSGSTNNDYELYTYN
GVKETKLPKKLNSPKLD
>KMWII 745 amino acids | 643 aa
(SEQ ID NO: 4)
DYIKDDPYSKEYTTKLSFILNSSDANTSSGETANHNDEACNCNESEISSVGQAQTSGPSSNKTCITHSFIK
ANKKKVCKDVKLGVRENDKVLRVCVIEDTSLSGVDNCCCQDLLGILQENCSDNKRGSSSNGSCNNKNQDEC
QKKLEKVFVSLTNGYKCDKCKSGTSTVNKKWIWKKSSGNEKGLQKEYANTIGLPPRTQSLYLGNLPKLGNV
CEDVTDINFDTKEKFLAGCLIAAFHEGKNLKISHEKKKGDNGKKLCKALEYSFADYGDLIKGTSIWDNEYT
KDLELNLQKAFGKLFGKYIKKNIASDENTSYSSLDELRESWWNTNKKYIWTAMKHGAEMNSTMCNADGSVT
GSGSSCDDIPTTDFIPQYLRFLQEWVEHFCKQRQEKVNAVIENCNSCKNTSGERKIGGTCNGDCKTECKNK
CEAYKNFIEDCKGGDGTAGSSWVKRWDQIYKRYSKHIEDAKRNRKAGTKSCGPSSITNASVSTDENKCVQS
DIDSFFKHLIDIGLTTPSSYLSIVLDENNCGEDNAPWTTYTTYTTTEKCNKDKKKSKSQSCNTAVVVNVPS
PLGNTPHEYKYACQCKIPTTEETCDDRKEYMNQWISDTSKKQKGSGSTNNDYELYTYTGVKETKLPKKLNS
PKLD
>1248 745 amino acids | 640 aa
(SEQ ID NO: 5)
SYVKNDPYSKEYVTKLSFILNPSDANNPSGETANHNDEACNPNESEIASVGQAQTSDRLSQKACITHSFIG
ANKKIVCKDVKLGVREKDKDLKICVIEDDSLRGVENCCFKDLLGILQENCSDNKSGSSSNGSCNNKNQDEC
QKKLDEALASLHNGYKCDKCKSGTSRSKKIWTWRKFPGNGEGLQKEYANTIGLPPRTQSLYLGNLRKLENV
CKGVTDINFDTKEKFLAGCLIAAFHEGKNLKISNKKKNDDNGKKLCKDLKYSFADYGDLIKGTSIWDNEYT
KDLELNLQKIFGKLFRKYIKKNIASDENTLYSSLDELRESWWNTNKKYIWLAMKHGTTCSSGSGDNGDGSV
TGSGSSCDDMSTIDLIPQYLRFLQEWVEHFCKQRQEKVKDVIENCKSCKNTSGERIIGGTCGSDCKTKCKG
ECDAYKNFIEECKRGDGTAGSPWSKRWDQIYMRYSKYIEDAKRNRKAGTKNCGTSSTTNAAENKCVQSDID
SFFKHLIDIGLTTPSSYLSIVLDENICGDDKAPWTTYTTYTTTEKCNKETDKSKSQSCNTAVVVNVPSPLG
NTPHGYKYACECKIPTTEETCDDRKEYMNQWISDTSKKPKGGRSTNNDYELYTYNGVKETKLPKKSSSSKL
D
>gi|254952618|gb|ACT97139.1| VAR2CSA [Plasmodium falciparum] |
358 aa
(SEQ ID NO: 6)
KCEKCKSEQSKKNNNIWIWRKFPGNGEGLQKEYANTIGLPPRTHSLYLGNLPKLENVCKDVKDINFDTKEK
FLAGCLIAAFHEGKNLKTTYPQNKNADNNSKLCKDLKYSFADYGDLIKGTSIWDNDFTKDLELNLQKIFGK
LFRKYIKKNIASDENTLYSSLDELRESWWNTNKKYIWLAMKHGAEMNSTMCNGDGSVTGSSDSGSTTCSGD
NGSISCDDIPTIDLIPQYLRFLQEWVEHFCKQRQEKVKPVIENCKSCKNTSGERIIGGTCGSDCEKKCKGE
CDAYKKFIEECKGGGGGTGTAGSPWSKRWDQIYKRYSKYIEDAKRNRKAGTKSCGPSSTTNAAASTTESKC
VQS
>gi|254952592|gb|ACT97126.1| VAR2CSA [Plasmodium falciparum] |
333 aa
(SEQ ID NO: 7)
KCDKCKSEQSKKNNKNWIWKQFPGNGEGLQKEYANTIGLPPRTHSLYLGNLPKLENVCKGVTDINFDTKEK
FLAGCLIAAFHEGKNLKTSHEKKKGDNGKKLCKDLKYSFADYGDLIKGTSIWDNDFTKDLELNLQQIFGKL
FRKYIKKNISAEQDTSYSSLDELRESWWNTNKKYIWLAMKHGTTCSSGSGDNGDGSVTGSGSSCDDMPTTD
FIPQYLRFLQEWVEHFCKQRQEKVNAVITNCKSCKESGGTCNSDCEKKCKDECEKYKKFIEECRTAADGTA
GSSWSKRWDQIYKMYSKHIEDAKRNRKAGTKNCGTSSTTNAAENKCVQS
>gi|90193467|gb|ABD92329.1| erythrocyte membrane protein 1
[Plasmodium falciparum] | 269 aa
(SEQ ID NO: 8)
DYIKDDPYSKEYTTKLSFILNSSDANTSSGETANHNDEACNCNESEIASVEQASISDRSSQKAYITHSSIK
TNKKKVCKYVKLGINNNDKVLRVCVIEDTSLSGVENCCFKDLLGILQENCSDNKRGSSFNDSCNNNNEEAC
QKKLEKVLASLTNGYKCEKCKSGTSRSKKKWIWKKSSGKEGGLQKEYANTIGLPPRTQSLYLGNLPKLENV
CKGVTDINFDTKEKFLAGCLIAAFHEGKNLKPSHQNKNDDNNSKLCKDLKYSFADY
>gi|254952616|gb|ACT97138.1| VAR2CSA [Plasmodium falciparum] |
333 aa
(SEQ ID NO: 9)
KCDKCKSGTSRSKKKWTWRKSSGNKEGLQKEYANTIGLPPRTHSLYLGNLRKLENVCEDVTDINFDTKEKF
LAGCLIAAFHEGKNLKTTYPQNKNDDNNSKLCKALKYSFADYGDLIKGTSIWDNDFTKDLELNLQKIFGKL
FRKYIKKNISTEQHTSYSSLDELRESWWNTNKKYIWLAMKHGAEMNGTTCSCSGDSSDDIPTIDLIPQYLR
FLQEWVEHFCKQRQAKVNAVINSCNSCKNTSGERKLGGTCGSECKTECKNKCDAYKEFIDGTGSGGGTGTA
GSSWVKRWDQIYKRYSKYIEDAKRNRKAGSKNCGTSSTTNAAESKCVQS
>hb31 745 amino acids | 650 aa
(SEQ ID NO: 10)
SYVKNNPYSAEYVTKLSFILNSSDANTSSETPSKYYDEVCNCNESEISSVGQAQTSGPSSNKTCITHSSIK
TNKKKVCKDVKLGINNNDKVLRVCVIEDTSLSGVDNCCCQDLLGILQENCSDKNQSGSSSNGSCNNKNQDE
CQKKLEKVFASLTNGYKCDKCKSGTSRSKKKWIWRKSSGNEEGLQKEYANTIGLPPRTQSLYLGNLRKLEN
VCKGVTDINFDTKEKFLAGCLIAAFHEGKNLKTTYPQNKKKLCKDLKYSFADYGDLIKGTSIWDNEYTKDL
ELNLQKAFGKLFRKYIKKNISTEQHTLYSSLDELRESWWNTNKKYIWLAMKHGAGMNSTTCCGDGSVTGSG
SSCDDIPTIDLIPQYLRFLQEWVEHFCKQRQEKVNAVIENCNSCKECGDTCNGECKTECEKKCKIECEKYK
TFIEECVTAVGGTSGSPWSKRWDQIYKRYSKYIEDAKRNRKAGTKNCGITTGTISGESSGANSGVTTTENK
CVQSDIDSFFKHLIDIGLTTPSSYLSIVLDDNICGADNAPWTTYTTYTTYTTTKNCDIKKKTPKSQPINTS
VVVNVPSPLGNTPHGYKYACQCKIPTTEESCDDRKEYMNQWIIDTSKKQKGSGSTNNDYELYTYNGVKETK
LPKKSSSSKLD
>hb32 745 amino acids | 643 aa
(SEQ ID NO: 11)
SYVKDDPYSAEYVTKLSFILNSSDANTSSETPSKYYDEVCNCNESEISSVGQAQTSGPSSNKTCITHSSIK
TNKKKVCKDVKLGINNNDKVLRVCVIEDTSLSGVDNCCCQDLLGILQENCSDKNQSGSSSNGSCNNKNQDE
CQKKLEKVFASLTNGYKCDKCKSGTSRSKKKWIWRKSSGNEEGLQKEYANTIGLPPRTQSLYLGNLPKLEN
VCKGVTDIIYDTKEKFLSGCLIAAFHEGKNLKTSHEKKNDDNGKKLCKALEYSFADYGDLIKGTSIWDNDF
TKDLELNLQKIFGKLFRKYIKKNNTAEQDTSYSSLDELRESWWNTNKKYIWTAMKHGAGMNSTTCSGDGSV
TGSGSSCDDMPTIDLIPQYLRFLQEWVEHFCKQRQEKVKDVITNCNSCKECGDTCNGECKTECKTKCKGEC
EKYKNFIEECNGTADGGTSGSSWSKRWDQIYKRYSKYIEDAKRNRKAGTKNCGTSSTTNAAASTTENKCVQ
SDIDSFFKHLIDIGLTTPSSYLSNVLDDNICGEDKAPWTTYTTYTTKNCDIQKKTPKPQSCDTLVVVNVPS
PLGNTPHGYKYVCECKIPTTEETCDDRKEYMNQWIIDTSKKQKGSGSTNNDYELYTYNGVQIKQAAGTLKN
SKLD
>gi|90193475|gb|ABD92333.1| erythrocyte membrane protein 1
[Plasmodium falciparum] | 269 aa
(SEQ ID NO: 12)
NYIKGDPYSAEYATKLSFILNSSDTENASEKIQKNNDEVCNCNESEIASVEQAPISDRSSQKACITHSSIK
ANKKKVCKHVKLGVRENDKDLKICVIEDTSLSGVDNCCCQDLLGILQENCSDNKSGSSSNGSCNNNNEEIC
QKKLEKVLASLTNGYKCDKCKSGTSTVNKNWIWKKYSGKEGGLQEEYANTIGLPPRTQSLYLGNLPKLENV
CEDVKDINFDTKEKFLAGCLIAAFHEGKNLKTSNKKKNDDNNSKLCKALKYSFADY
>gi|254952600|gb|ACT97130.1| VAR2CSA [Plasmodium falciparum] |
344 aa
(SEQ ID NO: 13)
KCDKCKSGTSTVNKKWIWKKYSGTEGGLQEEYANTIALPPRTQSLYLGNLPKLENVCKDVTDINFDTKEKF
LAGCLIAAFHEGKNLKTTYLEKKKGDNGKKNDDNNSKLCKALKYSFADYGDLIKGTSIWDNDFTKDLELNL
QQIFGKLFRKYIKKNIASDENTLYSSLDELRESWWNTNKKYIWLAMKHGAGMNSTMCNADGSVTGSGSSCD
DIPTIDLIPQYLRFLQEWVEHFCKQRQAKVKDVITNCNSCKECGGTCNGECKTECEKKCKGECDAYKKFIE
ECKGKADEGTSGSSWSKRWDQIYKRYSKYIEDAKRNRKAGTKNCGPSSTTSTAESKCVQS
>gi|254952598|gb|ACT97129.1| VAR2CSA [Plasmodium falciparum] |
334 aa
(SEQ ID NO: 14)
KCDKCKSEQSKKNNNIWIWKKSSGTEGGLQKEYANTIALPPRTQSLYLGNLRKLENVCEDVKDINFDTKEK
FLAGCLIAAFHEGKNLKKRYLEKKNGDNNSKLCKALKYSFADYGDLIKGTSIWDNEYTKDLELNLQKIFGK
LFRKYIKKNNTAEQHTSYSSLDELRESWWNTNKKYIWLAMKHGTTCSSGSGDNGSISCDDIPTIDLIPQYL
RFLQEWVEHFCEQRQGKVNAVIENCNSCKNTSSKTKLGGTCNGECKTECKGECDAYKEFIEKCKGTAAEGT
SGSSWVKRWYQIYMRYSKYIEDAKRNRKAGTKNCGTSSTTSTAESKCVQS
>gi|254952596|gb|ACT97128.1| VAR2CSA [Plasmodium falciparum] |
332 aa
(SEQ ID NO: 15)
KCDKCKSEQSKKNNNIWIWKKSSGTEGGLQKEYANTIALPPRTQSLYLGNLRKLENVCEDVKDINFDTKEK
FLAGCLIAAFHEGKNLKKRYLEKKNGDNNSKLCKALKYSFADYGDLIKGTSIWDNEYTKDLELNLQKIFGK
LFRKYIKKNNTAEQDTSYSSLDELRESWWNTNKKYIWTAMKHGTTCSSGSGDNGSISCDDIPTIDLIPQYL
RFLQEWVEHFCEQRQEKVKDVIKNCNSCKECGGTCNGECKTECKNKCKDECDAYKKFIEECEGKAAEGTSG
SSWSKRWDQIYKRYSKYIEDAKRNRKAGTKNCGTSSTTSTAENKCVQS
>gi|90193465|gb|ABD92328.1| erythrocyte membrane protein 1
[Plasmodium falciparum] | 267 aa
(SEQ ID NO: 16)
NYIKDDPYSAEYTTKLSFILNSSDTENASEKIQKNNDEVCNPNESGIACVELAQTSGSSSNKTCNTHSFIK
ANKKKVCKDVKLGINKKDKDLKICVIEDDSLRGVDNCCCQDLLGILQENCSDKNQSGSSSNGSCNNKNQEA
CQKKLENVFASLTNGYKCEKCKSEQSKKNNKNWIWKKYSVKEEGLQKEYANTIALPPRTQSLYLGNLPKLG
NVCKGVTDINFDTKEKFLAGCLIAAFHEGKNLKTTYLQNKKKLCKALKYSFADY
>gi|90193477|gb|ABD92334.1| erythrocyte membrane protein 1
[Plasmodium falciparum] | 263 aa
(SEQ ID NO: 17)
DYIKGDPYFAEYATKLSFILNSSDANTSSGETANHNDEACNPNESEIASVEQASISDRSSQKACNTHSSIK
ANKKKECKHVKLGVRENDKDLKICVIEDTSLSGVDNCCCQDLLGILQENCSDNKRGSSSNGSCDKNSEEIC
QKKLDEALASLHNGYKNQKCKSEQSKKNKNKWIWKKSSGNEKGLQKEYANTIGLPPRTQSLYLGNLPKLEN
VCEDVTDINFDTKEKFLAGCLIAAFHEGKNLKTTYPQNKNDDNGKKLCKD
>gi|254952594|gb|ACT97127.1| VAR2CSA [Plasmodium falciparum] |
338 aa
(SEQ ID NO: 18)
KCDKCKSEQSKKNNNIWIWKKSSGNKKGLQKEYANTIGLPPRTQSLYLGNLPKLENVCKDVTDINFDTKEK
FLAGCLIAAFHEGKNLKISNEKKNDDNGKKLCKDLKYSFADYGDLIKGTSIWDNEYTKDLELNLQNNFGKL
FRKYIKKNNTAEQHTLYSSLDELRESWWNTNKKYIWLAMKHGTTCSSGSGDNGDGSVTGSGSSCDDMSTID
LIPQYLRFLQEWVEHFCKQRQEKVNAVIENCNSCKNTSSKTKLGGTCNGECKTECEKKCKDECEKYKEFIE
ECKRGDGTAGSPWVKRWDQIYMRYSKYIEDAKRNRKAGTKSCGTSAAENKCVQS
>gi|254952602|gb|ACT97131.1| VAR2CSA [Plasmodium falciparum] |
341 aa
(SEQ ID NO: 19)
KCDKCKSEQSKKNNNIWIWKKSSGDEKGLQKEYANTIALPPRTQSLYLGNLPKLENVCKDVTDINFDTKEK
FLAGCLIAAFHEGKNLKTSHQNKNADNGKKNDDNGKKLCKALKYSFADYGDLIKGTSIWDNEYTKDLELNL
QQIFGKLFRKYIKRNNTAEQHTLYSSLDELRESWWNTNKKYIWLAMKHGTTCSSGSGDNGDGSVTGSGSSC
DDMSTIDLIPQYLRFLQEWVEHFCKQRQEKVKDVITNCNSCKECGGTCGSDCKTKCEAYKKFIEECNGTAD
GGTSGSSWSKRWDQIYKRYSKYIEDAKRNRKAGTKNCGPSSGANSGVTTTENKCVQS
>gi|254952660|gb|ACT97160.1| VAR2CSA [Plasmodium falciparum] |
352 aa
(SEQ ID NO: 20)
KCEKCESEQSKKNNKYWIWKKSSGNGEGLQEEYANTIALPPRTHSLCLVCLHEKEGKKTQELKNIRTNSEL
LKERIIAAFHEGKNLKTSPQNKNDNGKKLCKDLKYSFADYGDLIKGTSIWDNEYTKDLELNLQKIFGKLFR
KYIKKNNTAEQHTLYSSLDELRESWWNTNKKYIWLAMKHGAGMNSTMCNADGSVTGSSDSGSTTCCGDNGS
ISCDDMPTIDLIPQYLRFLQEWVEHFCEQRQEKVNAVITNCKSCKECGGTCNSDCEKKCKAYKEFIEKCKG
GGTEGTSGSSWSKRWDQIYKRHSKHIEDAKRNRKAGTKNCGITTGTISGESSGANSGVTTTENKCVQS
>gi|254952652|gb|ACT97156.1| VAR2CSA [Plasmodium falciparum] |
344 aa
(SEQ ID NO: 21)
KCDKCKSGTSRSRKIWTWRKFRGNGEGLQKEYANTIGLSPRTQLLYLVCLHEKGKKTQELKNISTNSELLK
EWIIAAFHEGKNLKTTYPQKKNDDNGKKLCKALKYSFADYGDLIKGTSIWDNDFTKDLELNLQKIFGKLFR
KYIKKNIASDENTSYSSLDELRESWWNTNKKYIWTAMKHGAGMNGTTCCGDGSVTGSSDSGSTTCCGDGSV
TGSGSSCDDIPTIDLIPQYLRFLQEWVEHFCEQRQEKVKDVITNCKSCKESEKKCKNKCDAYKEFIDGTGS
GGGTGTAGSSWSKRWDQIYMRYSKYIEDAKRNRKAGTKNCGTSSGANSGVTTTENKCVQS
>gi|254952622|gb|ACT97141.1| VAR2CSA [Plasmodium falciparum] |
350 aa
(SEQ ID NO: 22)
KCEKCKSEQSKKNNKIWTWRKFPGNGEGLQKEYANTIGLSPRTQLLYLVCLHEKGKKTQHKTISTNSELLK
EWIIAAFHEGKNLKKRYLEKKKGDNNSKLCKDLKYSFADYGDLIKGTSIWDNDFTKDLELNLQQIFGKLFR
KYIKKNIASDENTSYSSLDELRESWWNTNKKYIWTAMKHGAGMNSTMCNGDGSVTGSSDSGSTTCSGDNGS
ISCDDIPTIDLIPQYLRFLQEWVEHFCEQRQEKVKDVIKNCNSCKECGGTCNGECKTECKNKCKDECEKYK
NFIEVCTGGDGTAGSPWSKRWYQIYMRYSKYIEDAKRNRKAGTKSCGTSSGANSGVTTTESKCVQS
>gi|254952626|gb|ACT97143.1| VAR2CSA [Plasmodium falciparum] |
359 aa
(SEQ ID NO: 23)
KCEKCKSEQSKKNNKNWIWRKFPGNGEGLQKEYANTIGLPPRTHSLYLVCLHEKGKKTQELKNIRTNSELL
KEWIIAAFHEGKNLKKRYHQNNNSGNKKKLCKALEYSFADYGDLIKGTSIWDNEYTKDLELNLQQIFGKLF
RKYIKKNISTEQDTLYSSLDELRESWWNTNKKYIWLAMKHGAGMNSTTCCGDGSVTGSSDSGSTTCSGDNG
SISCDDMPTIDLIPQYLRFLQEWVEHFCEQRQEKVKDVIENCKSCKNTSGERIIGGTCNGECKTECEKKCK
AACEAYKTFIEECEGKAAEGTSGSSWSKRWYQIYMRYSKYIEDAKRNRKAGTKNCGKSSGANSGVTTTENK
CVQS
>gi|90193469|gb|ABD92330.1| erythrocyte membrane protein 1
[Plasmodium falciparum] | 270 aa
(SEQ ID NO: 24)
NYIKDDPYSKEYVTKLSFIPNSSDANNPSGETANHNDEVCNPNESEISSVEHAQTSVLLSQKAYITHSSIK
ANKKKVCKYVKLGVRENDKDLKICVIEDDSLRGVENCCFKDFLRILQENCSDNKRESSSNGSCNNNNEEAC
EKNLDEALASLTNCYKNQKCKSGTSTVNNNKWIWKKSSGKEGGLQKEYANTIGLPPRTQSLCLVVCLDEKE
GKTQELKNIRTNSELLKEWIIAAFHEGKNLKKRYHQNKNDDNNSKLCKALKYSFADY
>gi|254952644|gb|ACT97152.1| VAR2CSA [Plasmodium falciparum] |
334 aa
(SEQ ID NO: 25)
KCDKCKSEQSKKNNKYWIWKKYSVKEGGLQKEYANTIALPPRTQSLCLVVCLDEKEGKTQELKNIRTNSEL
LKERIIAAFHEGKNLKTYHEKKKGDDGKKLCKDLKYSFADYGDLIKGTSIWDNDFTKDLELNLQKIFGKLF
RKYIKKNNTAEQHTSYSSLDELRESWWNTNKKYIWTAMKHGAEMNGTTCSCSGDSSNDIPTIDLIPQYLRF
LQEWVEHFCEQRQAKVNAVIKNCKSCKECGGTCNGECKTECKTKCKGECEKYKEFIEKCEGQAAEGTSGSS
WSKRWYQIYMRYSKYIEDAKRNRKAGTKNCGTSSGANSGVTTTENKCVQS
>gi|254952642|gb|ACT97151.1| VAR2CSA [Plasmodium falciparum] |
351 aa
(SEQ ID NO: 26)
KCDKCKSEQSKKNNKNWIWKKYSGTEGGLQKEYANTIALPPRTQSLYLVCLHEKEEKTQELKNISTNSELL
KEWIIAAFHEGKNLKISPQNKNDNGKNLCKDLKYSFADYGDLIKGTSIWDNDFTKDLELNLQQIFGKLFRK
YIKKNNTAEQDTLYSSLDELRESWWNTNKKYIWTAMKHGAGMNGTTCCGDGSVTGSSDSGSTTCCGDGSVT
GSGSSCDDIPTIDLIPQYLRFLQEWVEHFCEQRQAKVKDVIKNCNSCKECGGTCNGECKTECEKKCKGECE
AYKKFIEKCNGGGGEGTSGSSWSKRWDQIYMRYSKYIEDAKRNRKAGTKNCGTSSTTNAAENKCVQS
>gi|254952658|gb|ACT97159.1| VAR2CSA [Plasmodium falciparum] |
353 aa
(SEQ ID NO: 27)
KCDKCKSGTSTVNKKWIWKKFPGKEGGLQEEYANTIALPPRTQSLCLVVCLDEKEGKTQHKTISTNSELLK
EWIIAAFHEGKNLKISNKKKNDENNSKLCKDLKYSFADYGDLIKGTSIWDNDFTKDLELNLQKIFGKLFRK
YIKKNNTAEQDTSYSSLDELRESWWNTNKKYIWLAMKHGTTCSSGSGDNGDGSVTGSSDSGSTTCCGDGSV
TGSGSSCDDIPTIDLIPQYLRFLQEWVEHFCKQRQAKVKDVIENCKSCKNTSSKTKLGDTCNSDCKTKCKV
ACEKYKEFIEKCVSAAGGTSGSSWVKRWDQIYMRYSKYIEDAKRNRKAGTKNCGPSSTTSTAESKCVQS
>gi|254952640|gb|ACT97150.1| VAR2CSA [Plasmodium falciparum] |
327 aa
(SEQ ID NO: 28)
KCDKCKSGTSTVNKKWIWKKYSGKEGGLQKEYANTIGLPPRTQSLCLVCLHEKEGKTQELKNISTNSELLK
EWIIAAFHEGKNLKISNKKKNDDNGKKLCKDLKYSFADYGDLIKGTSIWDNDFTKDLELNLQKIFGKLFRK
YIKKNNTAEQDTLYSSLDELRESWWNTNKKYIWTAMKHGAGMNSTTCSCSGDSSNDIPTIDLIPQYLRFLQ
EWVEHFCKQRQEKVNAVITNCKSCKESGGTCNSDCEKKCKIECEKYKNFIEKCVTAAGGTSGSSWSKRWDQ
IYKMYSKYIEDAKRNRKAGTKNCGPSSTTNAAASTDENKCVQS
>dd2full 745 amino acids | 628 aa
(SEQ ID NO: 29)
NYIKGDPYFAEYATKLSFILNSSDTENASETPSKYYDEACNCNESEIASVGQAQTSGPSSNKTCITHSSIK
TNKKKECKDVKLGINNNDKVLRVCVIEDTSLSGVDNCCCQDLLGILQENCSDNKRGSSSNGSCDKNSEEIC
QKKLEKVFASLTNGYKCDKCKSGTSRSKKKWIWKKSSGNEEGLQKEYANTIGLPPRTQSLCLVCLHEKEGK
TQHKTISTNSELLKEWIIAAFHEGKNLKTSHEKKNDDNGKKLCKALEYSFADYGDLIKGTSIWDNEYTKDL
ELNLQKIFGKLFRKYIKKNNTAEQHTSYSSLDELRESWWNTNKKYIWTAMKHGAGMNGTTCSCSGDSSNDM
PTIDLIPQYLRFLQEWVEHFCKQRQEKVNAVIENCNSCKESGGTCNSDCKTECKNKCEAYKEFIEDCKGGG
TGTAGSPWSKRWDQIYKRYSKHIEDAKRNRKAGTKNCGTSSTTNAAASTDENKCVQSDVDSFFKHLIDIGL
TTPSSYLSNVLDDNICGADKAPWTTYTTYTTTKNCDIQKKTPKSQSCDTLVVVNVPSPLGNTPHEYKYACE
CKIPTTEETCDDRKEYMNQWSCGSAQTVRGRSGKDDYELYTYNGVKETKPLGTLKNSKLD
>gi|254952636|gb|ACT97148.1| VAR2CSA [Plasmodium falciparum] |
350 aa
(SEQ ID NO: 30)
KCEKCKSEQSKKNNKNWIWRKFRGTEGGLQEEYANTIGLPPRTQSLCLVVCLDEKGKKTQELKNIRTNSEL
LKEWIIAAFHEGKNLKPSHQNKNSGNKENLCKALKYSFADYGDLIKGTSIWDNDFTKDLELNLQKIFGKLF
RKYIKKNNTAEQHTSYSSLDELRESWWNTNKKYIWTAMKHGAEMNGTTCNADGSVTGSSDSGSTTCSGDNG
SISCDDIPTIDLIPQYLRFLQEWVEHFCKQRQEKVNAVINSCNSCKNTSSKTKLGDTCNSDCKTKCKIECE
KYKTFIEKCVTAAGGTSGSPWSKRWDQIYKRYSKYIEDAKRNRKAGTKNCGPSSTTSTAESKCVQS
>gi|254952638|gb|ACT97149.1| VAR2CSA [Plasmodium falciparum] |
330 aa
(SEQ ID NO: 31)
KCDKCKSEQSKKNNKNWIWRKYSGNGEGLQKEYANTIGLPPRTHSLYLVCLHEKEGKTQELKNIRTNSELL
KEWIIAAFHEGKNLKTTYLENKNDENKKKLCKALKYSFADYGDLIKGTSIWDNDFTKDLELNLQKIFGKLF
RKYIKKNIASDENTLYSSLDELRESWWNTNKKYIWTAMKHGAEMNGTTCSSGSGDNGSISCDDIPTIDLIP
QYLRFLQEWVGHFCKQRQEKVNAVITNCNSCKESGGTCNSDCEKKCKIECEKYKKFIEECRTAAGGTSGSP
WSKRWDQIYKMYSKYIEDAKRNRKAGTKNCGPSSTTSTAESKCVQS
>gi|254952628|gb|ACT97144.1| VAR2CSA [Plasmodium falciparum] |
334 aa
(SEQ ID NO: 32)
KCDKCKSEQSKKNNKNWIWRKYSGNGEGLQKEYANTIGLPPRTHSLYLVCLHEKEGKTQHKTISTNSELLK
EWIIAAFHEGKNLKKRYPQNNNSGNKKKLCKDLKYSFADYGDLIKGTSIWDNEYTKDLELNLQKAFGKLFR
KYIKKNIASDENTLYSSLDELRESWWNTNKKYIWLAMKHGAEMNGTMCNADGSVTGSGSSCDDMSTIDLIP
QYLRFLQEWVEHFCEQRQAKVKDVINSCKSCKESGDTCNSDCEKKCKNKCDAYKTFIEEFCTADGGTAGSP
WSKRWDQIYKRYSKYIEDAKRNRKAGTKNCGTSSGANSGVTTTENKCVQS
>gi|254952630|gb|ACT97145.1| VAR2CSA [Plasmodium falciparum] |
350 aa
(SEQ ID NO: 33)
KCDKCKSGTSTVNKNWIWKKYSGKEEGLQKEYANTIALPPRTHSLYLVCLHEKGKKTQELKNIRTNSELLK
EWIIAAFHEGKNLKTSPQNNNSGNKKKLCKALKYSFADYGDLIKGTSIWDNDFTKDLELNLQKIFGKLFRK
YIKKNNTAEQHTSYSSLDELRESWWNTNKKYIWLAMKHGAEMNGTTCCGDGSVTGSSDSGSTTCSGDNGSI
SCDDMPTTDFIPQYLRFLQEWVEHFCKQRQEKVKHVMESCKSCKECGDTCNGECKTECEKKCKNKCEAYKT
FIEKCVSADGGTSGSSWSKRWDQIYMRYSKYIEDAKRNRKAGTKNCGTSSTTNAAASTAENKCVQS
>P13 745 amino acids | 647 aa
(SEQ ID NO: 34)
DYIKDDPYSAEYATKLSFILNPSDANTSSGETANHNDEVCNCNESEIASVELAPISDSSSNKTCITHSFIG
ANKKKECKDVKLGVREKDKDLKICVIEDDSLRGVENCCCQDLLGILQENCSDNKSGSSSNGSCDKNSEDEC
QKKLENVFASLKNGYKCDKCKSGTSTVNKKWIWRKYSGNGEGLQKEYANTIGLPPRTHSLYLVCLHEKEGK
TQHKTISTNSELLKEWIIAAFHEGKNLKTSHQNNNSGNKKKLCKALKYSFADYGDLIKGTSIWDNDFTKDL
ELNLQKIFGKLFRKYIKKNIASDENTSYSSLDELRESWWNTNKKYIWLAMKHGAEMNSTMCNGDGSVTGSS
DSGSTTCSGDNGSISCDDIPTIDLIPQYLRFLQEWVEHFCKQRQEKVKDVITNCKSCKESGDTCNSDCEKK
CKNKCEAYKKFIEERRTAAQGTAESSWVKRWDQIYMRYSKYIEDAKRNRKAGTKSCGPSSTTNAAASTAEN
KCVQSDIDSFFKHLIDIGLTTPSSYLSIVLDDNICGADNAPWTTYTTYTTTKNCDIKKKTPKPQSCDTLVV
VNVPSPLGNTPHEYKYACQCRTPNKQESCDDRKEYMNQWSSGSAQTVRGRSTNNDYELYTYNGVKETKPLG
TLKNSKLD
>gi|254952608|gb|ACT97134.1| VAR2CSA [Plasmodium falciparum] |
341 aa
(SEQ ID NO: 35)
KCDKCKSGTSTVNKKWIWRKSSGNKEGLQKEYANTIGLPPRTQSLYLGNLPKLENVCEDVKDINFDTKEKF
LAGCLIVSFHEGKNLKTSHEKKNDDNGKKLCKALEYSFADYGDLIKGTSIWDNEYTKDLELNLQKIFGKLF
RKYIKKNNTAEQDTSYSSLDELRESWWNTNKKYIWTAMKHGAGMNITTCCGDGSSGENQTNSCDDIPTIDL
IPQYLRFLQEWVEHFCKQRQEKVNAVVTNCKSCKESGGTCNGECKTKCKNKCEVYKTFIDNVGDGTAGSPW
VKRWDQIYKRYSKHIEDAKRNRKAGTKNCGITTGTISGESSGATSGVTTTENKCVQS
>7g8 745 amino acids I 632 aa
(SEQ ID NO: 36)
NYIKDDPYSKEYVTKLSFIPNSSDANTSSEKIQKNNDEVCNPNESGISSVEQAQTSGPSSNKTCITHSSIK
ANKKKECKDVKLGVRENDKDLKICVIEDTSLSGVDNCCCQDLLGILQENCSDNKRGSSSNDSCDNKNQDEC
QKKLDEALESLHNGYKNQKCKSGTSTVNKKWIWKKSSGNKEGLQKEYANTIGLPPRTQSLYLGNLPKLENV
SKGVTDIIYDTKEKFLAGCLIVSFHEGKNLKTSHEKKNDDNGKKLCKALEYSFADYGDLIKGTSIWDNEYT
KDLELNLQKAFGKLFRKYIKKNISAEQDTSYSSLDELRESWWNTNKKYIWIAMKHGAGMNGTTCCGDGSSG
ENQTNSCDDIPTIDLIPQYLRFLQEWVEHFCEQRQAKVKDVITNCKSCKNTSGERKIGGTCNGECKTKCKN
KCEAYKTFIEHCKGGDGTAGSSWVKRWDQIYKRYSKHIEDAKRNRKAGTKSCGTSTAENKCVQSDIDSFFK
HLIDIGLTTPSSYLSIVLDENNCGEDKAPWTTYTTTKNCDIQKDKSKSQSSDTLVVVNVPSPLGNTPHGYK
YACQCKIPTTEETCDDRKEYMNQWSCGSARTMKRGYKNDNYELCKYNGVDVKPTTVRSSSTKLD
>Indo 745 amino acids | 639 aa
(SEQ ID NO: 37)
DYIKGDPYSAEYVTKLSFIPNSSDANNPSEKIQKNNDEVCNCNESEISSVGQASISDPSSNKTCNTHSSIK
ANKKKVCKDVKLGVRENDKVLKICVIEHTSLRGVDNCCFKDLLGILQEPRIDKNQSGSSSNGSCDKNSEEA
CEKNLEKVLASLTNGYKCDKCKSGTSRSKKKWIWKKYSGKEGGLQEEYANTIGLPPRTQSLCLVVCLDEKE
GKTQELKNISTNSELLKEWIIAAFPEGKNLKPSPEKKKGDNGKKLCKDLKYSFADYGDLIKGTSIWDNEYT
KDLELNLQKIFGKLFRKYIKKNIASDENTLYSSLDELRESWWNTNKKYIWLAMKHGAGMNSTMCNADGSVT
GSGSSCDDMPTIDLIPQYLRFLQEWVEHFCKQRQEKVKPVIENCNSCKNTSSERKIGGTCNSDCKTECKNK
CEVYKKFIEDCKGGDGTAGSSWSKRWDQIYKRYSKYIEDAKRNRKAGTKNCGPSSTTNAAENKCVQSDIDS
FFKHLIDIGLTTPSSYLSTVLDDNICGEDNAPWTTYTTYTTTKNCDKDKKKSKSQSCDTLVVVNVPSPLGN
TPHEYKYACECRTPNKQESCDDRKEYMNQWISDNTKNPKGSGSGKDYYELYTYNGVDVKPTTVRSSSTKLD
>MC 745 amino acids | 655 aa
(SEQ ID NO: 38)
DYIKGDPYFAEYATKLSFILNSSDANTSSGETANHNDEACNCNESEISSVEHASISDPSSNKTCNTHSSIK
ANKKKVCKHVKLGVRENDKDLRVCVIEHTSLSGVENCCFKDFLRILQENCSDNKSGSSSNGSCDKNNEEAC
EKNLEKVFASLTNCYKCEKCKSEQSKKNNKKWTWRKSSGNKGGLQEEYANTIGLPPRTQSLCLVVCLDEKE
GKKTQELKNIRTNSELLKEWIIAAFHEGKNLKPSHEKKNDDNGKKNDDNNSKLCKDLKYSFADYGDLIKGT
SIWDNEYTKDLELNLQKIFGKLFRKYIKKNIASDENTLYSSLDELRESWWNTNKKYIWLAMKHGAEMNGTT
CNADGSVTGSGSSCDDIPTIDLIPQYLRFLQEWVEHFCKQRQAKVKDVIENCKSCKESGNKCKTECKNKCE
AYKKFIENCKGGDGTAGSSWVKRWDQIYMRYSKYIEDAKRNRKAGTKNCGPSSITNVSASTDENKCVQSDI
DSFFKHLIDIGLTTPSSYLSIVLDDNICGDDKAPWTTYTTYTTYTTYTTYTTYTTYTTTKNCDKERDKSKS
QSCNTAVVVNVPSPLGNTPHEYKYACECRTPSNKELCDDRKEYMNQWSSGSAQTVRDRSGKDYYELYTYNG
VKETKLPKKLNSSKLD
>gi|254952650|gb|ACT97155.1| VAR2CSA [Plasmodium falciparum] |
347 aa
(SEQ ID NO: 39)
KCDKCKSEQSKKNNKYWIWKKSSVKEEGLQKEYANTIALPPRTHSLCLVVCLDEKGKKTQELKNISTNSEL
LKERIIAAFHEGKNLKTTYLEKKNADNNSKLCKALKYSFADYGDLIKGTSIWDNEYTKDLELNLQQIFGKL
FRKYIKKNNTAEQHTLYSSLDELRESWWNTNKKYIWLAMKHGAGMNGTTCCGDGSVTGSSDSGSTTCSGDN
GSISCDDMPTTDFIPQYLRFLQEWVEHFCKQRQEKVKDVIENCNSCKNNLGKTEINEKCKTECKNKCEAYK
NFIEKFCTADGGTSGSPWSKRWDQIYKRYSKYIEDAKRNRKAGTKNCGTSSTTSTAENKCVQS
>gi|254952648|gb|ACT97154.1| VAR2CSA [Plasmodium falciparum] |
335 aa
(SEQ ID NO: 40)
KCEKCKSGTSTVNKYWIWRKSSGNKEGLQKEYANTIALPPRTHSLCLVVCLDEKEGKTQELKNISTNSELL
KERIIAAFHEGENLKTSHEKKKGDDGKKNADNNSKLCKALKYSFADYGDLIKGTSIWDNEYTKDLELNLQK
IFGKLFRKYIKKNIASDENTSYSSLDELRESWWNTNKKYIWLAMKHGAGMNGTTCSCSGDSSDDMPTTDFI
PQYLRFLQEWVEHFCKQRQENVNAVIENCNSCKECGGTCNSDCEKKCKTECKNKCEAYKNFIEKFCTADGG
TSGYSWSKRWDQIYKRYSKYIEDAKRNRKAGTKSCGTSSTTSTAESKCVQS
>ghana2 745 amino acids | 667 aa
(SEQ ID NO: 41)
SYVKNNPYSKEYVTKLSFILNPSDANNPSETPSKYYDEVCNCNESGIACVGQAQTSGPSSNKTCITHSFIG
ANKKKVCKDVKLGVREKDKDLKICVIEDTYLSGVDNCCFKDFLGMLQENCSDNKSGSSSNGSCNNKNQDEC
EKNLDEALASLTNGYKCEKCKSGTSTVNKYWIWRKSSGNKEGLQKEYANTIALPPRTHSLCLVVCLDEKEG
KTQHKTISTNSELLKEWIIAAFHEGKNLKTSHEKKKGDDGKKNADNNSKLCKALKYSFADYGDLIKGTSIW
DNDFTKDLELNLQKIFGKLFRKYIKKNIASDENTSYSSLDELRESWWNTNKKYIWLAMKHGAGMNSTTCCG
DGSVTGSSDSGSTTCCGDGSVTGSGSSCDDMPTTDFIPQYLRFLQEWVEHFCKQRQENVNAVIENCNSCKE
CGGTCNSDCEKKCKTECKGECDAYKEFIEKCNGGAAEGTSGSSWSKRWDQIYKRYSKYIEDAKRNRKAGTK
NCGTSSTTSTAESKCVQSDIDSFFKHLIDIGLTTPSSYLSIVLDENICGADNAPWTTYTTYTTYTTYTTTE
KCNKETDKSKLQQCNTSVVVNVPSPLGNTPHGYKYVCECRTPNKQETCDDRKEYMNQWISDNTKNPKGSRS
TNNDYELYTYNGVQIKPTTVRSNSTKLD
>gi|254952634|gb|ACT97147.1| VAR2CSA [Plasmodium falciparum] |
348 aa
(SEQ ID NO: 42)
KCDKCKSEQSKKNNKNWIWKKSSGNEKGLQKEYANTIGLPPRTQSLCLVVCLDEKEGKTQELKNIRTNSEL
LKEWIIAAFHEGKNLKTSHEKKKGDNNSKLCKDLKYSFADYGDLIKGTSIWDNEYTKDLELNLQNNFGKLF
RKYIKKNIASDENTSYSSLDELRESWWNTNKKYIWLAMKHGAGMNSTTCSSGSGSTTCSSGSGSTTCSSGS
GDSCDDMPTIDLIPQYLRFLQEWVEHFCKQRQEKVNAVIKNCNSCKESGGTCNGECKTECKNKCEAYKTFI
EEFCTADGGTSGSPWSKRWDQIYKMYSKHIEDAKRNRKAGTKNCGPSSTTNVSVSTDENKCVQS
>ghana1 745 amino acids | 652 aa
(SEQ ID NO: 43)
DYIKDDPYFAEYVTKLSFILNSSDANNPSGETANHNDEVCNPNESGIASVEQAQTSDPSSNKTCNTHSSIK
ANKKKVCKHVKLGVRENDKDLKICVIEHTSLSGVENCCCQDFLRILQENCSDNKSGSSSNGSCNNKNQEAC
EKNLEKVLASLTNCYKCDKCKSEQSKKNNKNWIWKKSSGNEKGLQKEYANTIGLPPRTQSLCLVVCLDEKE
GKTQELKNIRTNSELLKEWIIAAFHEGKNLKKRYPQNKNDDNNSKLCKDLKYSFADYGDLIKGTSIWDNEY
TKDLELNLQNNFGKLFRKYIKKNISTEQDTLYSSLDELRESWWNTNKKYIWLAMKHGAGMNSTTCSSGSGS
TTCSSGSGSTTCSSGSGDSCDDMPTTDFIPQYLRFLQEWVEHFCKQRQEKVNAVIKNCNSCKESGGTCNGE
CKTECKNKCEAYKTFIEEFCTADGGTSGSPWSKRWDQIYKMYSKHIEDAKRNRKAGTKNCGPSSTTNVSVS
TDENKCVQSDIDSFFKHLIDIGLTTPSSYLSIVLDDNICGEDKAPWTTYTTYTTTKKCNKETDKSKSQSCN
TAVVVNVPSPLGNTPHGYKYACECKIPTTEETCDDRKEYMNQWIIDTSKKQKGSGSGKDDYELYTYNGVDV
KPTTVRSNSTKLD
>V1S1 745 amino acids | 628 aa
(SEQ ID NO: 44)
DYIKDDPYSAQYTTKLSFILNPSDANTSSEKIQKNNDEACNCNESGISSVGQAQTSGPSSNKTCITHSSIK
ANKKKVCKDVKLGINNNDKVLRVCVIEDTSLSGVDNCCCQDLLGILQENCSDNKRGSSSNGSCNNNNEEAC
EKNLDEAPASLHNGYKNQKCKSGTSRSKKKWIWKKSSGNEKGLQEEYANTIGLPPRTQSLCLVCLHEKEGK
TQHKTISTNSELLKEWIIAAFHEGKNLKTSHEKKNDDNGKKLCKALEYSFADYGDLIKGTSIWDNEYTKDL
ELNLQKAFGKLFRKYIKKNNTAEQDTSYSSLDELRESWWNTNKKYIWIAMKHGAGMNGTTCSCSGDSSNDM
PTIDLIPQYLRFLQEWVEHFCEQRQAKVKDVITNCKSCKESGNKCKTECKTKCKDECEKYKTFIEDCNGGG
TGTAGSSWVKRWDQIYKRYSKHIEDAKRNRKAGTKNCGPSSITNAAASTDENKCVQSDIDSFFKHLIDIGL
TTPSSYLSNVLDENSCGDDKAPWTTYTTYTTTKNCDIQKDKSKSQPINTSVVVNVPSPLGNTPYRYKYACE
CKIPTTEESCDDRKEYMNQWSCGSARTMKRGYKNDNYELCKYNGVDVKPTTVRSNSSKLD
>raj116_var25 745 amino acids | 653 aa
(SEQ ID NO: 45)
DYIKGDPYFAEYATKLSFILNPSDTENASETPSKYYDEACNPNESEIASVEQAQTSGPSSNKTCITHSSIK
TNKKKECKDVKLGVRENDKDLKICVIEDTSLSGVDNCCFKDLLGILQENCSDNKRGSSSNDSCNNNNEEAC
EKNLDEALASLTNGYKCDKCKSGTSTVNKKWTWRKSSGNEEGLQKEYANTIGLPPRTQSLCLVCLHEKEGK
TKHKTISTNSELLKEWIIAAFHEGKNLKTSHEKKNDDNGKKLCKALEYSFADYGDLIKGTSIWDNEYTKDL
ELNLQKAFGKLFRKYIKKNNTAEQDTSYSSLDELRESWWNTNKKYIWTAMKHGAEMNGTTCSSGSGDNGDS
SITGSSDSGSTTCSGDNGSISCDDIPTTDFIPQYLRFLQEWVEHFCEQRQAKVKDVINSCNSCNESGGTCN
GECKTKCKDECEKYKKFIEDCNGGDGTAGSSWVKRWDQIYKRYSKHIEDAKRNRKAGTKNCGPSSITNAAA
STDENKCVQSDVDSFFKHLIDIGLTTPSSYLSIVLDENSCGDDKAPWTTYTTYTTTEKCNKERDKSKSQSS
DTLVVVNVPSPLGNTPHEYKYACECKIPTNEETCDDRKDYMNQWISDTSKKQKGSGSGKDYYELYTYNGVQ
IKQAAGRSSSTKLD
>gi|31323048|gb|AAP37940.1| var2csa [Plasmodium falciparum] |
490 aa
(SEQ ID NO: 46)
KCDKCKSEQSKKNNNKWIWKKYSGNGEGLQKEYANTIGLPPRTQSLCLVCLHEKEGKTQHKTISTNSELLK
EWIIAAFHEGKNLKKRYPQNKNDDNNSKLCKALEYSFADYGDLIKGTSIWDNEYTKDLELNLQKAFGKLFR
KYIKKNNTAEQDTSYSSLDELRESWWNTNKKYIWTAMKHGAEMNGTTCSSGSGDNGDSSCDDIPTIDLIPQ
YLRFLQEWVEHFCKQRQAKVKDVINSCNSCKNTSGERKIGGTCNSDCEKKCKVACDAYKTFIEECRTAVGG
TAGSSWVKRWDQIYKRYSKHIEDAKRNRKAGTKNCGPSSTTNAAENKCVQSDIDSFFKHLIDIGLTTPSSY
LSNVLDENSCGADKAPWTTYTTYTTYTTYTTYTTTEKCNKERDKSKSQQSNTSVVVNVPSPLGNTPHEYKY
ACECKIPTTEETCDDRKEYMNQWIIDNTKNPKGSGSTDNDYELYTYNGVQIKQAAGRSSSTKLD
>gi|254952620|gb|ACT97140.1| VAR2CSA [Plasmodium falciparum] |
335 aa
(SEQ ID NO: 47)
KCEKCKSGTSTVNNKWIWRKSSGKEGGLQKEYANTIGLPPRTQSLYLGNLPKLENVCKGVTDIIYDTKEKF
LSGCLIAAFHEGKNLKTTYLEKKNDDNGKKLCKALEYSFADYGDLIKGTSIWDNEYTKDLELNLQKIFGKL
FRKYIKKNNTAEQDTSYSSLDELRESWWNTNKKYIWIAMKHGAGMNGTTCSSGSGDSSNDIPTTDFIPQYL
RFLQEWVENFCEQRQAKVKPVIENCNSCKESGGTCNGECKTKCKVACDAYKKFIDGTGSGGGSRPTGIAGS
SWSKRWDQIYKRYSKHIEDAKRNRKAGTKNCGPSSITNVSVSTDENKCVQS
>T2C6 745 amino acids | 637 aa
(SEQ ID NO: 48)
NYIKDDPYSKEYVTKLSFIPNSSDANTSSEKIQKNNDEVCNPNESGISSVEQAQTSDPSSNKTCITHSSIK
ANKKKECKDVKLGVRENDKDLKICVIEHTSLSGVDNCCFKDFLRMLQEPRIDKNQRGSSSNGSCDKNSEEA
CEKNLDEALASLTNGYKCDKCKSEQSKKNNNKWIWKKFPGKEGGLQEEYANTIGLPPRTQYLCLVVCLDEK
EGKTQELKNIRTNSELLKEWIIAAFHEGKNLKTTYPQKKNDDNGKKLCKDLKYSFADYGDLIKGTSIWDNE
YTKNVELNLQNNFGKLFRKYIKKNNTAEQDTSYSSLDELRESWWNTNKKYIWLAMKHGAEMNSTTCCGDGS
VTGSGSSCDDIPTIDLIPQYLRFLQEWVEHFCKQRQAKVKDVITNCNSCKESGNKCKTECKNKCKDECEKY
KKFIEACGTAVGGTGTAGSPWSKRWDQIYKRYSKHIEDAKRNRKAGTKNCGPSSTTNAAENKCVQSDIDSF
FKHLIDIGLTTPSSYLSIVLDDNICGADKAPWTTYTTYTTENCDIQKKTPKSQSCDTLVVVNVPSPLGNTP
HGYKYACQCRTPNKQESCDDRKEYMNQWIIDNTKNPKGSGSGKDYYELCKYNGVKETKPLGTLKNSKLD
>gi|254952632|gb|ACT97146.1| VAR2CSA [Plasmodium falciparum] |
330 aa
(SEQ ID NO: 49)
KCDKCKSEQSKKNNNKWIWRKFPGKEGGLQKEYANTIGLPPRTQSLCLVCLHEKEGKTQHKTISTNSELLK
EWIIAAFHEGKNLKTTYLEKKNAENKKKLCKALKYSFADYGDLIKGTSIWDNEYTKDLELNLQKIFGKLFR
KYIKKNNTAEQDTSYSSLDELRESWWNTNKKYIWTAMKHGAGMNGTMCNADGSVTGSGSSCDDMPTTDFIP
QYLRFLQEWVEHFCKQRQAKVKDVIENCKSCKESGNKCKTECKNKCDAYKTFIEECGTAVGGTAGSSWVKR
WDQIYKRYSKHIEDAKRNRKAGTKNCGTSSTTNAAASTAENKCVQS
>gi|90193487|gb|ABD92339.1| erythrocyte membrane protein 1
[Plasmodium falciparum] | 269 aa
(SEQ ID NO: 50)
NYIKDDPYSKEYVTKLSFILNSSDAENASETPSKYYDEACNCNESGISSVEQASISDRSSQKACNTHSFIG
ANKKKVCKHVKLGVRENDKDLKICVIEDDSLRGVENCCFKDFLRMLQEPRIDKNQRGSSSNDSCNNNNEEA
CEKNLDEALASLHNGYKNQKCKSEQSKKNNNKWIWKKSSGKEGGLQKEYANTIGLPPRTQSLCLVCLHEKE
GKTQHKTISTNSELLKEWIIDAFHEGKNLKTTYLEKKKGDNGKKLCKALKYSFADY
>gi|254952646|gb|ACT97153.1| VAR2CSA [Plasmodium falciparum] |
347 aa
(SEQ ID NO: 51)
KCDKCKSEQSKKNNKNWIWKKSSGKEGGLQKEYANTIALPPRTQSLCLVVCLHEKEGKTQHKTISTNSELL
KEWIIDAFHEGKNLKTTYLEKQNADNGKKNADNNSKLCKDLKYSFADYGDLIKGTSIWDNEYTKDLELNLQ
QIFGKLFRKYIKKNIASDENTLYSSLDELRESWWNTNKKYIWTAMKHGAEMNGTTCSSGSGDSSSGENQTN
SCDDIPTIDLIPQYLRFLQEWVEHFCEQRQAKVKDVITNCKSCKESGGTCNSDCKTKCKGECEKYKKFIEK
CKGGGTEGTSGSSWVKRWYQIYMRYSKYIEDAKRNRKAGTKSCGTSSGANSGVTTTESKCVQS
>gi|90193485|gb|ABD92338.1| erythrocyte membrane protein 1
[Plasmodium falciparum] | 269 aa
(SEQ ID NO: 52)
DYIKDDPYSKEYTTKLSFILNSSDANTSSEKIQKNNDEVCNPNESEISSVEQAQTSRPSSNKTCITHSSIK
ANKKKVCKDVKLGVRENDKVLRVCVIEHTSLSGVENCCCQDLLGILQENCSDNKRGSSSNGSCDKNSEEAC
EKNLDEALASLTNCYKNQKCKSEQSKKNNNKWIWKKSSGNEKGLQKEYANTIGLPPRTQSLCLVCLHEKEG
KTQELKNISTNSELLKEWIIAAFHEGKNLKTTYPQNKNDDNGKKLFKDLKYSFADY
>MTS1 745 amino acids | 646 aa
(SEQ ID NO: 53)
DYIKDDPYSKEYTTKLSFILNSSDANTSSEKIQKNNDEVCNPNESEISSVEQAQTSRPSSNKTCITHSSIK
ANKKKVCKDVKLGVRENDKVLRVCVIEHTSLSGVENCCCQDLLGILQENCSDNKRGSSSNGSCDKNSEEAC
EKNLDEALASLTNCYKNQKCKSEQSKKNNNKWIWKKSSGKEGGLQKEYANTIGLPPRTQSLYLGNLPKLEN
VCKGVTDINFDTKEKFLAGCLIAAFHEGKNLKTTYLEKKNDDNGKKLCKALEYSFADYGDLIKGTSIWDNE
YTKDLELNLQKAFGKLFRKYIKKNNTAEQDTSYSSLDELRESWWNTNKKYIWTAMKHGAGMNGTTCSSGSG
DSSNDIPTTDFIPQYLRFLQEWVENFCEQRQAKVKDVIENCNSCKNTSGERKIGDTCNSDCEKKCKDECEK
YKKFIEDCKGGDGTAGSSWVKRWDQIYKRYSKHIEDAKRNRKAGTKNCGITTGTISGESSGATSGVTTTEN
KCVQSDIDSFFKHLIDIGLTTPSSYLSNVLDDNICGEDNAPWTTYTTYTTEKCNKETDKSKSQQSNTAVVV
NVPSPLGNTPHGYKYACECKIPTTEETCDDRKEYMNQWSCGSAQTVRDRSGKDDYELCKYNGVQIKQAAGT
LKNSKLD
>Q8I639 (Q8I639_PLAF7) Plasmodium falciparum (isolate 3D7),
632 aa extracellular part
(SEQ ID NO: 54)
NYIKGDPYFAEYATKLSFILNSSDANNPSEKIQKNNDEVCNCNESGIASVEQEQISDPSSNKTCITHSSIK
ANKKKVCKHVKLGVRENDKDLRVCVIEHTSLSGVENCCCQDFLRILQENCSDNKSGSSSNGSCNNKNQEAC
EKNLEKVLASLTNCYKCDKCKSEQSKKNNKNWIWKKSSGKEGGLQKEYANTIGLPPRTQSLCLVVCLDEKG
KKTQELKNIRTNSELLKEWIIAAFHEGKNLKPSHEKKNDDNGKKLCKALEYSFADYGDLIKGTSIWDNEYT
KDLELNLQKIFGKLFRKYIKKNNTAEQDTSYSSLDELRESWWNTNKKYIWLAMKHGAGMNSTTCCGDGSVT
GSGSSCDDIPTIDLIPQYLRFLQEWVEHFCKQRQEKVKPVIENCKSCKESGGTCNGECKTECKNKCEVYKK
FIEDCKGGDGTAGSSWVKRWDQIYKRYSKYIEDAKRNRKAGTKNCGPSSTTNAAENKCVQSDIDSFFKHLI
DIGLTTPSSYLSIVLDDNICGADKAPWTTYTTYTTTEKCNKETDKSKLQQCNTAVVVNVPSPLGNTPHGYK
YACQCKIPTNEETCDDRKEYMNQWSCGSARTMKRGYKNDNYELCKYNGVDVKPTTVRSNSSKLD
>Q8I639 (Q8I639_PLAF7) Plasmodium falciparum (isolate 3D7),
complete 2730 aa extracellular part
(SEQ ID NO: 55)
MDKSSIANKIEAYLGAKSDDSKIDQSLKADPSEVQYYGSGGDGYYLRKNICKITVNHSDSGTNDPCDRIPP
PYGDNDQWKCAIILSKVSEKPENVFVPPRRQRMCINNLEKLNVDKIRDKHAFLADVLLTARNEGERIVQNH
PDTNSSNVCNALERSFADIADIIRGTDLWKGTNSNLEQNLKQMFAKIRENDKVLQDKYPKDQNYRKLREDW
WNANRQKVWEVITCGARSNDLLIKRGWRTSGKSNGDNKLELCRKCGHYEEKVPTKLDYVPQFLRWLTEWIE
DFYREKQNLIDDMERHREECTSEDHKSKEGTSYCSTCKDKCKKYCECVKKWKSEWENQKNKYTELYQQNKN
ETSQKNTSRYDDYVKDFFKKLEANYSSLENYIKGDPYFAEYATKLSFILNSSDANNPSEKIQKNNDEVCNC
NESGIASVEQEQISDPSSNKTCITHSSIKANKKKVCKHVKLGVRENDKDLRVCVIEHTSLSGVENCCCQDF
LRILQENCSDNKSGSSSNGSCNNKNQEACEKNLEKVLASLTNCYKCDKCKSEQSKKNNKNWIWKKSSGKEG
GLQKEYANTIGLPPRTQSLCLVVCLDEKGKKTQELKNIRTNSELLKEWIIAAFHEGKNLKPSHEKKNDDNG
KKLCKALEYSFADYGDLIKGTSIWDNEYTKDLELNLQKIFGKLFRKYIKKNNTAEQDTSYSSLDELRESWW
NTNKKYIWLAMKHGAGMNSTTCCGDGSVTGSGSSCDDIPTIDLIPQYLRFLQEWVEHFCKQRQEKVKPVIE
NCKSCKESGGTCNGECKTECKNKCEVYKKFIEDCKGGDGTAGSSWVKRWDQIYKRYSKYIEDAKRNRKAGT
KNCGPSSTTNAAENKCVQSDIDSFFKHLIDIGLTTPSSYLSIVLDDNICGADKAPWTTYTTYTTTEKCNKE
TDKSKLQQCNTAVVVNVPSPLGNTPHGYKYACQCKIPTNEETCDDRKEYMNQWSCGSARTMKRGYKNDNYE
LCKYNGVDVKPTTVRSNSSKLDDKDVTFFNLFEQWNKEIQYQIEQYMTNTKISCNNEKNVLSRVSDEAAQP
KFSDNERDRNSITHEDKNCKEKCKCYSLWIEKINDQWDKQKDNYNKFQRKQIYDANKGSQNKKVVSLSNFL
FFSCWEEYIQKYFNGDWSKIKNIGSDTFEFLIKKCGNDSGDGETIFSEKLNNAEKKCKENESTNNKMKSSE
TSCDCSEPIYIRGCQPKIYDGKIFPGKGGEKQWICKDTIIHGDTNGACIPPRTQNLCVGELWDKRYGGRSN
IKNDTKESLKQKIKNAIQKETELLYEYHDKGTAIISRNPMKGQKEKEEKNNDSNGLPKGFCHAVQRSFIDY
KNMILGTSVNIYEYIGKLQEDIKKIIEKGTTKQNGKTVGSGAENVNAWWKGIEGEMWDAVRCAITKINKKQ
KKNGTFSIDECGIFPPTGNDEDQSVSWFKEWSEQFCIERLQYEKNIRDACTNNGQGDKIQGDCKRKCEEYK
KYISEKKQEWDKQKTKYENKYVGKSASDLLKENYPECISANFDFIFNDNIEYKTYYPYGDYSSICSCEQVK
YYEYNNAEKKNNKSLCHEKGNDRTWSKKYIKKLENGRTLEGVYVPPRRQQLCLYELFPIIIKNKNDITNAK
KELLETLQIVAEREAYYLWKQYHAHNDTTYLAHKKACCAIRGSFYDLEDIIKGNDLVHDEYTKYIDSKLNE
IFDSSNKNDIETKRARTDWWENEAIAVPNITGANKSDPKTIRQLVWDAMQSGVRKAIDEEKEKKKPNENFP
PCMGVQHIGIAKPQFIRWLEEWTNEFCEKYTKYFEDMKSNCNLRKGADDCDDNSNIECKKACANYTNWLNP
KRIEWNGMSNYYNKIYRKSNKESEDGKDYSMIMEPTVIDYLNKRCNGEINGNYICCSCKNIGENSTSGTVN
KKLQKKETQCEDNKGPLDLMNKVLNKMDPKYSEHKMKCTEVYLEHVEEQLKEIDNAIKDYKLYPLDRCFDD
KSKMKVCDLIGDAIGCKHKTKLDELDEWNDVDMRDPYNKYKGVLIPPRRRQLCFSRIVRGPANLRNLKEFK
EEILKGAQSEGKFLGNYYNEDKDKEKALEAMKNSFYDYEYIIKGSDMLTNIQFKDIKRKLDRLLEKETNNT
EKVDDWWETNKKSIWNAMLCGYKKSGNKIIDPSWCTIPTTETPPQFLRWIKEWGTNVCIQKEEHKEYVKSK
CSNVTNLGAQESESKNCTSEIKKYQEWSRKRSIQWEAISEGYKKYKGMDEFKNTFKNIKEPDANEPNANEY
LKKHCSKCPCGFNDMQEITKYTNIGNEAFKQIKEQVDIPAELEDVIYRLKHHEYDKGNDYICNKYKNINVN
MKKNNDDTWTDLVKNSSDINKGVLLPPRRKNLFLKIDESDICKYKRDPKLFKDFIYSSAISEVERLKKVYG
EAKTKVVHAMKYSFADIGSIIKGDDMMENNSSDKIGKILGDGVGQNEKRKKWWDMNKYHIWESMLCGYKHA
YGNISENDRKMLDIPNNDDEHQFLRWFQEWTENFCTKRNELYENMVTACNSAKCNTSNGSVDKKECTEACK
NYSNFILIKKKEYQSLNSQYDMNYKETKAEKKESPEYFKDKCNGECSCLSEYFKDETRWKNPYETLDDTEV
KNNCMCKPPPPASNNTSDILQKTIPFGIALALGSIAFLFMKKKPKTPVDLLRVLDIPKGDYGIPTPKSSNR
YIPYASDRYKGKTYIYMEGDTSGDDDKYIWDL
>FCR3 complete 2734 aa extracellular part (577 aa highlighted
corr. ID1-DBL2b)
(SEQ ID NO: 56)
MDSTSTIANKIEEYLGAKSDDSKIDELLKADPSEVEYYRSGGDGDYLKNNICKITVNHSDSGKYDPCEKKL
PPYDDNDQWKCQQNSSDGSGKPENICVPPRRERLCTYNLENLKFDKIRDNNAFLADVLLTARNEGEKIVQN
HPDTNSSNVCNALERSFADLADIIRGTDQWKGTNSNLEKNLKQMFAKIRENDKVLQDKYPKDQKYTKLREA
WWNANRQKVWEVITCGARSNDLLIKRGWRTSGKSDRKKNFELCRKCGHYEKEVPTKLDYVPQFLRWLTEWI
EDFYREKQNLIDDMERHREECTREDHKSKEGTSYCSTCKDKCKKYCECVKKWKTEWENQENKYKDLYEQNK
NKTSQKNTSRYDDYVKDFFEKLEANYSSLENYIKGDPYFAEYATKLSFILNPSDANNPSGETANHNDEACN
CNESGISSVGQAQTSGPSSNKTCITHSSIKTNKKKECKDVKLGVRENDKDLKICVIEDTSLSGVDNCCCQD
LLGILQENCSDNKRGSSSNDSCDNKNQDECQKKLEKVFASLTNGYKCDKCKSGTSRSKKKWIWKKSSGNEE
GLQEEYANTIGLPPRTQSLYLGNLPKLENVCEDVKDINFDTKEKFLAGCLIVSFHEGKNLKKRYPQNKNSG
NKENLCKALEYSFADYGDLIKGTSIWDNEYTKDLELNLQNNFGKLFGKYIKKNNTAEQDTSYSSLDELRES
WWNTNKKYIWTAMKHGAEMNITTCNADGSVTGSGSSCDDIPTIDLIPQYLRFLQEWVENFCEQRQAKVKDV
ITNCKSCKESGNKCKTECKTKCKDECEKYKKFIEACGTAGGGIGTAGSPWSKRWDQIYKRYSKHIEDAKRN
RKAGTKNCGTSSTTNAAASTDENKCVQSDIDSFFKHLIDIGLTTPSSYLSNVLDDNICGADKAPWTTYTTY
TTTEKCNKERDKSKSQSSDTLVVVNVPSPLGNTPYRYKYACQCKIPTNEETCDDRKEYMNQWSCGSARTMK
RGYKNDNYELCKYNGVDVKPTTVRSNSSKLDGNDVTFFNLFEQWNKEIQYQIEQYMTNANISCIDEKEVLD
SVSDEGTPKVRGGYEDGRNNNTDQGTNCKEKCKCYKLWIEKINDQWGKQKDNYNKFRSKQIYDANKGSQNK
KVVSLSNFLFFSCWEEYIQKYFNGDWSKIKNIGSDTFEFLIKKCGNNSAHGEEIFNEKLKNAEKKCKENES
TDTNINKSETSCDLNATNYIRGCQSKTYDGKIFPGKGGEKQWICKDTIIHGDTNGACIPPRTQNLCVGELW
DKSYGGRSNIKNDTKELLKEKIKNAIHKETELLYEYHDTGTAIISKNDKKGQKGKNDPNGLPKGFCHAVQR
SFIDYKNMILGTSVNIYEHIGKLQEDIKKIIEKGTPQQKDKIGGVGSSTENVNAWWKGIEREMWDAVRCAI
TKINKKNNNSIFNGDECGVSPPTGNDEDQSVSWFKEWGEQFCIERLRYEQNIREACTINGKNEKKCINSKS
GQGDKIQGACKRKCEKYKKYISEKKQEWDKQKTKYENKYVGKSASDLLKENYPECISANFDFIFNDNIEYK
TYYPYGDYSSICSCEQVKYYKYNNAEKKNNKSLCYEKDNDMTWSKKYIKKLENGRSLEGVYVPPRRQQLCL
YELFPIIIKNEEGMEKAKEELLETLQIVAEREAYYLWKQYNPTGKGIDDANKKACCAIRGSFYDLEDIIKG
NDLVHDEYTKYIDSKLNEIFGSSDTNDIDTKRARTDWWENETITNGTDRKTIRQLVWDAMQSGVRYAVEEK
NENFPLCMGVEHIGIAKPQFIRWLEEWTNEFCEKYTKYFEDMKSKCDPPKRADTCGDNSNIECKKACANYT
NWLNPKRIEWNGMSNYYNKIYRKSNKESEGGKDYSMIMAPTVIDYLNKRCHGEINGNYICCSCKNIGAYNT
TSGTVNKKLQKKETECEEEKGPLDLMNEVLNKMDKKYSAHKMKCTEVYLEHVEEQLNEIDNAIKDYKLYPL
DRCFDDQTKMKVCDLIADAIGCKDKTKLDELDEWNDMDLRGTYNKHKGVLIPPRRRQLCFSRIVRGPANLR
SLNEFKEEILKGAQSEGKFLGNYYKEHKDKEKALEAMKNSFYDYEDIIKGTDMLTNIEFKDIKIKLDRLLE
KETNNTKKAEDWWKTNKKSIWNAMLCGYKKSGNKIIDPSWCTIPTTETPPQFLRWIKEWGTNVCIQKQEHK
EYVKSKCSNVTNLGAQASESNNCTSEIKKYQEWSRKRSIRWETISKRYKKYKRMDILKDVKEPDANTYLRE
HCSKCPCGFNDMEEMNNNEDNEKEAFKQIKEQVKIPAELEDVIYRIKHHEYDKGNDYICNKYKNIHDRMKK
NNGNFVTDNFVKKSWEISNGVLIPPRRKNLFLYIDPSKICEYKKDPKLFKDFIYWSAFTEVERLKKAYGGA
RAKVVHAMKYSFTDIGSIIKGDDMMEKNSSDKIGKILGDTDGQNEKRKKWWDMNKYHIWESMLCGYREAEG
DTETNENCRFPDIESVPQFLRWFQEWSENFCDRRQKLYDKLNSECISAECTNGSVDNSKCTHACVNYKNYI
LTKKTEYEIQTNKYDNEFKNKNSNDKDAPDYLKEKCNDNKCECLNKHIDDKNKTWKNPYETLEDTFKSKCD
CPKPLPSPIKPDDLPPQADEPFDPTILQTTIPFGIALALGSIAFLFMKVIYIYIYVCCICMYVCMYVCMYV
CMYVCMYVCMHVCMLCVYVIYVFKICIYIEKEKRKK
>SpyCatcher
(SEQ ID NO: 57)
GAMVDTLSGLSSEQGQSGDMTIEEDSATHIKFSKRDEDGKELAGATMELRDSSGKTISTWISDGQVKDFYL
YPGKYTFVETAAPDGYEVATAITFTVNEQGQVTVNGKATKGDAHI
>Spytag
(SEQ ID NO: 58)
AHIVMVDAYKPTK
>Minimal Spytag sequence
(SEQ ID NO: 59)
AHIVMVDA
> The β-strand of CnaB2 (K-Tag
(SEQ ID NO: 60)
ATHIKFSKRD
SpyLigase
(SEQ ID NO: 61)
HHHHHHDYDGQSGDGKELAGATMELRDSSGKTISTWISDGQVKDFYLYPGKYTFVETAAPDGYEVATAITF
TVNEQGQVTVNGKATKGGSGGSGGSGEDSATHI
>isopeptide Spy0128
(SEQ ID NO: 62)
TDKDMTITFTNKKDAE
>Split-Spy0128
(SEQ ID NO: 63)
ATTVHGETVVNGAKLTVTKNLDLVNSNALIPNTDFTFKIEPDTTVNEDGNKFKGVALNTPMTKVTYTNSDK
GGSNTKTAEFDFSEVTFEKPGVYYYKVTEEKIDKVPGVSYDTTSYTVQVHVLWNEEQQKPVATYIVGYKEG
SKVPIQFKNSLDSTTLTVKKKVSGTGGDRSKDFNFGLTLKANQYYKASEKVMIEKTTKGGQAPVQTEASID
QLYHFTLKDGESIKVTNLPVGVDYVVTEDDYKSEKYTTNVEVSPQDGAVKNIAGNSTEQETSTDKDMTI
>SpyCatcher-ΔN
(SEQ ID NO: 64)
EDSATHIKFSKRDEDGKELAGATMELRDSSGKTISTWISDGQVKDFYLYPGKYTFVETAAPDGYEVATAIT
FTVNEQGQVTVNGKATKGDAHI
>Inversed Spytag sequence
(SEQ ID NO: 65)
KTPKYADVMVIHA
>SdyCatcher_DANG_Short
(SEQ ID NO: 66)
MGSSHHHHHHSSGLVPRGSHMASMTGGQQMGRGSSGLSGETGQSGNTTIEEDSTTHVKFSKRDANGKELAG
AMIELRNLSGQTIQSWISDGTVKVFYLMPGTYQFVETAAPEGYELAAPITFTIDEKGQIWVDS

### EXAMPLE 1

### VAR2CSA Binds Trophobalst Cells in a CSA-Dependent Manner

Human trophoblast cells (HTR8) and human placental cells (BeWo) were grown to 70%-80% confluency in appropriate growth media and harvested in an EDTA detachment solution (Cellstripper). Cells were incubated with protein (200-12.5 nM) in PBS containing 2% fetal bovine serum (FBS) for 30 min at 4 C and binding was analyzed in a FACSCalibur (BD Biosciences) after a secondary incubation with an anti-V5-FITC antibody (Table 1). As a specificity control, protein was co-incubated with 400 μg/ml of CSA, which out-competed the binding of rVAR2 to the cells. As a binding control, cells were incubated with 200 nM of a non-binding recombinant protein (DBL4). Similar results for BeWo binding of rVAR2.

TABLE 1
CSA-dependent binding of recombinant VAR2CSA (rVAR2)
to human HTR8 trophoblast cells

| SAMPLE | BINDING (MFI) |
|---|---|
| 200 nM rVAR2 | 10244.9 |
| 100 nM rVAR2 | 6248.9 |
| 50 nM rVAR2 | 1938.9 |
| 25 nM rVAR2 | 1938.9 |
| 12.5 nM rVAr2 | 233.9 |
| CSA + 200 nM rVAR2 | 321.9 |
| 200 nM DBL4 | 54.9 |
| No FITC | 0 |

### EXAMPLE 2

### VAR2CSA Binds Trophobalst Cells Mixed Into a Blood Sample

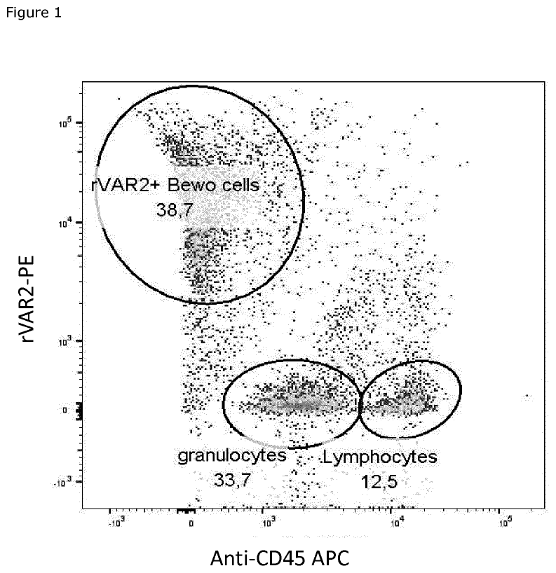

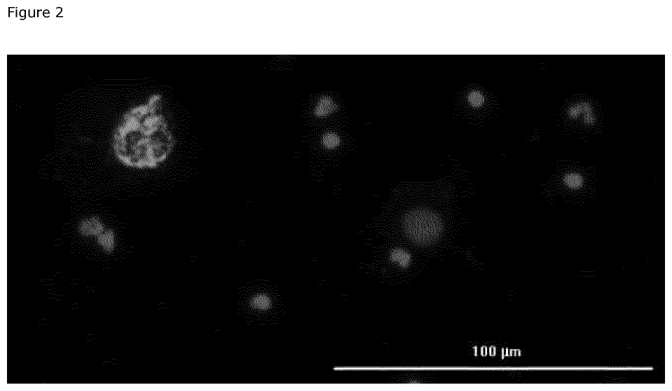

Blood samples were collected in EDTA Vacuette tubes and RBCs removed by lysis. BeWo were added directly to the blood or mixed with PBMCs after the RBC lysis. The cells were incubated with 100 nM PE-conjugated rVAR2 and anti-CD45-APC (cat. No. 17-0459-42, Invitrogen) for 30 minutes at 4° C. Following two wash steps in PBS with 2% FBS, cells were fixed in 4% PFA and data was acquired using a LSRII flow cytometer (BD). Mean fluorescence intensities were analyzed using FlowJo™ software. Flow cytometry analysis showed that rVAR2 bound specifically to BeWo cells with limited binding to other blood cells, proving the feasibility of detecting rare trophopblast cells in a blood sample (FIG. 1). As a specificity control, protein was co-incubated with 200 μg/ml of CSA, which out-competed the binding of rVAR2 to the cells. Furthermore, rVAR2 staining on Bewo cells in a background of PBMCs were analyzed by microscopy. This analysis showed that rVAR2 specifically recognizes trophoblast cells in a mixed blood cell population (FIG. 2).

### EXAMPLE 3

### VAR2CSA Binds Trophoblastic and Fetal Tissues from Early Pregnancy

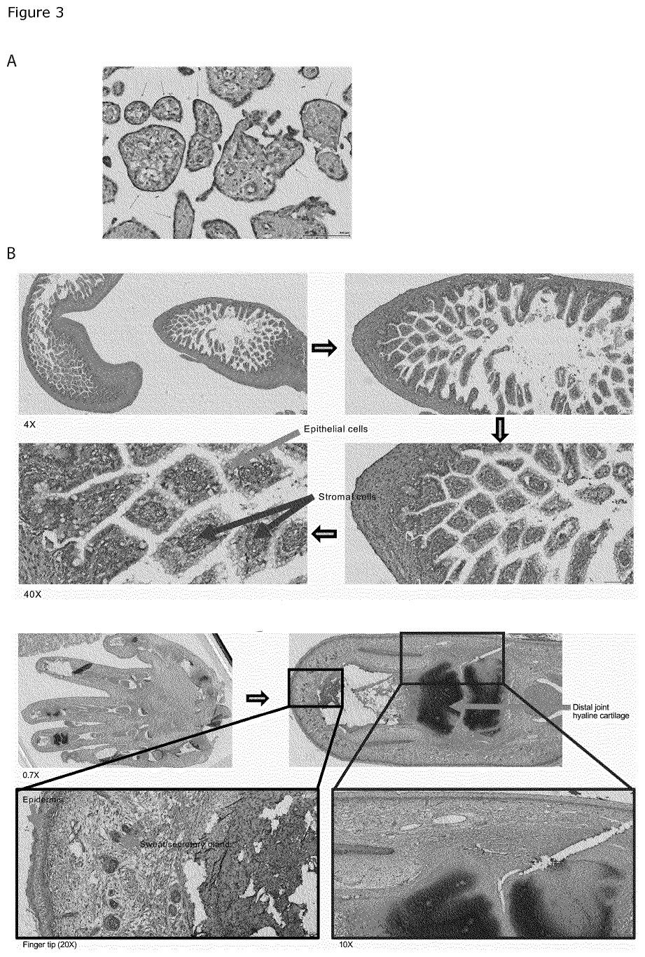

Using the Ventana Discovery platform, sectioned paraffin-embedded early pregnancy placental tissue samples were stained with 500 picomolar V5-tagged rVAR2 without antigen retrieval, followed by 1:700 monoclonal anti-V5 step and a anti-mouse-HRP detection step. FIG. 3 show VAR2CSA binding to the syncytiotrophoblast layer of placental tissue from early pregnancy as well as to fetal cells in a human embryo.

### EXAMPLE 4

### VAR2CSA Coated Beads Can Be Used to Isolate Trophoblast Cells in a Complex Blood Sample

The recombinantly expressed VAR2CSA (rVAR2) protein used in the rVAR2 CTC isolation method was designed to include a 13 amino acids peptide (SpyTag) from the fibronectin-binding protein Fba N-terminally or a 13 amino acid tag from fibronectin-binding protein in Streptococcus dysgalactiae (Sdy-tag), which enables covalent isopeptide bond formation to a biotinylated 12 kDa SpyCatcher or 12 kDa DANG catcher. The Catchers was produced in E. coli BI21 as a soluble poly-HIS tagged protein, and purified by Ni++ affinity chromatography. Purity was determined by SDS page and quality of protein was ensured by testing the capacity to form an isopeptide bond to a tagged protein. The tagged rVAR2 and biotinylated SpyCatcher or DANG catcher fragment were incubated at room temperature for 1 hour. After this step the protein was incubated with CELLection™ Biotin Binder Dynabeads® (4.5 μm) at room temperature for at least 30 min. resulting in rVAR2-coated beads (0.43 μg biotinylated protein/μl bead suspension). Remaining protein was removed by carefully washing the beads in PBS containing 0.1% BSA three times, each time using a neodymium magnet (10×12 mm) for dragging beads into a pellet. In a parallel experiment, magnetic beads were coated directly with rVAR2 through amine chemistry. There was no difference in the conjugation efficacy of the spycatcher or Dangcatcher versus directly coated beads and they were thus used interchangeable.

Prior to the spike-in experiments, primary human extravillious trophoblasts (EVTs) or human trophoblast cells were collected using enzyme-free CellStripper (Sigma-Aldrich) and re-suspended in culture medium. Cell concentration was measured by manually counting the number of viable cells in a 1:1 mixture with Trypan Blue solution (Sigma-Aldrich). The suspensions were subsequently spiked into blood to achieve the desired concentrations. Blood was received in EDTA-tubes and divided into aliquots of 5 mL. Red blood cells were lysed in 45 mL Red Blood Cell (RBC) lysis buffer containing 0.155M ammonium chloride, 0.01 M potassium hydrogen carbonate and 0.1 mM EDTA for 10 min. After centrifugation at 400×g for 8 min., the cell pellet was gently washed in PBS once. The centrifugation step was repeated, and finally cells were resuspended in DPBS with 0.5% BSA and 2 mM EDTA and transferred to a low retention microcentrifuge tube (Fisherbrand). Under these conditions, cells were incubated with ˜1.6E6 rVAR2-magnetic beads at 4° C. Trophoblast cells adhering to beads were retrieved by running the isolation protocol on the IsoFlux™ machine (Fluxion). Isolated trophoblast cells were hereafter retrieved in DPBS with 0.5% BSA and 2 mM EDTA and transferred to a low retention microcentrifuge tube (Fisherbrand). A neodymium cylinder magnet was used to drag cells bound to beads towards the bottom of the tube, enabling removal of supernatant. Cells were then fixed in 4% PFA for 5 minutes and added onto glass slides, on which a circle with the same size as the magnet had been drawn using a water repellent pen. When adding or removing buffer from cells the glass slide was placed on top of the magnet. Cells were blocked for 10 minutes in 10% normal donkey serum (NDS) prior to stain with PE-conjugated anti-CD45 [5B-1] antibody (Cat. No. 130-080-201, MACS Miltenyi Biotec) and PE-conjugated anti-CD66b antibody (cat. No 130-104-414, MACS Miltenyi Biotec). Hereafter cells were permeabilized using 0.2% Triton X-100 diluted in PBS containing 0.5% BSA and 2 mM EDTA. This step was followed by staining of the cells with FITC-conjugated anti-Cytokeratin [CK3-6H5] antibody (Cat. No. 130-080-101, MACS Miltenyi Biotec) or FITC-conjugated anti-PAPP-A (Cat. No. 006-01-02, ThermoFisher). To enable visualization of cell nuclei, cells were incubated in DAPI. The sample was mounted using Dako Faramount Aqueous Mounting Medium. The results show that rVAR2 magnetic beads can capture rare trophoblast cells mixed into a blood sample.

### EXAMPLE 5

### VAR2CSA Isolated Trophoblasts Can Be Picked for Single Cell Analyses

Sample preparation of rare trophoblast cells from a male origin and PBMC from a female origin (enriched for rVAR2 positive cells using rVAR2 beads) are scanned on the CellCelector. Single cells are isolated using a semi-automated micromanipulator, CellCelector (ALS GmbH, Jena, Germany). This system consists of an inverted fluorescent microscope (CKX41, Olympus, Tokyo, Japan) with a CCD camera system (XM10-IR, Olympus, Tokyo, Japan) and a vertical glass capillary of 30 μm in diameter on a robotic arm. ALS CellCelector Software 3.0, (ALS, Jena, Germany) is used for analysis. Labeled cell solutions are transferred to a glass slide and cells are allowed to settle. Then CK+ or PAPP-A+ cells are detected in the FITC channel at a 40× magnification. CK+ or PAPP-A+ cells are selected by the software and additionally recorded in the remaining channels (brightfield (BF), DAPI, and TRITC) at 40× magnification to verify morphology and CD45 negativity of isolated cells. Selected cells are aspirated with a 30 μm glass capillary and transferred into PCR tubes containing 100 μl of lysis buffer of the Guanidine Thiocyanate (GTC) Method. Total RNA is isolated by the GTC method using standard protocols. The purified RNA is used for cDNA synthesis using the SuperScript VILO Master Mix according to the manufacturer's recommendations (Cat. No. 1455280, Invitrogen), followed by preamplificaiton using AmpliTaq Gold 360 Master Mix (Cat. No. 4398881, Applied Biosystems). The picked cells are validated by this PCR method to have genes originating from a Y-chromosome, and will thus demonstrate that we can identify single rare trophoblast cells for genetic analyses. Similar data are generated on a CytoTrack device (see example 6) that scans a whole blood sample (without enrichment) distributed on a large disc. Rare cells are identified by hot spot staining, and in this case we stain with rVAR2 to identify placental cells among the PMBCs. A cytopicker on the CytoTrack allows for picking single cells for DNA analyses.

### EXAMPLE 6

### VAR2CSA Detects Trophoblast Cells in Blood

1000 trophoblast cells are mixed with 500,000 PBMC. Cells are incubated with 250 nM rVAR2 for 30 minutes at 4° C. and secondarily with anti-penta His Alexa Fluor 488 (Cat. No. 35310, Qiagen) and anti-human CD45 Cy5 (Cat. No. 19-0459, eBioscience). After fixation in 4% formaldehyde, cells are stained with DAPI (Cat. No. D1306, Life Technologies) and mounted on glass slides using FluorSave Reagent (Merck Millipore). rVAR2 positive cells are located using a CytoTrack CT4 Scanner. The resulting table of hotspots is subsequently analysed for morphology and rVAR2, DAPI and CD45 staining to validate PBMC and placental cell origin. Similar such a sample preparation is applied to the CellCelector for scanning and demonstrate that rVAR2 can identify rare placental cells without prior selection.

### EXAMPLE 7

### VAR2CSA Isolates Trophoblasts from Maternal Blood

Blood from a pregnant donor is sampled in K2 EDTA tubes and processed as described in example 4. Using the CellCelector as described in Example 5 we can isolate placental cells from these blood samples validated by PCR analyses to detect a Y-chromosome. Similarly we can identify them using a CytoTrack device and pick them for analyses using the cytopicker attached to the CytoTrack.

### EXAMPLE 8

### VAR2CSA Isolates Trophoblasts from Maternal Blood

Trophoblast enrichment was done on 9 ml blood using rVAR2-coating beads as follow:
spinning blood 1200 g/15 min in sepmate tube to remove red blood cells.
VAR2 (MP3425 corresponding to SEQ ID NO:1 with an N-terminal spytag sequence AHIVMVDAYKPTK of SEQ ID NO:58) were biotinylated with biotin-Spycatcher (MP 3168) and incubated with CELLection™ Dynabeads®, a magnetic bead coated with recombinant streptavidin (Thermo fisher Scientific #11533D).
rVAR2-coating of beads and cells were incubated for 30 min©4° C. with 360 degrees rotation.
complex of cells and beads were washed using magnet
DNA was extracted by Arcturus picoPure DNA extraction kit.
Nested PCR was performed using 3 sets of primers for 3 different regions on Y chromosome, DAZ1 gene, SRY gene, DYS14.

Primer sequences used:

SRY-Ext F
TACAGGCCATGCACAGAGAG
SRY-Ext R
TCTTGAGTGTGTGGCTTTCG
SRY-Int F
AGTATCGACCTCGTCGGAAG
SRY-Int R
TCTTGAGTGTGTGGCTTTCG
DYS14-Ext F
AGCCCTGATCACTGACGAAG
DYS14-Ext R
TGCAGAGATGAACAGGATGC
DYS14-Int F
AGGAAGACTGGGGCTAGAGG
DYS14-Int R
ACCTGTCAGGACAAGGTGGA
DAZ-Ext F
TACCTCCAAAGCACCAGAGC
DAZ-Ext R
AATCTACCCATTCCCGAACC
DAZ-Int F
TACCTCCAAAGCACCAGAGC
DAZ-Int R
TGAGGAGGCATCTGGAAATC

1. A method for the identification of a trophoblast and/or fetal cell in a biological sample, the method comprising:
a) contacting a biological sample comprising trophoblast and/or fetal cells expressing CSA with a VAR2CSA polypeptide, or a conjugate or fusion protein thereof;
b) detecting said VAR2CSA polypeptide or conjugate or fusion protein thereof specifically bound to said trophoblast and/or fetal cells expressing CSA.

2. The method according to claim 1, further comprising a step c) of isolating from the biological sample said trophoblast and/or fetal cells expressing CSA specifically bound to said VAR2CSA polypeptide or conjugate or fusion protein thereof.

3. The method according to any one of claim 1 or 2, further comprising a previous step of obtaining a biological sample comprising trophoblast and/or fetal cells expressing CSA from a subject, such as a pregnant female subject, such as a human female subject.

4. The method according to any one of claims 1-3, wherein said biological sample is or comprises peripheral blood.

5. The method according to any one of claims 1-4, wherein said biological sample is derived from a pregnant female subject, such as a human female subject.

6. The method according to any one of claims 1-5, which method detects a circulating trophoblast and/or fetal cell in the peripheral blood of a pregnant female, such as a human female subject.

7. The method according to any one of claims 1-6, wherein VAR2CSA polypeptide, or a conjugate or fusion protein thereof comprises a detectable label or diagnostic effector moiety, such as a fluorescent or radioactive label, and/or a carrier for detection, such as a magnetic bead.

8. The method according to any one of claims 1-7, wherein said VAR2CSA polypeptide consist of or comprises SEQ ID NO:55 or SEQ ID NO:56 or fragments or variants thereof with the ability to bind chondroitin sulfate A (CSA) that could be presented on a proteoglycans (CSPG).

9. The method according to any one of claims 1-8, wherein said VAR2CSA polypeptide is a fragment of VAR2CSA that consist of a sequential amino acid sequence of
a. ID1, and
b. DBL2Xb, and optionally
c. ID2a.

10. The method according to any one of claims 1-9, wherein said VAR2CSA polypeptide binds chondroitin sulfate A (CSA) on proteoglycans (CSPG) with an affinity as measured by a K_{D} lower than 100 nM, such as lower than 80 nM, such as lower than 70 nM, such as lower than 60 nM, such as lower than 50 nM, such as lower than 40 nM, such as lower than 30 nM, such as lower than 26 nM, such as lower than 24 nM, such as lower than 22 nM, such as lower than 20 nM, such as lower than 18 nM, such as lower than 16 nM, such as lower than 14 nM, such as lower than 12 nM, such as lower than 10 nM, such as lower than 9 nM, such as lower than 8 nM, such as lower than 7 nM, such as lower than 6 nM, or lower than 4 nM.

11. The method according to any one of claims 1-10, wherein said VAR2CSA polypeptide comprises an amino acid sequence having at least 70, 75, 80, 85, 90, or 95% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54.

12. The method according to any one of claims 1-11, wherein said VAR2CSA polypeptide comprises an amino acid sequence having at least 70, 75, 80, 85, 90, or 95% sequence identity with an amino acid sequence of 578-640 of SEQ ID NO:1, 593-656 of SEQ ID NO:3, 580-643 of SEQ ID NO:4, 577-640 of SEQ ID NO:5, 587-650 of SEQ ID NO:10, 580-643 of SEQ ID NO:11, 566-628 of SEQ ID NO:29, 585-647 of SEQ ID NO:34, 570-632 of SEQ ID NO:36, 576-639 of SEQ ID NO:37, 593-655 of SEQ ID NO:38, 604-667 of SEQ ID NO:41, 589-652 of SEQ ID NO:43, 566-628 of SEQ ID NO:44, 590-653 of SEQ ID NO:45, 574-637 of SEQ ID NO:48, 584-646 of SEQ ID NO:53, or 570-632 of SEQ ID NO:54.

13. The method according to any one of claims 1-12, wherein said VAR2CSA polypeptide comprises an amino acid sequence having at least 70, 75, 80, 85, 90, or 95% sequence identity with an amino acid sequence of SEQ ID NO:1, 2, 6, 8, 9, 12, 13, 14, 15, 16, 17, 18, 19, 20, 21, 22, 23, 24, 25, 26, 27, 28, 30, 31, 32, 33, 35, 39, 40, 42, 46, 47, 49, 50, 51, 52.

14. The method according to any one of claims 1-13, wherein said VAR2CSA polypeptide consists of an amino acid sequence having at least 70, 75, 80, 85, 90, or 95% sequence identity with any one amino acid sequence of 1-577 of SEQ ID NO:1, 1-592 of SEQ ID NO:3, 1-579 of SEQ ID NO:4, 1-576 of SEQ ID NO:5, 1-586 of SEQ ID NO:10, 1-579 of SEQ ID NO:11, 1-565 of SEQ ID NO:29, 1-584 of SEQ ID NO:34, 1-569 of SEQ ID NO:36, 1-575 of SEQ ID NO:37, 1-592 of SEQ ID NO:38, 1-603 of SEQ ID NO:41, 1-588 of SEQ ID NO:43, 1-565 of SEQ ID NO:44, 1-589 of SEQ ID NO:45, 1-573 of SEQ ID NO:48, 1-583 of SEQ ID NO:53, 1-569 of SEQ ID NO:54.

15. The method according to any one of claims 1-14, wherein said VAR2CSA polypeptide consists of an amino acid sequence selected from the list consisting of SEQ ID NO:1, 3-5, 10, 11, 29, 34, 36-38, 41, 43-45, 48, 53, 54.

16. The method according to any one of claims 1-15, wherein said VAR2CSA polypeptide consists of an amino acid sequence having a length of less than 700 amino acids, such as less than 690 amino acids, such as less than 680 amino acids, such as less than 670 amino acids, such as less than 660 amino acids, such as less than 650 amino acids, such as less than 640 amino acids, such as less than 630 amino acids, such as less than 620 amino acids, such as less than 610 amino acids, such as less than 600 amino acids, such as less than 590 amino acids, such as less than 580 amino acids, such as less than 570 amino acids.

17. The method according to any one of claims 1-16, wherein said VAR2CSA polypeptide or a conjugate or fusion protein thereof comprises a peptide part of a split-protein binding system.

18. The method according to any one of claims 1-17, wherein said peptide part of a split-protein binding system is selected from K-Tag (SEQ ID NO:60), SpyCatcher (SEQ ID NO:57), SpyCatcher-ΔN (SEQ ID NO:64), SpyTag (SEQ ID NO:58), Minimal Spytag sequence (SEQ ID NO:59), split-Spy0128 (SEQ ID NO:63), isopeptide Spy0128 (SEQ ID NO:62), or the peptide part of the Sdy/DANG catcher system (SEQ ID NO:66), or any inverse sequence thereof, or a variant thereof with sequence identity of at least about 80%, such as at least about 82, 84, 86, 88, 90, 92, 94, 96, 98, or 99%.

19. The method according to any one of claims 1-18, wherein said VAR2CSA polypeptide or a conjugate or fusion protein thereof is a conjugate with a magnetic bead.

20. A method of testing for pregnancy in a female subject, the method comprising identifying in a biological sample of the subject a trophoblast and/or fetal cell according to the method of claims 1-19, wherein a presence of said trophoblast and/or fetal cell is indicative of the pregnancy in the subject.

21. A method of testing a female subject for a trophoblastic disease, such as extrauterine pregnancy and gestational trophoblastic disease, the method comprising
(a) identifying and isolating in a biological sample of a pregnant female a trophoblast and/or fetal cell according to the method of claims 2-19; and
(b) subjecting said trophoblast and/or fetal cell to an assay specific to said trophoblastic disease, thereby diagnosing the disease.

22. A method of prenatally diagnosing or examining a conceptus, comprising
(a) identifying and isolating in a biological sample of a pregnant female a trophoblast and/or fetal cell according to the method of claims 2-19; and
(b) subjecting said trophoblast and/or fetal cell to a conceptus diagnostic assay, thereby prenatally diagnosing the conceptus.

23. The method according to claim 21 or 22, further comprising culturing said trophoblast and/or fetal cell prior to step (b) under conditions suitable for proliferation of said cell.

24. A method of generating a trophoblast and/or fetal cell culture, comprising:
(a) isolating trophoblast and/or fetal cells according to the method of any one of claims 2-19, and
(b) culturing said trophoblast and/or fetal cells under conditions suitable for proliferation of said trophoblasts, thereby generating the trophoblast culture.

25. The method according to claim 22, wherein said conceptus diagnostic assay is effected by a chromosomal analysis, such as detection of a Y chromosome.

26. The method according to claim 22, 23 or 25, wherein said diagnosing the conceptus comprises identifying at least one chromosomal and/or DNA abnormality, and/or determining a paternity of the conceptus.

27. A diagnostic composition comprising a VAR2CSA polypeptide bound to at least one trophoblast cell.

28. The diagnostic composition of claim 27, wherein said VAR2CSA polypeptide comprises:
(a) an amino acid sequence having at least 80% sequence identity to ID1 (positions 1-152 of SEQ ID NO:1) or an amino acid sequence having at least 30 sequential amino acids of ID1 (positions 1-152 of SEQ ID NO:1); and
(b) an amino acid sequence having at least 80% sequence identity to DBL2Xb (positions 153-577 of SEQ ID NO:1) or an amino acid sequence having at least 30 sequential amino acids of I DBL2Xb (positions 153-577 of SEQ ID NO:1).

29. The diagnostic composition of claim 27-28, wherein said VAR2CSA polypeptide comprises:
(a) an amino acid sequence having at least 80% sequence identity to ID1 (positions 1-152 of SEQ ID NO:1) or an amino acid sequence having at least 30 sequential amino acids of ID1(positions 1-152 of SEQ ID NO:1); and
(b) an amino acid sequence having at least 80% sequence identity to DBL2Xb (positions 153-577 of SEQ ID NO:1) or an amino acid sequence having at least 30 sequential amino acids of I DBL2Xb (positions 153-577 of SEQ ID NO:1), and
(c) an amino acid sequence having at least 80% sequence identity to ID2a (positions 578-640 of SEQ ID NO:1) or an amino acid sequence having at least 30 sequential amino acids of ID2a(positions 578-640 of SEQ ID NO:1).

30. The diagnostic composition of any one of claims 27-29, wherein said VAR2CSA polypeptide further comprises a detectable label or diagnostic effector moiety, such as a bead, such as a magnetic bead.

31. A method of treating a female subject for a trophoblastic disease, such as extrauterine pregnancy and gestational trophoblastic disease, the method comprising
(a) identifying and isolating in a biological sample of a pregnant female a trophoblast cell according to the method of claims 2-19;
(b) subjecting said trophoblast cell to an assay specific to said trophoblastic disease, thereby diagnosing the disease; and
c) administering a treatment to said female subject, wherein said treatment is specific for said disease diagnosis.

32. A method of treating a female subject for a trophoblastic disease, such as extrauterine pregnancy and gestational trophoblastic disease, the method comprising:
(a) ordering a test for diagnosing a trophoblastic disase, wherein said test comprises
(i) identifying and isolating in a biological sample of a pregnant female a trophoblast cell according to the method of claims 2-19;
(ii) subjecting said trophoblast cell to an assay specific to said trophoblastic disease, thereby diagnosing the disease; and
(b) administering a treatment to said female subject, wherein said treatment is specific for said disease diagnosis.
